(19) United States
(12) Patent Application Publication
(10) Pub. No.: US 2012/0283297 A1
(43) Pub. Date: Nov. 8, 2012

(54) OXADIAZOLE SUBSTITUTED INDAZOLE DERIVATIVES FOR USE AS SPHINGOSINE 1-PHOSPHATE 1 (S1P1) RECEPTOR AGONISTS

(75) Inventors: James Bailey, Stevenage (GB); Nigel Paul King, Singapore (SG); Xichen Lin, Shanghai (CN); Feng Ren, Shanghai (CN); Kheng-Chuan Tan, Singapore (SG); Sing Yeung Mak, Singapore (SG)

(73) Assignee: GLAXO WELLCOME HOUSE, Greenford, Middlesex (GB)

(21) Appl. No.: 13/516,908

(22) PCT Filed: Dec. 16, 2010
(86) PCT No.: PCT/CN2010/002059

§ 371 Date: Jun. 18, 2012

(30) Foreign Application Priority Data
Dec. 18, 2009 (CN) PCT/CN2009/075720

(51) Int. Cl. A61K 31/4245; C07D 413/14; C07D 413/10; A61P 25/00; A61P 29/00; A61P 11/06; A61P 19/02; A61P 1/00; A61P 1/04; A61P 17/06; A61P 35/00; A61P 35/04; A61P 31/12; A61P 3/10; A61K 31/4439

(52) U.S. Cl. 514/338; 514/364; 546/269.4; 548/131

(57) ABSTRACT

Oxadiazole substituted indazole derivatives of formula (I) or pharmaceutical salts thereof having pharmacological activity, processes for their preparation, pharmaceutical compositions containing them and their uses in the treatment of various disorders mediated by S1P1 receptors are disclosed.

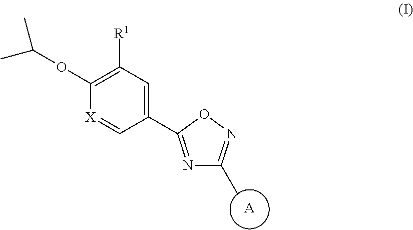

The present invention relates to novel compounds having pharmacological activity, processes for their preparation, pharmaceutical compositions containing them and their use in the treatment of various disorders.

Sphingosine 1-phosphate (S1P) is a bioactive lipid mediator formed by the phosphorylation of sphingosine by sphingosine kinases and is found in high levels in the blood. It is produced and secreted by a number of cell types, including those of hematopoietic origin such as platelets and mast cells (Okamoto et al 1998 J Biol Chem 273(42):27104; Sanchez and Hla 2004, J Cell Biochem 92:913). It has a wide range of biological actions, including regulation of cell proliferation, differentiation, motility, vascularisation, and activation of inflammatory cells and platelets (Pyne and Pyne 2000, Biochem J. 349: 385). Five subtypes of SIP responsive receptor have been described, S1P1 (Edg-1), S1P2 (Edg-5), S1P3 (Edg-3), S1P4 (Edg-6), and S1P5 (Edg-8), forming part of the G-protein coupled endothelial differentiation gene family of receptors (Chun et al 2002 Pharmacological Reviews 54:265, Sanchez and Hla 2004 J Cellular Biochemistry, 92:913). These 5 receptors show differential mRNA expression, with S1P1-3 being widely expressed, S1P4 expressed on lymphoid and hematopoietic tissues and S1P5 primarily in brain and to a lower degree in spleen. They signal via different subsets of G proteins to promote a variety of biological responses (Kluk and Hla 2002 Biochem et Biophysica Acta 1582:72, Sanchez and Hla 2004, J Cellular Biochem 92:913).

Proposed roles for the S1P1 receptor include lymphocyte trafficking, cytokine induction/suppression and effects on endothelial cells (Rosen and Goetzl 2005 Nat Rev Immunol. 5:560). Agonists of the S1P1 receptor have been used in a number of autoimmune and transplantation animal models, including Experimental Autoimmune Encephalomelitis (EAE) models of MS, to reduce the severity of the induced disease (Brinkman et al 2003 JBC 277:21453; Fujino et al 2003 J Pharmacol Exp Ther 305:70; Webb et al 2004 J Neuroimmunol 153:108; Rausch et al 2004 J Magn Reson Imaging 20:16). This activity is reported to be mediated by the effect of S1P1 agonists on lymphocyte circulation through the lymph system. Treatment with S1P1 agonists results in the sequestration of lymphocytes within secondary lymphoid organs such as the lymph nodes, inducing a reversible peripheral lymphopoenia in animal models (Chiba et al 1998, J Immunology 160:5037, Forrest et al 2004 J Pharmacol Exp Ther 309:758; Sanna et al 2004 JBC 279:13839). Published data on agonists suggests that compound treatment induces loss of the S1P1 receptor from the cell surface via internalisation (Graler and Goetzl 2004 FASEB J 18:551; Matloubian et al 2004 Nature 427:355; Jo et al 2005 Chem Biol 12:703) and it is this reduction of S1P1 receptor on immune cells which contributes to the reduction of movement of T cells from the lymph nodes back into the blood stream.

S1P1 gene deletion causes embryonic lethality. Experiments to examine the role of the S1P1 receptor in lymphocyte migration and trafficking have included the adoptive transfer of labelled S1P1 deficient T cells into irradiated wild type mice. These cells showed a reduced egress from secondary lymphoid organs (Matloubian et al 2004 Nature 427:355).

S1P1 has also been ascribed a role in endothelial cell junction modulation (Allende et al 2003 102:3665, Blood Singelton et al 2005 FASEB J 19:1646). With respect to this endothelial action, S1P1 agonists have been reported to have an effect on isolated lymph nodes which may be contributing to a role in modulating immune disorders. S1P1 agonists caused a closing of the endothelial stromal ‘gates’ of lymphatic sinuses which drain the lymph nodes and prevent lymphocyte egress (Wei wt al 2005, Nat. Immunology 6:1228).

The immunosuppressive compound FTY720 (JP11080026-A) has been shown to reduce circulating lymphocytes in animals and man, have disease modulating activity in animal models of immune disorders and reduce remission rates in relapsing remitting Multiple Sclerosis (Brinkman et al 2002 JBC 277:21453, Mandala et al 2002 Science 296:346, Fujino et al 2003 J Pharmacology and Experimental Therapeutics 305:45658, Brinkman et al 2004 American J Transplantation 4:1019, Webb et al 2004 J Neuroimmunology 153:108, Morris et al 2005 Eur J Immunol 35:3570, Chiba 2005 Pharmacology and Therapeutics 108:308, Kahan et al 2003, Transplantation 76:1079, Kappos et al 2006 New Eng J Medicine 335:1124). This compound is a prodrug that is phosphorylated in vivo by sphingosine kinases to give a molecule that has agonist activity at the S1P1, S1P3, S1P4 and S1P5 receptors. Clinical studies have demonstrated that treatment with FTY720 results in bradycardia in the first 24 hours of treatment (Kappos et al 2006 New Eng J Medicine 335:1124). The bradycardia is thought to be due to agonism at the S1P3 receptor, based on a number of cell based and animal experiments. These include the use of S1P3 knock-out animals which, unlike wild type mice, do not demonstrate bradycardia following FTY720 administration and the use of S1P1 selective compounds. (Hale et al 2004 Bioorganic & Medicinal Chemistry Letters 14:3501, Sanna et al 2004 JBC 279:13839, Koyrakh et al 2005 American J Transplantation 5:529)

Hence, there is a need for S1P1 receptor agonist compounds with selectivity over S1P3 which might be expected to show a reduced tendency to induce bradycardia.

The following patent applications describe oxadiazole derivatives as S1P1 agonists: WO03/105771, WO05/058848, WO06/047195, WO06/100633, WO06/115188, WO06/131336, WO07/024,922 and WO07/116,866.

The following patent application describes indole-oxadiazole derivatives as antipicornaviral agents: WO96/009822. The following patent applications describe indole-carboxylic acid derivatives as leukotriene receptor antagonists, pesticides and agrochemical fungicides respectively: WO06/090817, EP 0 439 785 and DE 39 39 238.

International patent applications WO08/074,821 and WO08/76356 describe oxadiazole-indole derivatives as S1P1 agonists.

A structurally novel class of compounds has now been found which provides agonists of the S1P1 receptor.

The present invention therefore provides compounds of formula (I) or a salt thereof:

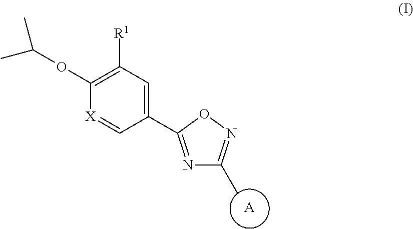

wherein

### X is CH or N;

R¹ is chloro or cyano;
A is a bicyclic ring selected from:

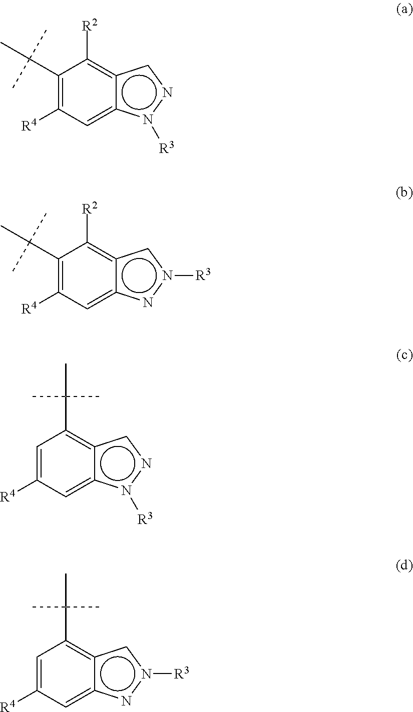

R² is hydrogen or methyl;
R³ is hydrogen, (CH₂)₂₋₄COOH, CH₂CH(CH₃)COOH, CH₂CH(OH)COOH or C(CH₂OCH₂)CH₂COOH; and
R⁴ is hydrogen, methyl, ethyl, fluoro, chloro or methoxy.

In one embodiment X is CH. In another embodiment X is N.

In one embodiment R¹ is chloro. In another embodiment R¹ is CN.

In one embodiment A is (a). In another embodiment A is (b). In another embodiment A is (c). In another embodiment A is (d).

In one embodiment R² is hydrogen. In another embodiment R² is methyl.

In one embodiment R³ is hydrogen. In another embodiment R³ is (CH₂)₂₋₄COOH. In another embodiment R³ is CH₂CH(CH₃)COOH. In another embodiment R³ is CH₂CH(OH)COOH. In another embodiment R³ is C(CH₂OCH₂)CH₂COOH.

In one embodiment R⁴ is hydrogen. In another embodiment R⁴ is methyl. In another embodiment R⁴ is ethyl. In another embodiment R⁴ is fluoro. In another embodiment R⁴ is chloro. In another embodiment R⁴ is methoxy.

In one embodiment

### X is CH or N;

R¹ is chloro or cyano;

### A is (a) or (b);

R² is hydrogen or methyl;
R³ is hydrogen or (CH₂)₂₋₃COOH;

R⁴ is hydrogen, methyl, ethyl, fluoro, chloro or methoxy.

In one embodiment

### X is CH or N;

R¹ is chloro or cyano;

### A is (a);

R² is hydrogen or methyl;
R³ is hydrogen or (CH₂)₂₋₃COOH;
R⁴ is hydrogen, methyl, ethyl, flouro, chloro or methoxy.

In one embodiment

### X is CH or N;

R¹ is chloro or cyano;

### A is (b);

R² is hydrogen;
R³ is hydrogen or (CH₂)₃COOH;
R⁴ is hydrogen, methyl, ethyl, fluoro, chloro or methoxy.

In one embodiment

### X is CH or N;

R¹ is chloro or cyano;

### A is (c) or (d);

R² is hydrogen;
R³ is hydrogen (CH₂)₄COOH or CH₂CH(CH₃)COOH, CH₂CH(OH)COOH or C(CH₂OCH₂)CH₂COOH;
R⁴ is hydrogen.

In one embodiment

### X is CH or N;

R¹ is chloro or cyano;

### A is (c);

R² is hydrogen;
R³ is hydrogen, (CH₂)₄COOH or CH₂CH(CH₃)COOH, CH₂CH(OH)COOH or C(CH₂OCH₂)CH₂COOH;
R⁴ is hydrogen.

In one embodiment

### X is CH or N;

R¹ is chloro or cyano;

### A is (d).

R² is hydrogen;
R³ is hydrogen or (CH₂)₄COOH;
R⁴ is hydrogen.

In a further aspect, this invention provides processes for preparation of a compound of formula (I).

In certain of the compounds of formula (I), dependent upon the nature of the substituent there are chiral carbon atoms and therefore compounds of formula (I) may exist as stereoisomers. The invention extends to all optical isomers such as stereoisomeric forms of the compounds of formula (I) including enantiomers, diastereoisomers and mixtures thereof, such as racemates. The different stereoisomeric forms may be separated or resolved one from the other by conventional methods or any given isomer may be obtained by conventional stereoselective or asymmetric syntheses.

Certain of the compounds herein can exist in various tautomeric forms and it is to be understood that the invention encompasses all such tautomeric forms.

Suitable compounds of formula (I) are:
5-(5-{3-chloro-4-[(1-methylethyl)oxy]phenyl}-1,2,4-oxadiazol-3-yl)-1H-indazole
5-[3-(1H-indazol-5-yl)-1,2,4-oxadiazol-5-yl]-2-[(1-methylethyl)oxy]benzonitrile
5-[3-(1H-indazol-4-yl)-1,2,4-oxadiazol-5-yl]-2-[(1-methylethyl)oxy]benzonitrile
4-(5-{3-chloro-4-[(1-methylethyl)oxy]phenyl}-1,2,4-oxadiazol-3-yl)-1H-indazole
3-[4-(5-{3-chloro-4-[(1-methylethyl)oxy]phenyl}-1,2,4-oxadiazol-3-yl)-1H-indazol-1-yl]-2-methylpropanoic acid
5-[4-(5-{3-chloro-4-[(1-methylethyl)oxy]phenyl}-1,2,4-oxadiazol-3-yl)-1H-indazol-1-yl]pentanoic acid
5-[4-(5-{3-chloro-4-[(1-methylethyl)oxy]phenyl}-1,2,4-oxadiazol-3-yl)-2H-indazol-2-yl]pentanoic acid
{3-[4-(5-{3-chloro-4-[(1-methylethyl)oxy]phenyl}-1,2,4-oxadiazol-3-yl)-1H-indazol-1-yl]-3-oxetanyl}acetic acid
3-[4-(5-{3-chloro-4-[(1-methylethyl)oxy]phenyl}-1,2,4-oxadiazol-3-yl)-1H-indazol-1-yl]-2-hydroxypropanoic acid
3-[5-(5-{5-chloro-6-[(1-methylethyl)oxy]-3-pyridinyl}-1,2,4-oxadiazol-3-yl)-1H-indazol-1-yl]propanoic acid
5-(5-{5-chloro-6-[(1-methylethyl)oxy]-3-pyridinyl}-1,2,4-oxadiazol-3-yl)-1H-indazole
4-[5-(5-{5-chloro-6-[(1-methylethyl)oxy]-3-pyridinyl}-1,2,4-oxadiazol-3-yl)-6-methyl-1H-indazol-1-yl]butanoic acid
4-[5-(5-{5-chloro-6-[(1-methylethyl)oxy]-3-pyridinyl}-1,2,4-oxadiazol-3-yl)-4-methyl-1H-indazol-1-yl]butanoic acid
4-[5-(5-{5-cyano-6-[(1-methylethyl)oxy]-3-pyridinyl}-1,2,4-oxadiazol-3-yl)-1H-indazol-1-yl]butanoic acid
5-(5-{5-chloro-6-[(1-methylethyl)oxy]-3-pyridinyl}-1,2,4-oxadiazol-3-yl)-6-methyl-1H-indazole
5-(5-{5-chloro-6-[(1-methylethyl)oxy]-3-pyridinyl}-1,2,4-oxadiazol-3-yl)-6-fluoro-1H-indazole
4-[5-(5-{5-chloro-6-[(1-methylethyl)oxy]-3-pyridinyl}-1,2,4-oxadiazol-3-yl)-6-fluoro-1H-indazol-1-yl]butanoic acid
4-[5-(5-{5-chloro-6-[(1-methylethyl)oxy]-3-pyridinyl}-1,2,4-oxadiazol-3-yl)-6-fluoro-2H-indazol-2-yl]butanoic acid
6-chloro-5-(5-{5-chloro-6-[(1-methylethyl)oxy]-3-pyridinyl}-1,2,4-oxadiazol-3-yl)-1H-indazole
4-[6-chloro-5-(5-{5-chloro-6-[(1-methylethyl)oxy]-3-pyridinyl}-1,2,4-oxadiazol-3-yl)-1H-indazol-1-yl]butanoic acid
4-[6-chloro-5-(5-{5-chloro-6-[(1-methylethyl)oxy]-3-pyridinyl}-1,2,4-oxadiazol-3-yl)-2H-indazol-2-yl]butanoic acid
5-(5-{5-chloro-6-[(1-methylethyl)oxy]-3-pyridinyl}-1,2,4-oxadiazol-3-yl)-6-ethyl-1H-indazole
4-[5-(5-{5-chloro-6-[(1-methylethyl)oxy]-3-pyridinyl}-1,2,4-oxadiazol-3-yl)-6-ethyl-2H-indazol-2-yl]butanoic acid
4-[5-(5-{5-chloro-6-[(1-methylethyl)oxy]-3-pyridinyl}-1,2,4-oxadiazol-3-yl)-6-ethyl-1H-indazol-1-yl]butanoic acid
5-(5-{5-chloro-6-[(1-methylethyl)oxy]-3-pyridinyl}-1,2,4-oxadiazol-3-yl)-6-(methyloxy)-1H-indazole
3-[5-(5-{5-cyano-6-[(1-methylethyl)oxy]-3-pyridinyl}-1,2,4-oxadiazol-3-yl)-1H-indazol-1-yl]propanoic acid
4-[6-chloro-5-(5-{5-cyano-6-[(1-methylethyl)oxy]-3-pyridinyl}-1,2,4-oxadiazol-3-yl)-1H-indazol-1-yl]butanoic acid
4-[6-chloro-5-(5-{3-cyano-4-[(1-methylethyl)oxy]phenyl}-1,2,4-oxadiazol-3-yl)-1H-indazol-1-yl]butanoic acid
4-[5-(5-{3-cyano-4-[(1-methylethyl)oxy]phenyl}-1,2,4-oxadiazol-3-yl)-6-methyl-1H-indazol-1-yl]butanoic acid
4-[5-(5-{3-chloro-4-[(1-methylethyl)oxy]phenyl}-1,2,4-oxadiazol-3-yl)-6-methyl-1H-indazol-1-yl]butanoic acid
4-[5-(5-{3-cyano-4-[(1-methylethyl)oxy]phenyl}-1,2,4-oxadiazol-3-yl)-6-ethyl-1H-indazol-1-yl]butanoic acid
4-[5-(5-{3-cyano-4-[(1-methylethyl)oxy]phenyl}-1,2,4-oxadiazol-3-yl)-6-(methyloxy)-1H-indazol-1-yl]butanoic acid or salts thereof.

Pharmaceutically acceptable derivatives of compounds of formula (I) include any pharmaceutically acceptable salt, ester or salt of such ester of a compound of formula (I) which, upon administration to the recipient is capable of providing (directly or indirectly) a compound of formula (I) or an active metabolic or residue thereof.

The compounds of formula (I) can form salts. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. Certain of the compounds of formula (I) may form acid addition salts with one or more equivalents of the acid. The present invention includes within its scope all possible stoichiometric and non-stoichiometric forms. Salts may also be prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropylamine, tris(hydroxymethyl)aminomethane (TRIS, trometamol) and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

The compounds of formula (I) may be prepared in crystalline or non-crystalline form, and, if crystalline, may optionally be hydrated or solvated. This invention includes within its scope stoichiometric hydrates or solvates as well as compounds containing variable amounts of water and/or solvent.

Included within the scope of the invention are all salts, solvates, hydrates, complexes, polymorphs, prodrugs, radiolabelled derivatives, stereoisomers and optical isomers of the compounds of formula (I).

Pharmaceutically acceptable salts may be prepared conventionally by reaction with the appropriate acid or acid derivative.

The potencies and efficacies of the compounds of this invention for the S1P1 receptor can be determined by S1P1 Tango assay performed on the human cloned receptor as described herein. Compounds of formula (I) have demonstrated agonist activity at the S1P1 receptor, using functional assays described herein.

Compounds of formula (I) and their pharmaceutically acceptable salts are therefore of use in the treatment of conditions or disorders which are mediated via the S1P1 receptor. In particular the compounds of formula (I) and their pharmaceutically acceptable salts are of use in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

Compounds of formula (I) and their pharmaceutically acceptable salts are therefore of use in the treatment of multiple sclerosis.

Compounds of formula (I) and their pharmaceutically acceptable salts may also be of use in the treatment of Parkinson's Disease, Alzheimer's disease, Huntington's chorea, amyotrophic lateral sclerosis, spinal muscular atrophy, polyglutamine expansion disorders, vascular dementia, Down's syndrome, HIV dementia, dementia, ocular diseases including glaucoma, aged related macular degeneration, cataracts, traumatic eye injury, diabetic retinopathy, traumatic brain injury, stroke, tauopathies and hearing loss.

It is to be understood that “treatment” as used herein includes prophylaxis as well as alleviation of established symptoms.

Thus the invention also provides a compound of formula (I) or a pharmaceutically acceptable salt thereof, for use as a therapeutic substance, in particular in the treatment of the conditions or disorders mediated via the S1P1 receptor. In particular the invention provides a compound of formula (I) or a pharmaceutically acceptable salt thereof for use as a therapeutic substance in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemic-reperfusion injury, solid tumours, and tumour metastasis, diseases associated with angiogenesis, vascular diseases, pain conditions, acute viral diseases, inflammatory bowel conditions, insulin and non-insulin dependant diabetes. The invention further provides a method of treatment of conditions or disorders in mammals including humans which can be mediated via the S1P1 receptor, which comprises administering to the sufferer a therapeutically safe and effective amount of a compound of formula (I) or a pharmaceutically acceptable salt thereof.

Compounds of formula (I) and their pharmaceutically acceptable salts are of use as therapeutic substances in the treatment of multiple sclerosis.

In another aspect, the invention provides for the use of a compound of formula (I) or a pharmaceutically acceptable salt thereof in the manufacture of a medicament for use in the treatment of the conditions or disorders mediated via the S1P1 receptor

The invention provides a method of treatment of multiple sclerosis, which comprises administering to the sufferer a therapeutically safe and effective amount of a compound of formula (I) or a pharmaceutically acceptable salt thereof.

In order to use the compounds of formula (I) and pharmaceutically acceptable salts thereof in therapy, they will normally be formulated into a pharmaceutical composition in accordance with standard pharmaceutical practice. The present invention also provides a pharmaceutical composition, which comprises a compound of formula (I) or a pharmaceutically acceptable salt thereof, and a pharmaceutically acceptable carrier or excipient.

In a further aspect, the present invention provides a process for preparing a pharmaceutical composition, the process comprising mixing a compound of formula (I) or a pharmaceutically acceptable salt thereof and a pharmaceutically acceptable carrier or excipient.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents (e.g. pregelatinised maize starch, polyvinylpyrrolidone or hydroxypropyl methylcellulose); fillers (e.g. lactose, microcrystalline cellulose or calcium hydrogen phosphate); tabletting lubricants (e.g. magnesium stearate, talc or silica); disintegrants (e.g. potato starch or sodium starch glycollate); and acceptable wetting agents (e.g. sodium lauryl sulphate). The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents (e.g. sorbitol syrup, cellulose derivatives or hydrogenated edible fats), emulsifying agents (e.g. lecithin or acacia), non-aqueous vehicles (which may include edible oils e.g. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for oral administration may be suitably formulated to give controlled release of the active compound.

For parenteral administration, fluid unit dosage forms are prepared utilising a compound of the invention or pharmaceutically acceptable salts thereof and a sterile vehicle. Formulations for injection may be presented in unit dosage form e.g. in ampoules or in multi-dose, utilising a compound of the invention or pharmaceutically acceptable derivatives thereof and a sterile vehicle, optionally with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of formula (I) or pharmaceutically acceptable salts thereof may also be formulated in rectal compositions such as suppositories or retention enemas, e.g. containing conventional suppository bases such as cocoa butter or other glycerides.

The compounds of formula (I) or pharmaceutically acceptable salts thereof may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of formula (I) or pharmaceutically acceptable salts thereof, may be formulated as solutions for administration via a suitable metered or unitary dose device or alternatively as a powder mix with a suitable carrier for administration using a suitable delivery device. Thus compounds of formula (I) or pharmaceutically acceptable salts thereof may be formulated for oral, buccal, parenteral, topical (including ophthalmic and nasal), depot or rectal administration or in a form suitable for administration by inhalation or insufflation (either through the mouth or nose).

The compounds of formula (I) or pharmaceutically acceptable salts thereof may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

The composition may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, 1.0 to 500 mg or 1.0 to 200 mg and such unit doses may be administered more than once a day, for example two or three times a day.

Compounds of formula (I) or pharmaceutically acceptable salts thereof may be used in combination preparations. For example, the compounds of the invention may be used in combination with cyclosporin A, methotrexate, steroids, rapamycin, proinflammatory cytokine inhibitors, immunomodulators including biologicals or other therapeutically active compounds.

The subject invention also includes isotopically-labeled compounds, which are identical to those recited in formulas I and following, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹⁸F, ¹²³I and ¹²⁵I.

Compounds of the present invention and pharmaceutically acceptable salts of said compounds that contain the aforementioned isotopes and/or other isotopes of other atoms are within the scope of the present invention. Isotopically-labeled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula (I) and following of this invention can generally be prepared by carrying out the procedures disclosed in the Schemes and/or in the Examples below, by substituting a readily available isotopically labelled reagent for a non-isotopically labeled reagent.

In a further aspect, this invention provides processes for preparation of a compound of formula (I).

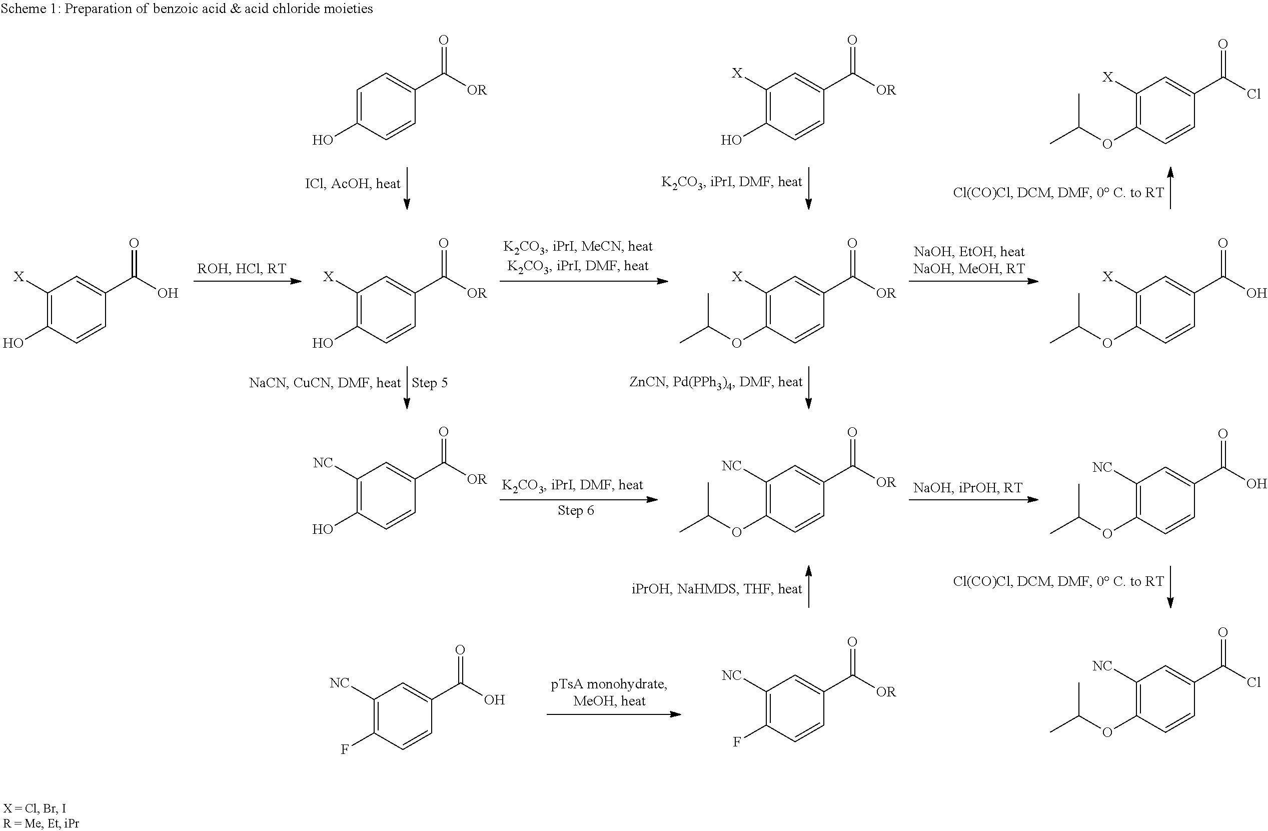

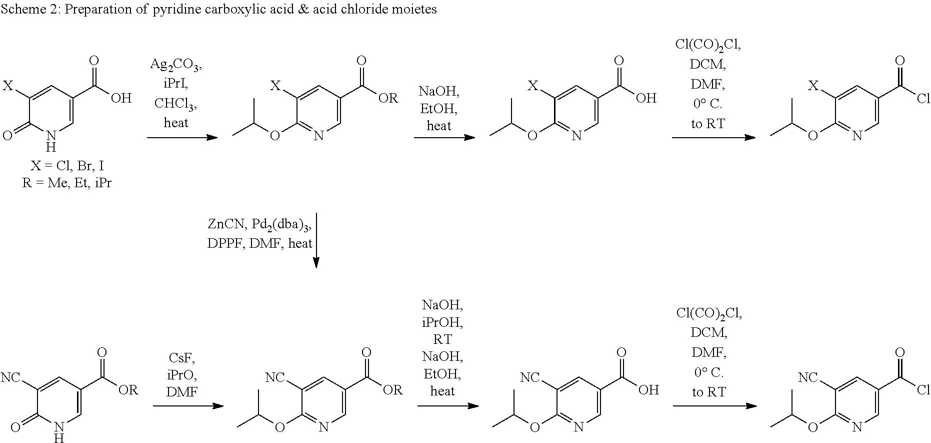

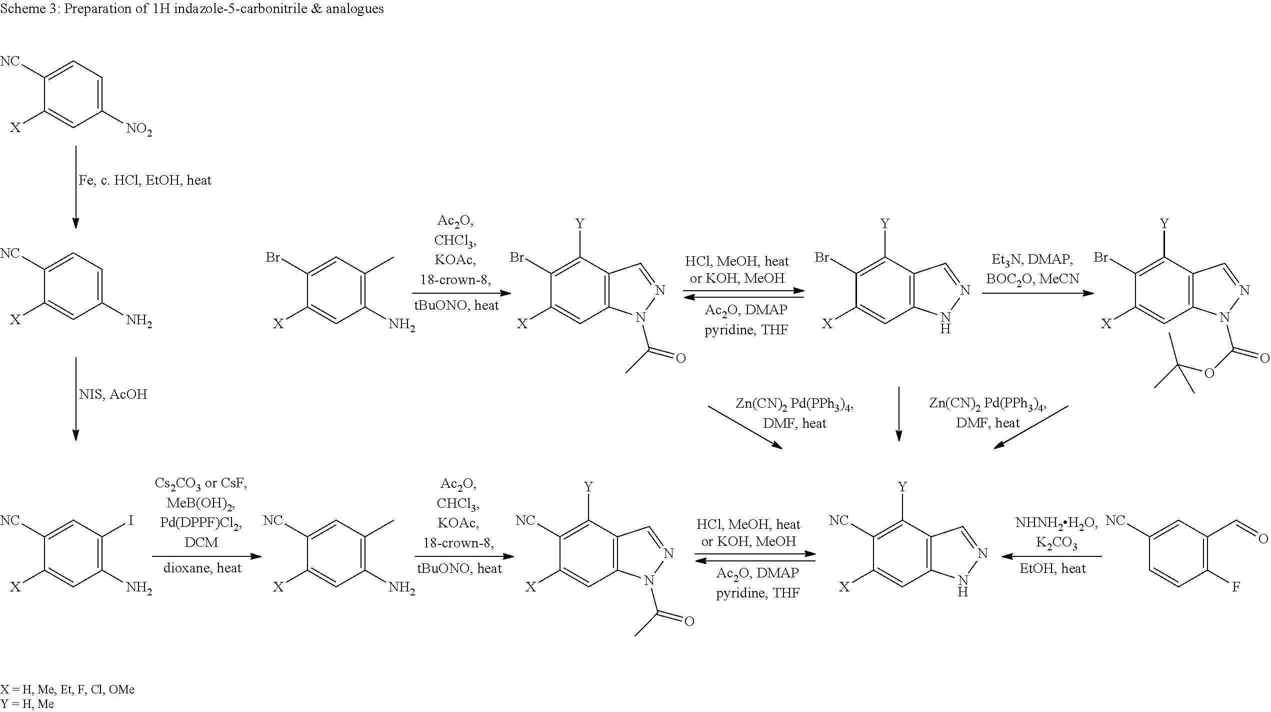

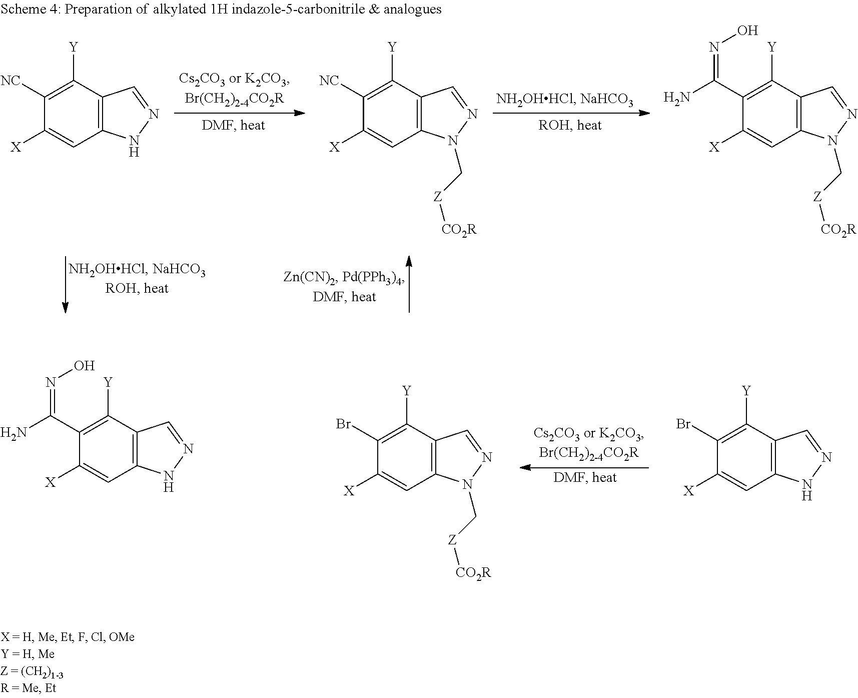

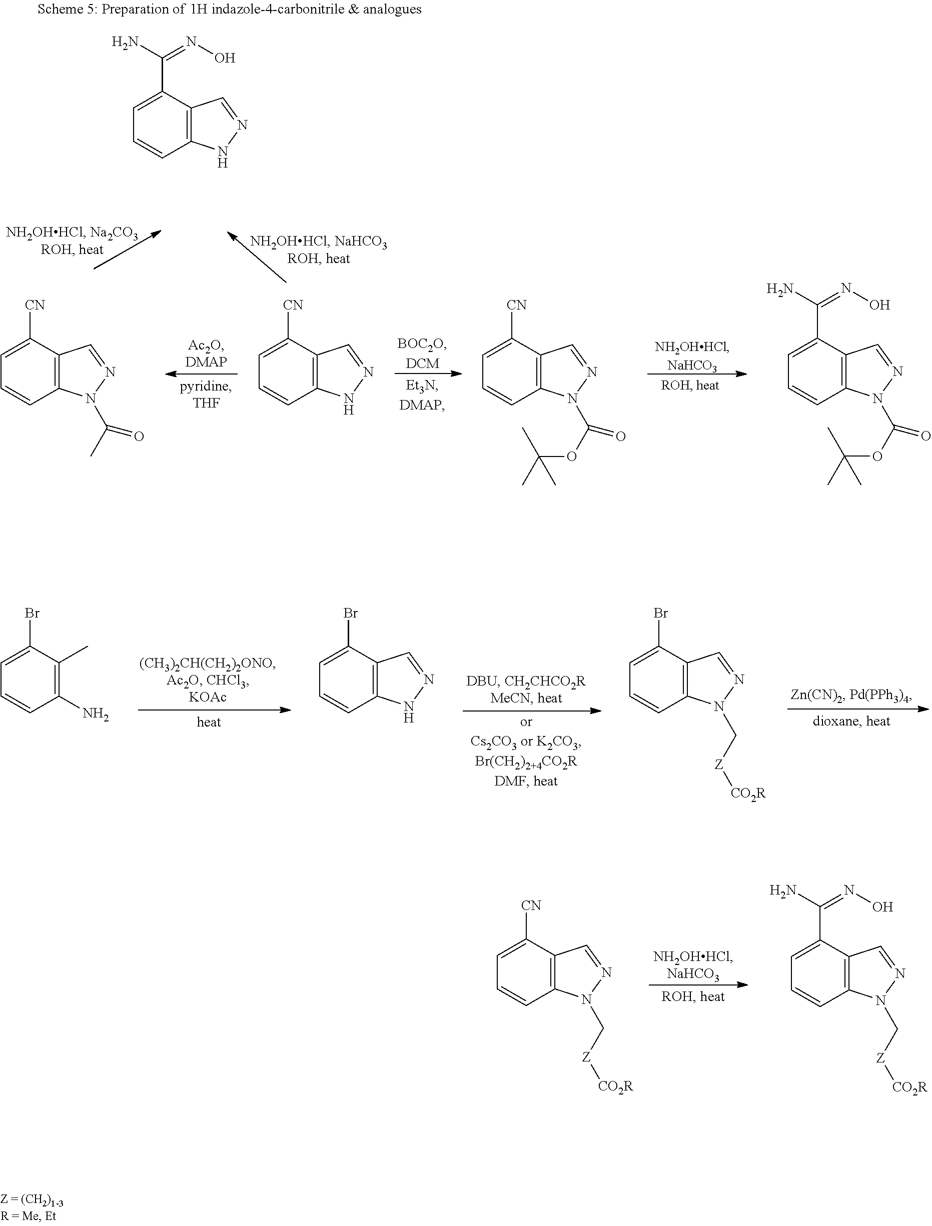

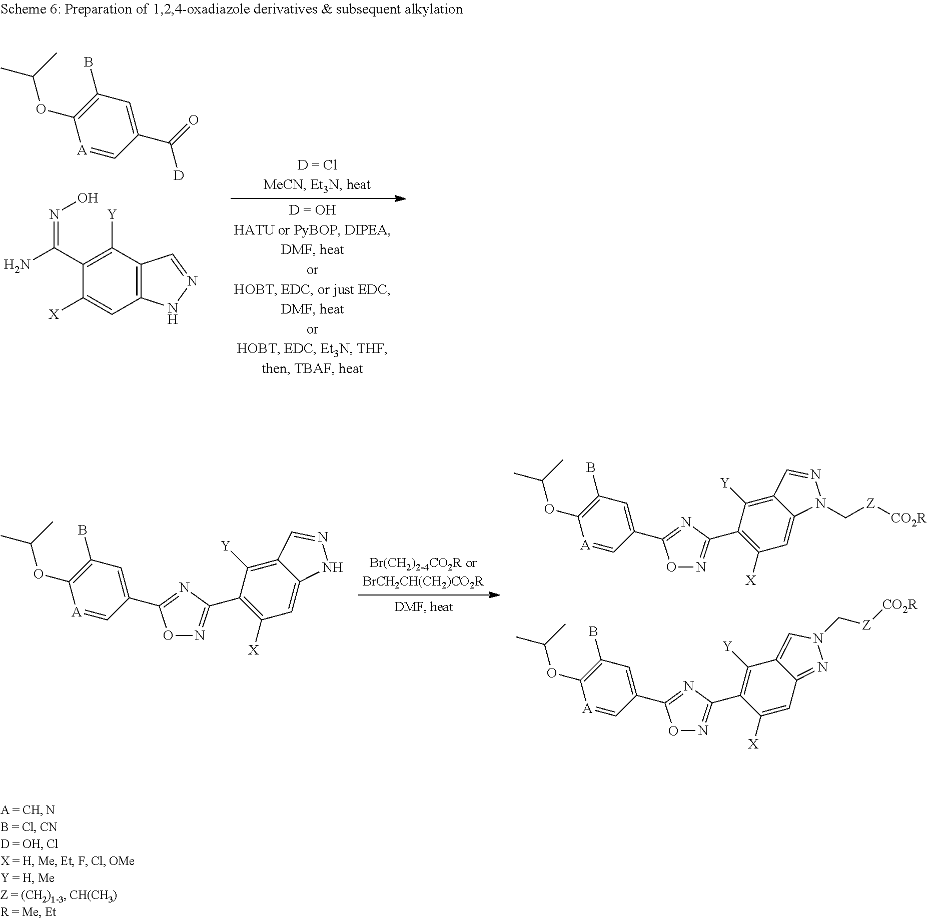

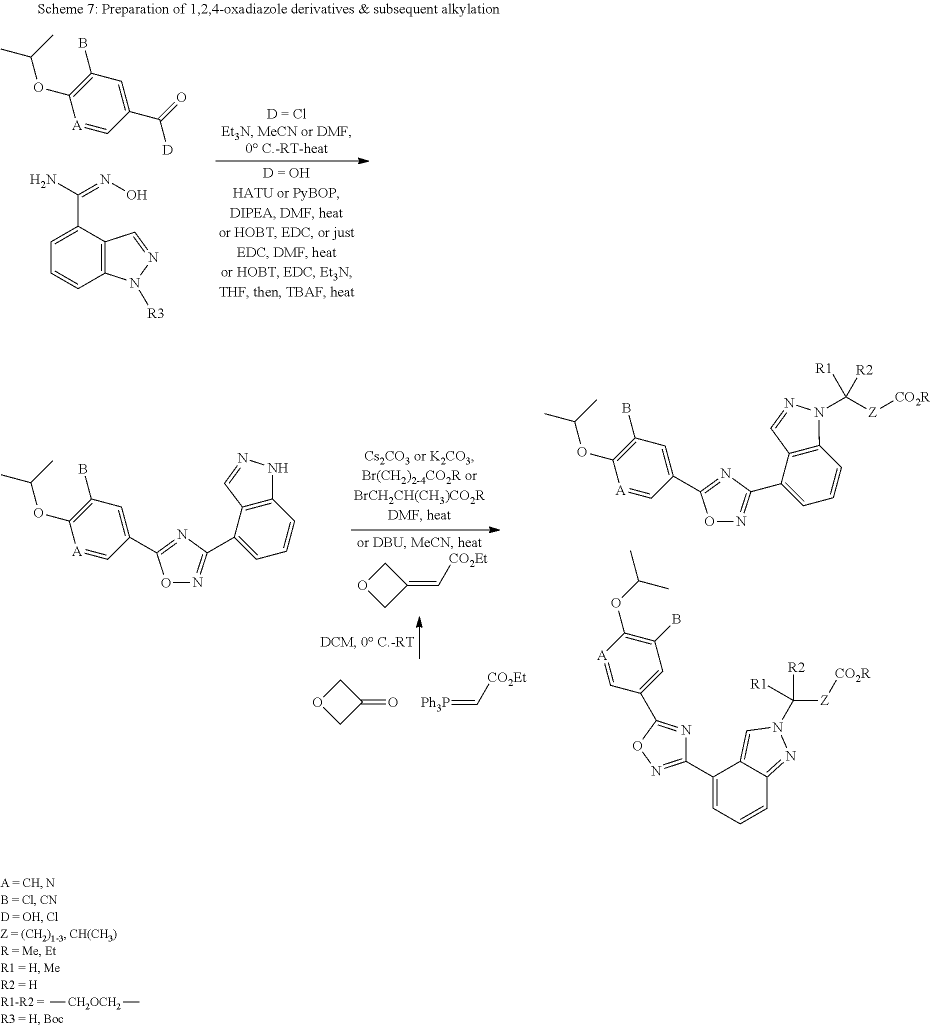

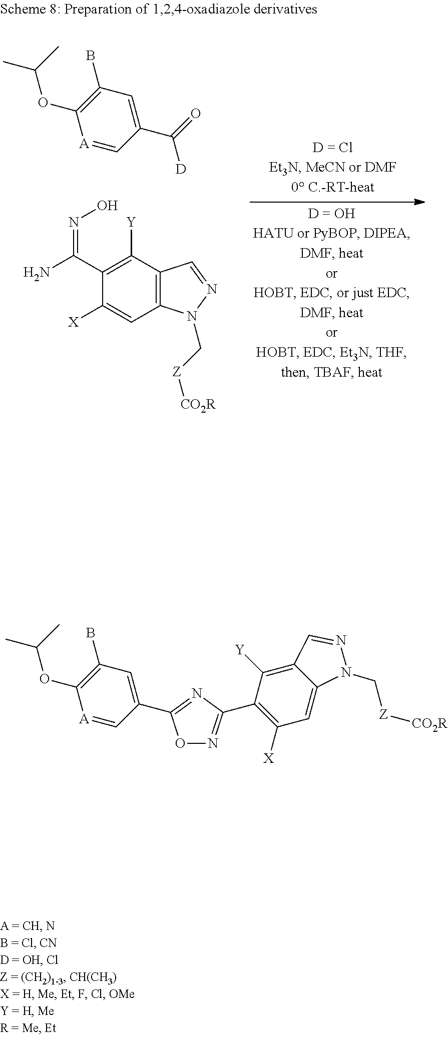

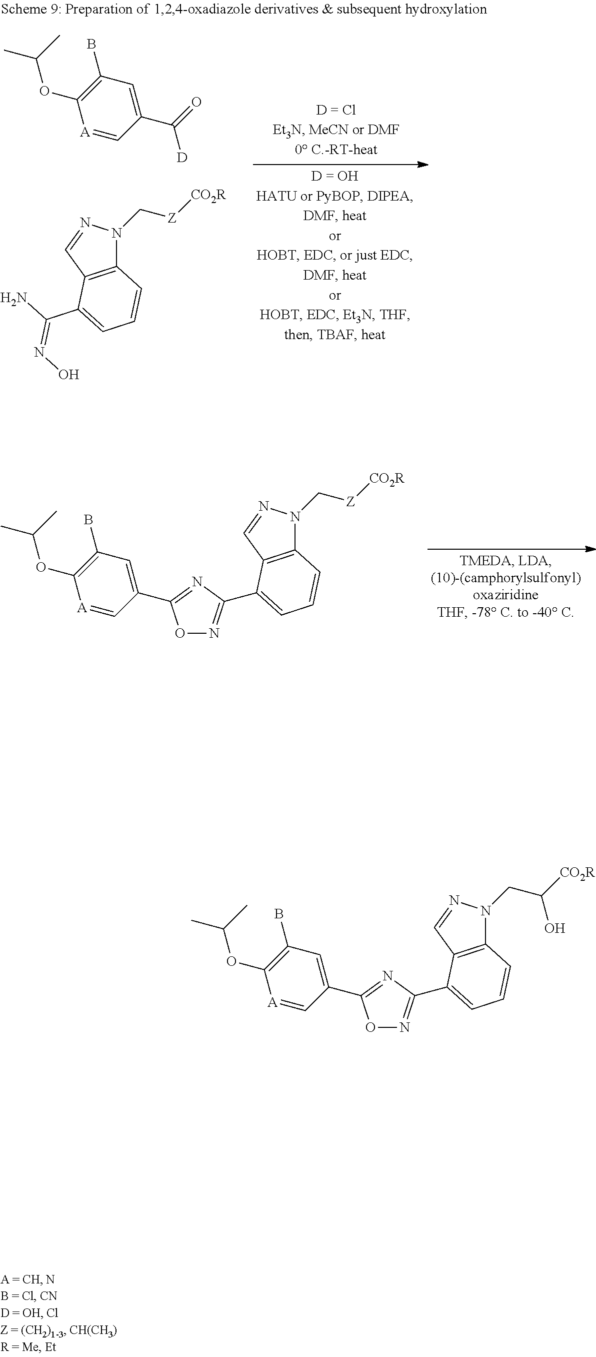

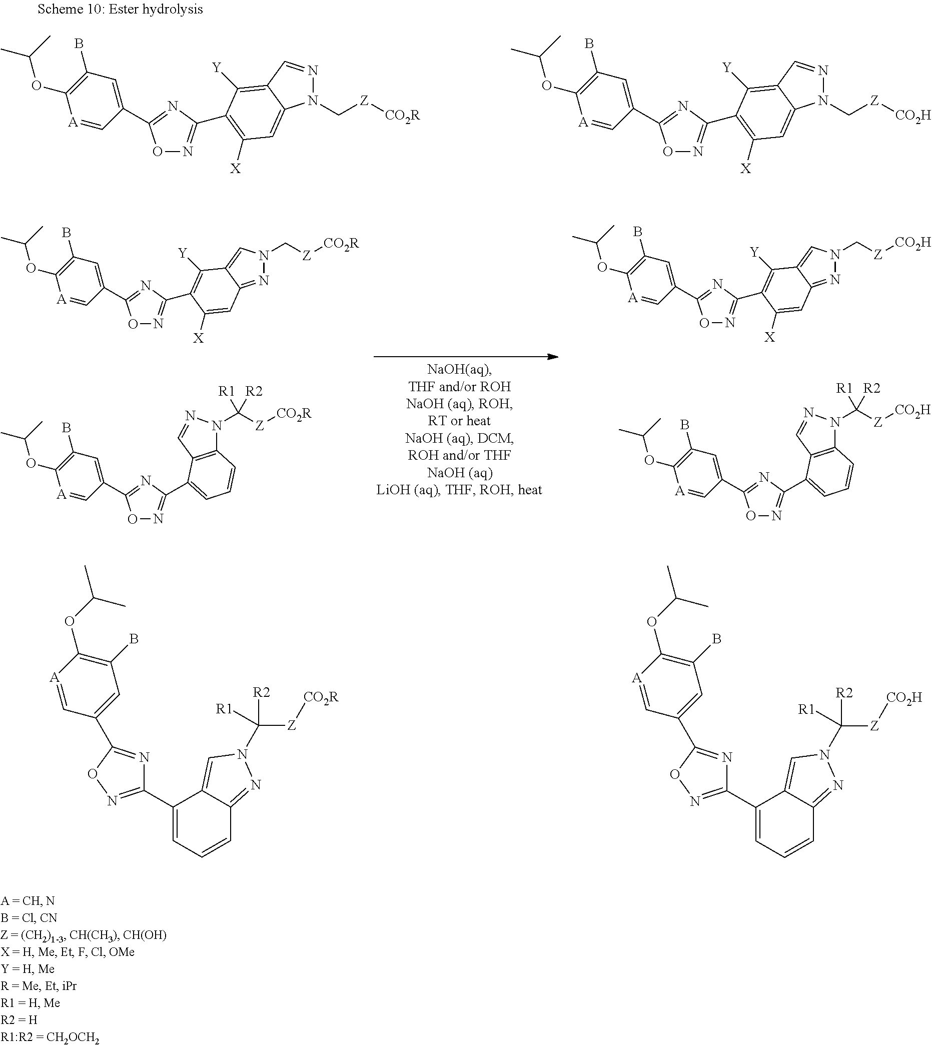

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of compounds of the invention.

### ABBREVIATIONS

DBU: 1,8-Diazabicyclo[5.4.0]undec-7-ene
DCM: Dichloromethane
DIPEA: N,N-diisopropylethylamine
DMF: N,N-Dimethylformamide
DPPF: 1,1-Bis(diphenylphosphino)ferrocene
EDC/EDCI: N-[3-(Dimethylamino)propyl]-N′-ethylcarbodiimide hydrochloride
h: Hour(s)
HATU o-(7-azabenzotriazol-1-yl)-N,N,N′,N′-tetramethyluronium hexafluorophosphate
HOBT: 1-Hydroxybenzotriazole
LDA: Lithium diisopropylamide
LCMS: Liquid Chromatography Mass Spectrometry
M: Molarity
MDAP: Mass Directed Auto Purification system
min: Minute(s)
PyBOP: (Benzotriazol-1-yloxy)tripyrrolidinophosphonium hexafluorophosphate
RT: Room temperature
Rt: Retention time
TBAF: Tetrabutylammonium fluoride
THF: Tetrahydrofuran
TMEDA: N,N,N′,N′-Tetramethylethylenediamine

### Conditions Used on the LCMS (A) and MDAP

### Basic Condition

Mobile phase: water 0.2% diethylamine-acetonitrile 0.2% diethylamine
Column: XBridge™ C18 30×100 mm-5 microns
Detection: MS and photodiode array detector (PDA)

### Acidic Condition

Mobile phase: water 0.2% formic acid-acetonitrile 0.2% formic acid
Column: XBridge™ C18 30×100 mm-5 microns
   Detection: MS and photodiode array detector (PDA)

### Conditions Used on the LCMS (B)

The HPLC analysis was conducted on an Acquity HPLC BEH C18 column (2.1 mm×50 mm i.d. 1.7 um packing diameter) at 40° C.

The solvents employed were:

A: 0.1% v/v solution of formic acid in water

B: 0.1% v/v solution of formic acid in acetonitrile

The gradient employed was:

| Time (min) | Flow rate (mL/min) | % A | % B |
|---|---|---|---|
| 0 | 1 | 97 | 3 |
| 1.5 | 1 | 0 | 100 |
| 1.9 | 1 | 0 | 100 |
| 2.0 | 1 | 97 | 3 |

The UV detection was an averaged signal from wavelength 210 nm to 350 nm and mass spectra were recorded on a mass spectrometer using alternate-scan positive and negative mode electrospray ionization.

Conditions Used on the LCMS (C)

### Column

The column used is a Waters Acquity BEH HPLC C18, the dimensions of which are 2.1 mm×50 mm. The stationary phase particle size is 1.7 μm.

### Solvents

A: Aqueous solvent=Water+0.05% Formic Acid

B: Organic solvent=Acetonitrile+0.05% Formic Acid

Weak Wash=1:1 Methanol:Water

Strong Wash=Water

### Method

The generic method used has a 2 minute runtime.

| Time/min | % B |
|---|---|
| 0 | 3 |
| 0.1 | 3 |
| 1.5 | 97 |
| 1.9 | 97 |
| 2.0 | 3 |

The above method has a flow rate of 1 mL/min.
The injection volume for the generic method is 0.5 ul
The column temperature is 40° C.
The UV detection range is from 220 to 330 nm

### Conditions Used on the LCMS (D)

### Column

The column used is a Waters Atlantis, the dimensions of which are 4.6 mm×50 mm. The stationary phase particle size is 3 μm.

### Solvents

A: Aqueous solvent=Water+0.05% Formic Acid

B: Organic solvent=Acetonitrile+0.05% Formic Acid

### Method

The generic method used has a 5 minute runtime.

| Time/min | % B |
|---|---|
| 0 | 3 |
| 0.1 | 3 |
| 4 | 97 |
| 4.8 | 97 |
| 4.9 | 3 |
| 5.0 | 3 |

The above method has a flow rate of 3 ml/min.
The injection volume for the generic method is 5 ul
The column temperature is 30 deg
The UV detection range is from 220 to 330 nm

### General Chemistry Section

The intermediates for the preparation of the examples may not necessarily have been prepared from the specific batch of precursor described.

### Description 1: Methyl 4-hydroxy-3-iodobenzoate

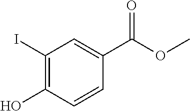

Methyl 4-hydroxybenzoate (23.4 g, 0.154 mol) was dissolved in acetic acid (132 mL) and the solution heated to 65° C. A solution of iodine monochloride (25 g, 0.154 mol) in acetic acid (33 mL) was added dropwise while maintaining temp at 65° C. (oil bath removed during addition). After the addition, the reaction mixture was stirred at 65° C. for 1 h and then cooled to RT and allowed to stand overnight. The resulting precipitate was filtered, washed with petroleum ether and dried to afford the title compound as a light yellow solid (23 g). ¹H NMR (DMSO d-6, 300 MHz) δ ppm: 3.76 (3H, s), 6.95 (1H, d, J=8.0 Hz), 7.79 (1H, dd, J 8.0, 2.0 Hz), 8.20 (1H, d, J=2.0 Hz), 11.30 (1H, br. s).

### Description 2: Methyl 3-bromo-4-hydroxybenzoate

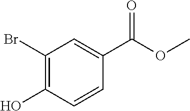

A solution of 3-bromo-4-hydroxybenzoic acid (25 g, 115.74 mmol) in hydrochloric acid/methanol was stirred overnight at RT. Then the resulting solution was concentrated to afford the title compound (28 g). ¹H NMR (CDCl₃, 400 MHz) δ ppm: 3.91 (3H, s), 7.06 (1H, d, J=8.5 Hz), 7.21 (1H, br. s), 7.93 (1H, dd, J 8.5, 1.5 Hz), 8.20 (1H, d, J=1.5 Hz).

### Description 3: Methyl 3-bromo-4-[(1-methylethyl)oxy]benzoate

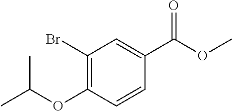

To a solution of methyl 3-bromo-4-hydroxybenzoate (44 g, 192.14 mmol) (Description 2) in acetonitrile (1 L) was added potassium carbonate (79.5 g, 576.4 mmol) and 2-iodopropane (58 mL, 576.4 mmol), the reaction mixture was heated to reflux overnight. The mixture was filtered and the filtrate was concentrated. The residue was partitioned between ethyl acetate and water. The organic phase was separated, washed with water, dried over sodium sulfate and concentrated to afford the title compound (52.4 g). ¹H NMR (CDCl₃, 400 MHz) 5 ppm: 1.41 (6H, d, J=6.5 Hz), 3.89 (3H, s), 4.66 (1H, m), 6.90 (1H, d, J=8.5 Hz), 7.94 (1H, dd, J 8.5, 2.0 Hz), 8.23 (1H, d, J=2.0 Hz).

### Description 4: Methyl 3-cyano-4[(1-methylethyl)oxy]benzoate

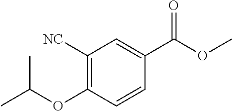

To a solution of methyl 3-bromo-4-[(1-methylethyl)oxy]benzoate (25 g, 91.575 mmol) (Description 3) in DMF (250 mL), under a nitrogen atmosphere, was added zinc cyanide (43 g, 366.3 mmol) and tetrakis(triphenylphosphine)palladium(0) (21 g, 18.32 mmol) and the reaction mixture heated to 120° C. and stirred overnight. After cooling, the mixture was filtered and the filtrate combined with an identical reaction performed on the same scale and DCM (1.5 L) and water (1.5 L) added. The organic phase was separated and the aqueous phase was extracted with DCM (3×300 mL). The combined organic layers were washed with water, dried over sodium sulfate and concentrated. The residue was purified by column chromatography on silica (petroleum ether:ethyl acetate (20:1) to afford the title compound (36 g). ¹H NMR (CDCl₃, 400 MHz) 5 ppm: 1.45 (6H, d, J=6.0 Hz), 3.92 (3H, s), 4.76 (1H, m), 7.00 (1H, d, J 9.0), 8.19 (1H, dd, J 9.0, 2.0 Hz), 8.26 (1H, d, J=2.0 Hz).

### Alternative:

To a solution of methyl 4-hydroxy-3-iodobenzoate (12.5 g, 0.045 mol) (Description 1) in DMF (45 mL) was added sodium cyanide (0.22 g, 0.0045 mar) and copper(I) cyanide (4.48 g, 0.05 mol). The reaction mixture was then flushed with nitrogen and heated at 120° C. for 24 h, cooled to RT, filtered and the filtrate poured into ethyl acetate (200 mL). The filtrate was then washed with iron(III) chloride solution (3×70 mL) (prepared from iron(III) chloride (25 g) in concentrated hydrochloric acid (42 mL) and diluted with 200 mL water). It was then washed with water, dried over sodium sulfate and concentrated to afford a brown solid (12.8 g). This material was then dissolved in DMF (45 mL) and treated with 2-iodopropane (24.6 g, 0.145 mol) and potassium carbonate (28.6 g, 0.207 mol) and the reaction mixture stirred under nitrogen at 90° C. for 12 h. After cooling, the reaction mixture was poured into ice-water (200 mL) and extracted with ethyl acetate (3×70 mL). The organic layers were combined, washed with brine, dried over sodium sulfate and concentrated. The residue was purified by chromatography on silica gel, eluting with petroleum ether:ethyl acetate (30:1) to afford the title compound as a clear oil (3.7 g).

### Description 5: Methyl 3-cyano-4-fluorobenzoate

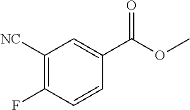

A mixture of 3-cyano-4-fluorobenzoic acid (2.21 g, 13.38 mmol) and p-toluenesulfonic acid monohydrate (0.509 g, 2.68 mmol) in methanol (40 mL) was heated at 90° C. for ca. 18 h. Additional p-toluenesulfonic acid monohydrate (250 mg, 1.31 mmol) was added and heating continued at 80° C. over the weekend. The mixture was then evaporated to dryness and taken up in ethyl acetate (40 mL) and saturated sodium hydrogen carbonate (20 mL). The aqueous layer was separated and further extracted with ethyl acetate (3×15 mL). The combined organics were dried over magnesium sulfate, filtered and evaporated to afford the title compound as a white solid (2.296 g). ¹H NMR (CDCl₃, 400 MHz) δ ppm: 3.96 (3H, s), 7.30 (1H, t, J=9.0 Hz), 8.29 (1H, ddd, J 9.0, 5.0, 2.0 Hz), 8.34 (1H, dd, J 6.0, 2.0 Hz).

### Description 6: 1-Methylethyl 3-cyano-4-[(1-methylethyl)oxy]benzoate

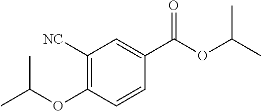

To a solution of isopropanol (1.470 mL, 19.22 mmol) in THF (30 mL) was added dropwise sodium bis(trimethylsilyl)amide (1M in THF) (19.22 mL, 19.22 mmol) under ice bath cooling and the mixture then stirred at RT for 1 h. Methyl 3-cyano-4-fluorobenzoate (Description 5) (2.296 g, 12.82 mmol) was then added and the mixture stirred at 50° C. for ca. 16 h. The reaction mixture was evaporated to near dryness and the residue purified by chromatography on silica gel, eluting with a gradient of 0-100% ethyl acetate in isohexane to afford the title compound (326 mg) ¹H NMR (CDCl₃, 400 MHz) δ ppm: 1.36 (6H, d, J=6.5 Hz), 1.43 (1H, d, J=6.0 Hz), 4.74 (1H, spt, J=6.0 Hz), 5.23 (1H, spt, J=6.0 Hz), 6.98 (1H, d, J=9.0 Hz), 8.17 (1H, dd, J 9.0, 2.0 Hz), 8.23 (1H, d, J=2.0 Hz).

### Description 7: 3-Cyano-4-[(1-methylethyl)oxy]benzoic acid

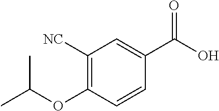

To a mixture of methyl 3-cyano-4-[(1-methylethyl)oxy]benzoate (35 g, 159.8 mmol) (Description 4) in isopropanol (500 mL) was added 2N sodium hydroxide (240 mL) and the reaction mixture was stirred at RT overnight. Water (500 mL) was added to the resulting solution and the isopropanol was removed in vacuo. The residual aqueous phase was washed with ethyl acetate. Then, the aqueous phase was brought to pH 4-5 by 2N hydrochloric acid and the resultant precipitate was filtered and dried to afford the title compound (27.6 g). ¹H NMR (CDCl₃, 400 MHz) 5 ppm: 1.45 (6H, d, J=6.0 Hz), 4.77 (1H, m), 7.02 (1H, d, J=9.0 Hz), 8.24 (1H, dd, 9.0, 2.0 Hz), 8.31 (1H, d, J=2.0 Hz).

### Alternative:

To a mixture of methyl 3-cyano-4-[(1-methylethyl)oxy]benzoate (27 g, 123.28 mmol) (Description 4) in methanol (150 mL) was added sodium hydroxide (5N) (27 mL, 135 mmol) and the mixture stirred at RT for 16 h. The mixture was then reduced in vacuo and acidified with hydrochloric acid (2M) (35 mL). Ethyl acetate (200 mL) was added, the layers separated and the organic layer dried over sodium sulfate and then reduced to a solid which was washed with pentane to afford the title compound (25.27 g).

### Alternative:

A mixture of 1-methylethyl 3-cyano-4-[(1-methylethyl)oxy]benzoate (326 mg, 1.318 mmol) (Description 6) and 2M aqueous sodium hydroxide (0.791 mL, 1.582 mmol) in isopropanol (10 mL) was stirred at RT overnight. The reaction mixture was evaporated to dryness, diluted with water (15 mL), adjusted to ca. pH 2 with 1M aqueous hydrochloric acid then extracted with ethyl acetate (3×15 mL). The combined organics were dried over magnesium sulfate, filtered, and evaporated to afford the title compound as a white solid (267 mg).

### Description 8: Methyl 3-chloro-4-[(1-methylethyl)oxy]benzoate

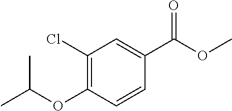

A 5 L flask with an overhead stirrer was charged with methyl 3-chloro-4 hydroxybenzoate (300 g, 1608 mmol), and dry DMF (3000 mL) was poured into the vessel followed by the addition of potassium carbonate (444 g, 3216 mmol) and then at a steady rate of 12 mL/min, 2-iodopropane (321 mL, 3216 mmol) was added. The reaction was allowed to stir at 60° C. for 2 h. The reaction mixture was cooled, filtered and the filtrate (3.5 L) including washings was concentrated to remove nearly all the excess DMF using a trolley pump. To the residue was added water (1000 mL), and the mixture extracted into ethyl acetate (1000 mL) which was then washed with 2N sodium carbonate solution followed by brine (3×500 mL). The ethyl acetate layer was dried over sodium sulfate and evaporated to dryness to afford the title compound as a light yellow oil (375 g). LCMS (B) m/z: 229 [M+1]⁺, Rt 1.24 min.

### Description 9: 1-Methylethyl 3-chloro-4-[(1-methylethyl)oxy]benzoate

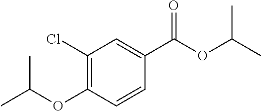

To a suspension of 3-chloro-4-hydroxybenzoic acid hemihydrate (1.5 g, 8.69 mmol) and potassium carbonate (3.60 g, 26.1 mmol) in DMF (22.06 mL), was added 2-iodopropane (2.169 mL, 21.73 mmol). The reaction mixture was heated at 70° C. overnight (ca. 18 h) and then cooled to RT and filtered. The filtrate was diluted with ethyl acetate (20 mL) and water (20 mL) and the aqueous layer separated and extracted with ethyl acetate (3×30 mL). The combined organic layers were dried over a phase separating column and concentrated in vacuo to afford the title compound as a pale yellow oil (2.115 g). LCMS (A) m/z: 256 [M+1]⁺, Rt 1.49 min (acidic).

### Description 10: 3-Chloro-4[(1-methylethyl)oxy]benzoic acid

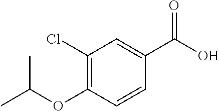

To 1-methylethyl 3-chloro-4-[(1-methylethyl)oxy]benzoate (Description 9) (2.115 g, 8.24 mmol) in ethanol (20 mL) was added 2M sodium hydroxide (8.24 mL, 16.48 mmol) and the reaction mixture was stirred at 60° C. for 2 h. The mixture was then cooled to RT and the solvent removed in vacuo. The resulting white residue was diluted with water (20 mL) and acidified to pH 5 with glacial acetic acid. The resulting white precipitate filtered and dried at 50° C. over 6 h to afford the title compound as a white solid (1.593 g). ¹H NMR (CDCl₃, 400 MHz) δ ppm: 1.43 (6H, d, J 6.0 Hz), 4.69 (1H, spt, J=6.0 Hz), 6.97 (1H, d, J=8.5 Hz), 7.96 (1H, dd, J 8.5, 2.0 Hz), 8.12 (1H, d, J=2.0 Hz).

### Alternative:

Methyl 3-chloro-4-[(1-methylethyl)oxy]benzoate (100 g, 437 mmol) (Description 8) was taken up in a mixture of reagent methanol (555 mL) and 2N sodium hydroxide solution (555 mL). An insoluble white suspension was observed but with time complete solubility was observed. The reaction mixture was stirred at RT overnight and the methanol then removed in vacuo. The resulting basic aqueous solution was acidified to pH 1-2 and the resultant white solid was filtered off, washed with water (500 mL), dried in the vacuum oven overnight to afford the title compound (82 g).

### Description 11: 3-Chloro-4[(1-methylethyl)oxy]benzoyl chloride

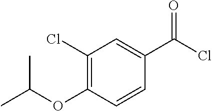

A round bottom flask was charged with 3-chloro-4-[(1-methylethyl)oxy]benzoic acid (22.58 g, 105 mmol) (Description 10), DCM (350 mL) and oxalyl chloride (18.35 mL, 210 mmol). The reaction mixture was cooled to 0° C. in an ice/water bath prior to the addition of DMF (0.350 mL). The solution was allowed to warm to ambient temperature overnight. Volatiles were removed in vacuo to give a cream coloured oil that solidified on cooling to afford title compound as a cream solid (25 g). ¹H NMR (CDCl₃, 400 MHz) δ ppm: 1.44 (6H, d, J=6.0 Hz) 4.73 (1H, spt, J=6.0 Hz) 6.98 (1H, d, J=9.0 Hz) 8.00 (dd, J 9.0, 2.5 Hz), 8.14 (1H, d, J=2.5 Hz).

### Description 12: 1-Methylethyl 5-chloro-6-[(1-methylethyl)oxy]-3-pyridinecarboxylate

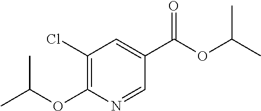

This reaction was performed in a fume hood without lights and the reaction vessel wrapped in aluminum foil. To a solution of 5-chloro-6-hydroxynicotinic acid (4.996 g, 28.8 mmol) in chloroform (170 mL) was added silver carbonate (18.929 g, 68.6 mmol) and 2-iodopropane (11.52 mL, 115 mmol). The reaction mixture was heated to 40° C. and left to stir overnight under nitrogen. The reaction mixture was then cooled to RT and filtered under suction and washed with DCM. The filtrate was reduced in vacuo and the resulting yellow oil dried under high vacuum to afford the title compound (2.56 g). LCMS (A) m/z: 258 [M+1]⁺, Rt 1.47 min (acidic)

### Description 13: 5-Chloro-6-[(1-methylethyl)oxy]-3-pyridinecarboxylic acid

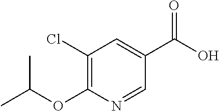

To a solution of 1-methylethyl 5-chloro-6-[(1-methylethyl)oxy]-3-pyridinecarboxylate (2.560 g, 9.93 mmol) (Description 12) dissolved in ethanol (27 mL) was added 2M sodium hydroxide (9.93 mL, 19.87 mmol) and the reaction mixture stirred at 60° C. for 2 h. The reaction mixture was then cooled to RT and concentrated in vacuo. Water (50 mL) was added, the resulting solution acidified to pH 5 with acetic acid and then extracted with ethyl acetate (×3). The organic layers were combined, dried over a phase separator and the solvent removed in vacuo. The residue was dried under high vacuum to afford the title compound as a white solid (1.822 g). LCMS (A) m/z: 214 [M−1]⁻, Rt 0.61 min (basic).

### Description 14: 1-Methylethyl 5-bromo-6-[(1-methylethyl)oxy]-3-pyridinecarboxylate

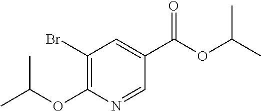

A mixture of 5-bromo-6-hydroxy-3-pyridinecarboxylic acid (4 g, 18.35 mmol), silver carbonate (12.65 g, 45.9 mmol) and 2-iodopropane (7.34 mL, 73.4 mmol) in chloroform (100 mL) was heated at 40° C. in the dark (vessel wrapped with foil) for 15 h. The reaction mixture was then cooled to RT, filtered and the filtrate evaporated in vacuo. The residue was purified on silica gel, eluting with a gradient of 0-30% ethyl acetate in isohexane to afford the title compound as a yellow oil (2.69 g). LCMS (A) m/z: 302/304 [M+1]⁺, Rt 1.51 min (basic).

### Description 15: 1-Methylethyl 5-cyano-6-[(1-methylethyl)oxy]-3-pyridinecarboxylate

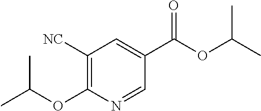

A mixture of 1-methylethyl 5-bromo-6-[(1-methylethyl)oxy]-3-pyridinecarboxylate (605 mg, 2.002 mmol) (Description 14), zinc cyanide (259 mg, 2.202 mmol), tris(dibenzylideneacetone)dipalladium(0) (92 mg, 0.100 mmol) and DPPF (133 mg, 0.240 mmol) in DMF (5 mL) was heated at 120° C. for 15 h. Additional tris(dibenzylideneacetone)dipalladium(0) (92 mg, 0.100 mmol) was added and heating continued at 120° C. for 24 h. A final portion of tris(dibenzylideneacetone)dipalladium(0) (92 mg, 0.100 mmol) was then added and heating continued at 120° C. for another 24 h. The reaction mixture was then cooled to RT, poured into mixture of saturated ammonium chloride, ammonium hydroxide and water (v/v/v: 4:1:4; 70 mL) and then filtered and washed with ethyl acetate. The filtrate was extracted with ethyl acetate (3×10 mL) and the organic layers combined, washed with brine (50 mL), dried over magnesium sulfate, filtered and evaporated in vacuo. Purification by chromatography on silica gel, eluting with a gradient of 0-10% ethyl acetate in isohexane afforded the title compound as a yellow oil (417 mg). LCMS (A) m/z: 249 [M+1]⁺, Rt 1.31 min (basic).

### Description 16: Methyl 5-cyano-6-[(1-methylethyl)oxy]-3-pyridinecarboxylate

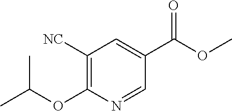

To a solution of methyl 5-cyano-6-oxo-1,6-dihydro-3-pyridinecarboxylate (500 mg, 2.81 mmol) in DMF (5 mL) was added cesium fluoride (1279 mg, 8.42 mmol) and 2-iodopropane (0.842 mL, 8.42 mmol) and the reaction mixture stirred at RT for 64 h. To the reaction mixture was added water (30 mL) which was extracted with ethyl acetate (3×30 mL). The organics were then combined, dried over a phase separation cartridge and volatiles removed by evaporation. Purification by chromatography on silica gel eluting with 0-50% ethyl acetate in isohexane afforded the title compound (220 mg). ¹H NMR (CDCl₃, 400 MHz) δ ppm: 1.45 (6H, d, J 6.0 Hz), 3.96 (3H, s), 5.53 (1H, spt, J=6.0 Hz), 8.46 (1H, d, J=2.0 Hz), 8.96 (1H, d, J=2.0 Hz). In addition, methyl 5-cyano-1-(1-methylethyl)-6-oxo-1,6-dihydro-3-pyridinecarboxylate (265 mg) was also isolated. ¹H NMR (CDCl₃, 400 MHz) δ ppm: 1.46 (6H, d, J=7.0 Hz), 3.93 (3H, s), 5.25 (1H, spt, J=7.0 Hz), 8.33 (1H, d, J=2.5 Hz), 8.42 (1H, d, J=2.5 Hz).

### Description 17: 5-Cyano-6-[(1-methylethyl)oxy]-3-pyridinecarboxylic acid

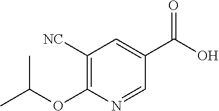

To a suspension of methyl 5-cyano-6-[(1-methylethyl)oxy]-3-pyridinecarboxylate (1.010 g, 4.59 mmol) (Description 16) in isopropanol (22 mL) was added 2M aqueous sodium hydroxide (2.87 ml, 5.73 mmol) and the reaction mixture stirred at RT, under nitrogen, for 1 h 40 min. The solvent was then removed in vacuo and the residue dissolved in water (30 mL), acidified with acetic acid to pH 3 and the mixture extracted with ethyl acetate (3×30 mL). The organic layers were combined, washed with water (50 mL), dried over a phase separator and the solvent removed in vacuo to afford a solid which was dried under high vacuum to afford the title compound as a white solid (911 mg). LCMS (A) m/z: 206 [M+1]⁺, Rt 1.04 min (acidic).

### Alternative:

To a stirred solution of 1-methylethyl 5-cyano-6-[(1-methylethyl)oxy]-3-pyridinecarboxylate (403 mg, 1.623 mmol) (Description 15) in ethanol (5 mL), was added 2M aqueous sodium hydroxide (1.7 mL, 3.40 mmol). The reaction mixture was stirred at 35° C. for 1 h and then cooled to RT and the ethanol removed in vacuo. The resulting suspension was treated with water (10 mL) and the aqueous phase washed with ethyl acetate (10 mL). The aqueous phase was then acidified with 1M aqueous hydrochloric acid and extracted with ethyl acetate (3×25 mL). The combined organic extracts were dried over a phase separating column and concentrated to title compound as a white solid (321 mg).

### Description 18: 4-Amino-2-ethylbenzonitrile

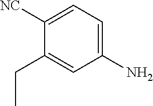

Under ice-bath cooling, iron powder (2.023 g, 36.2 mmol) was added portion-wise (over a period of 5 min) to a solution containing 2-ethyl-4-nitrobenzonitrile (1.666 g, 9.46 mmol) and concentrated hydrochloric acid (5.05 mL, 166 mmol) in ethanol (50 mL). The mixture was heated at 95° C. for 3 h and then cooled and filtered. The filtrate was reduced, diluted with water (40 mL), adjusted to Ca. pH 12 with 50% aqueous sodium hydroxide and extracted with ethyl acetate (4×30 mL). The organics were combined, dried over magnesium sulfate, filtered and reduced to afford the title compound as a dark brown solid (1.3966 g). LCMS (A) m/z: 147 [M+1]⁺, Rt 0.96 min (acidic).

### Description 19: 4-Amino-2-ethyl-5-iodobenzonitrile

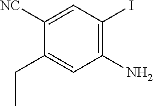

N-Iodosuccinimide (2.149 g, 9.55 mmol) was added to a solution of 4-amino-2-ethylbenzonitrile (1.3966 g, 9.55 mmol) (Description 18) in acetic acid (13 mL) and the mixture stirred at RT for 2 h. The mixture was reduced to near dryness then diluted with ethyl acetate (50 mL) and sodium thiosulfate solution (20 mL), followed by sodium hydroxide (1M) (20 mL). The aqueous was separated and extracted further with ethyl acetate (2×20 mL) and the organic layers combined, dried over magnesium sulfate, filtered and reduced to afford the title compound as a dark brown solid (2.6 g). LCMS m/z: 273 [M+1]⁺, Rt 1.17 min (acidic). ¹H NMR (CDCl₃, 400 MHz) δ ppm: 1.26 (3H, t, J=7.5 Hz), 2.73 (2H, q, J=7.5 Hz), 6.60 (1H, s), 7.83 (1H, s). The following peaks indicate that the sample also contains ca. 10-15% of 4-amino-2-ethyl-3-iodobenzonitrile δ ppm: 3.05 (q, J=7.5 Hz), 6.57 (d, J=8.5 Hz), 7.35 (d, J=8.5 Hz).

### Description 20: 4-Amino-2-ethyl-5-methylbenzonitrile

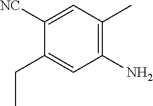

A mixture of 4-amino-2-ethyl-5-iodobenzonitrile (1.5548 g, 5.71 mmol) (Description 19), methyl boronic acid (0.866 g, 14.47 mmol), cesium carbonate (7.45 g, 22.86 mmol) and 1,1′-bis(diphenylphosphino)ferrocenedichloro palladium(II) dichloromethane complex (0.460 g, 0.629 mmol) in 1,4-dioxane (15 mL) was degassed with nitrogen for 15 min, then refluxed at 95° C. overnight. The mixture was cooled then filtered through celite. The filtrate was reduced and the residue purified by chromatography on silica gel, eluting with a gradient of 0-40% ethyl acetate in isohexane to afford the title compound as an orange solid (580 mg). LCMS (A) m/z: 161 [M+1]⁺, Rt 1.05 min (acidic).

### Alternative:

A mixture of 4-amino-2-ethyl-5-iodobenzonitrile (1.2693 g, 4.67 mmol) (Description 19), methyl boronic acid (0.670 g, 11.20 mmol), cesium carbonate (6.08 g, 18.66 mmol) and 1,1′-bis(diphenylphosphino)ferrocene-palladium(II) dichloride dichloromethane complex (0.381 g, 0.467 mmol) in 1,4-dioxane (20 mL) was degassed with nitrogen for 15 min, then heated in a microwave at 120° C. for 4 h. The mixture was then cooled and filtered through a celite pad and the filtrate reduced. Purification by chromatography on silica gel, eluting with a gradient of 0-30% ethyl acetate in cyclohexane afforded the title compound as a brown solid (0.5511 g).

### Description 21: 6-Ethyl-1H-indazole-5-carbonitrile

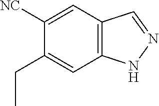

A solution of 4-amino-2-ethyl-5-methylbenzonitrile (580 mg, 3.62 mmol) (Description

20) in chloroform (16 mL) was added acetic anhydride (0.683 mL, 7.24 mmol), potassium acetate (373 mg, 3.80 mmol), 18-crown-6 (191 mg, 0.724 mmol) and the resulting mixture stirred at RT for 15 min. t-Butyl nitrite (0.903 mL, 7.60 mmol) was then added and the mixture heated at 71° C. overnight. The reaction mixture was cooled and then stirred with saturated aqueous sodium bicarbonate solution (ca. 15 mL). The aqueous layer was separated and further extracted with DCM (3×15 mL) and the organics combined and reduced. The residue was then taken up in methanol (5 mL) treated with hydrochloric acid 6M aqueous solution (10.86 mL, 65.2 mmol) and the mixture heated at 50° C. for 1 h then reduced to near dryness. The residue was then diluted with ethyl acetate (30 mL), washed with water (10 mL), reduced, then purified by chromatography on silica gel, eluting with a gradient 0-40% ethyl acetate in isohexane to afford the title compound as a yellow solid (240 mg). LCMS (A) m/z: 172 [M−1]⁺, Rt 0.95 min (acidic).

### Description 22: 1-Acetyl-6-ethyl-1H-indazole-5-carbonitrile

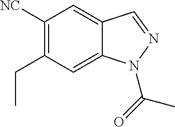

To a solution of 4-amino-2-ethyl-5-methylbenzonitrile (1.1311 g, 7.06 mmol) (Description 20) in chloroform (20 mL) was added sequentially acetic anhydride (1.332 mL, 14.12 mmol), potassium acetate (0.728 g, 7.41 mmol), a solution of 18-crown-6 (0.373 g, 1.412 mmol) in chloroform (5 mL), and finally t-butyl nitrite (2.050 mL, 15.53 mmol). The mixture was then heated at 75° C. for ca. 20 h. The dark brown mixture was cooled then stirred with saturated aqueous sodium bicarbonate solution (25 mL) for ca. 5 min. The aqueous layer was separated and extracted with DCM (2×20 mL) and the organics combined, reduced, then purified by chromatography on silica gel, eluting with a gradient of 0-20% ethyl acetate in cyclohexane to afford the title compound as a orange solid (573 mg). LCMS (A) m/z: 172 [M-COMe+1]⁺, Rt 1.32 min (acidic).

### Description 23: 4-Amino-5-iodo-2-(methyloxy)benzonitrile

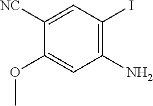

A mixture of 2-(methyloxy)-4-nitrobenzonitrile (1461 mg, 8.20 mmol), iron powder (1754 mg, 31.4 mmol) and concentrated hydrochloric acid solution (4.3 mL, 142 mmol) in ethanol (45 mL) was heated at 100° C. for ca. 2 h. The mixture was cooled then filtered and the filtrate reduced, diluted with water (40 mL), adjusted to ca. pH 12 with 50% sodium hydroxide solution and extracted with ethyl acetate (4×30 mL). The organics were combined, dried over magnesium sulfate, filtered and reduced to afford 4-amino-2-(methyloxy)benzonitrile as a dark brown solid (937 mg). This material was taken up in acetic acid (12 mL) and N-iodosuccinimide (1422 mg, 6.32 mmol) added. The mixture was stirred at RT for 3 h then reduced to near dryness and the residue diluted with ethyl acetate (50 mL) and sodium thiosulfate solution (20 mL), followed by 1M sodium hydroxide solution (20 mL). The aqueous was separated and extracted further with ethyl acetate (2×20 mL) and the organics combined, dried over magnesium sulfate, filtered and reduced to afford the title compound as a dark brown solid (1.8691 g). LCMS (A) m/z: 275 [M+1]⁺, Rt 1.04 min (acidic).

### Description 24: 4-Amino-5-methyl-2-(methyloxy)benzonitrile

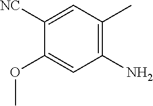

A mixture of 4-amino-5-iodo-2-(methyloxy)benzonitrile (1.8691 g, 6.82 mmol) (Description 23), methyl boronic acid (1.225 g, 20.46 mmol), cesium fluoride (4.14 g, 27.3 mmol) and 1,1′-bis(diphenylphosphino)ferrocenedichloro palladium(II) dichloromethane complex (0.4 g, 0.547 mmol) in dioxane (20 mL) was degassed with nitrogen for 15 min was then refluxed at 95° C. overnight. The mixture was then cooled, filtered through a celite pad and the filtrate reduced. The residue was then diluted with ethyl acetate (50 mL) and water (15 mL) and the aqueous separated and further extracted with ethyl acetate (4×15 mL). The organics were combined and reduced and the residue purified by chromatography on silica gel, eluting with a gradient of 0-100% ethyl acetate in isohexane to afford the title compound as a brown solid (858 mg). LCMS (A) m/z: 163 [M+1]⁺, Rt 0.91 min (basic).

### Description 25: 6-(Methyloxy)-1H-indazole-5-carbonitrile

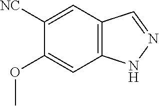

A solution of 4-amino-5-methyl-2-(methyloxy)benzonitrile (858 mg, 5.29 mmol) (Description 24) in chloroform (25 mL) was added acetic anhydride (0.998 mL, 10.58 mmol), then potassium acetate (545 mg, 5.55 mmol) and 18-crown-6 (280 mg, 1.058 mmol) and the mixture stirred at RT for 15 min. 1-butyl nitrite (1.320 mL, 11.11 mmol) was added and the mixture refluxed at 75° C. overnight. The reaction mixture was reduced to near dryness, taken up in ethanol (25 mL) and potassium hydroxide (4452 mg, 79 mmol) added and the mixture stirred at RT for 5 h. The solvent was then reduced and the residue diluted with ethyl acetate (60 mL) and washed with water (20 mL). The aqueous was further extracted with DCM (5×20 mL) and the organic layers combined, reduced then purified by chromatography on silica gel, eluting with a gradient of 0-100% ethyl acetate in isohexane to afford the title compound as a dark brown solid (116 mg). LCMS (A) m/z: 174 [M+1]⁺, Rt 0.80 min (acidic).

### Description 26: 1-Acetyl-5-bromo-6-fluoro-1H-indazole

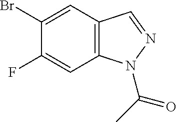

Acetic anhydride (1.488 mL, 15.77 mmol) was added to a suspension of 4-bromo-5-fluoro-2-methylaniline (1.6087 g, 7.88 mmol) in chloroform (18 mL) under ice-bath cooling and the mixture stirred at RT for 5 min. Potassium acetate (0.812 g, 8.28 mmol), a solution of 18-crown-6 (0.417 g, 1.577 mmol) in chloroform (4 mL) and then t-butyl nitrite (2.186 mL, 16.56 mmol) were then added and the mixture heated at 75° C. for 16 h. The dark brown mixture was then cooled, DCM (20 mL) added and the organic layer washed with saturated aqueous sodium bicarbonate solution (ca. 20 mL) then reduced. The residue was purified by chromatography on silica gel eluting with a gradient of 0-30% ethyl acetate in hexane. Impure fractions were purified further by chromatography on silica gel eluting with a gradient of 0-20% ethyl acetate in isohexane. All pure fractions were combined and then reduced to afford the title compound as a light orange solid (984 mg). ¹H NMR (CDCl₃, 400 MHz) δ ppm: 2.79 (3H, s), 7.95 (1H, dd, 6.5 Hz, 0.5 Hz), 8.07 (1H, d, J=1.0 Hz), 8.25 (1H, dd, J 9.0, 0.5 Hz).

### Description 27: 6-Fluoro-1H-indazole-5-carbonitrile

### and

### Description 28: 5-Bromo-6-fluoro-1H-indazole

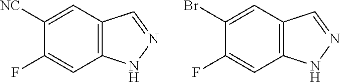

A suspension of 1-acetyl-5-bromo-6-fluoro-1H-indazole (984 mg, 3.83 mmol) (Description 26), zinc cyanide (449 mg, 3.83 mmol), and tetrakis(triphenylphosphine)palladium(0) (442 mg, 0.383 mmol) in DMF (15 mL) was degassed with nitrogen for 15 min then heated at 120° C. for 16 h. Additional tetrakis(triphenylphosphine)palladium(0) (442 mg, 0.383 mmol) and DMF (6 mL) were added and the mixture heated at 130° C. for a further 20 h. The solution was cooled and ethyl acetate (ca. 60 mL) and saturated aqueous sodium bicarbonate solution (ca. 40 mL) added. The aqueous was separated and further extracted with ethyl acetate (3×20 mL) and the organics combined, reduced and purified by chromatography on silica gel eluting with a gradient of 0-80% ethyl acetate in isohexane to afford 6-fluoro-1H-indazole-5-carbonitrile as an off-white solid (246 mg). LCMS (A) m/z: 162 [M+1]⁺, Rt 0.82 min (acidic). In addition, 5-Bromo-6-fluoro-1H-indazole was isolated as a pale yellow solid (384 mg). LCMS (A) m/z: 215/217 [M+1]⁺, Rt 1.02 min (acidic).

### Alternative Preparation of 6-Fluoro-1H-indazole-5-carbonitrile (Description 27)

A suspension of 5-bromo-6-fluoro-1H-indazole (384 mg, 1.786 mmol) (Description

28) and zinc cyanide (252 mg, 2.143 mmol) in DMF (12 mL) was degassed with nitrogen for 15 min. Tetrakis(triphenylphosphine)palladium(0) (413 mg, 0.357 mmol) was then added and the solution degassed with nitrogen for an additional 15 min then heated at 115° C. for ca. 90 h. The solution was then cooled and ethyl acetate (ca. 40 mL) and saturated aqueous sodium bicarbonate solution (20 mL) added. The aqueous was separated and further extracted with ethyl acetate (3×15 mL) and the organics combined, reduced and purified by chromatography on silica gel eluting with a gradient of 0-80% ethyl acetate in isohexane to afford the title compound as a white solid (218 mg).

### Description 29: 1H-Indazole-5-carbonitrile

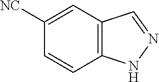

A mixture of 4-fluoro-3-formylbenzonitrile (3.45 g, 23.14 mmol), hydrazine hydrate (32.6 mL, 370 mmol) and potassium carbonate (3.52 g, 25.4 mmol) in ethanol (12 mL) was heated at 50° C. overnight (ca. 18 h). The mixture was then cooled and diluted with saturated aqueous ammonium chloride solution (ca. 60 mL) and ethyl acetate (60 mL). The aqueous layer was separated and further extracted with ethyl acetate (5×40 mL) and the organics combined, dried over magnesium sulfate, filtered and reduced. The resulting residue was triturated with minimal methanol, ethyl acetate and ether (ca. 15 mL) then filtered. The solid obtained was dried in the vacuum oven to afford the title compound as a pink solid (1.906 g). LCMS (A) m/z: 144 [M+1]⁺, Rt 0.80 min (acidic). The filtrate was reduced and dried to afford some impure title compound as a reddish pink solid (1.39 g).

### Description 30:1-Acetyl-1H-indazole-4-carbonitrile

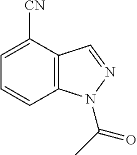

1H-indazole-4-carbonitrile (10 g, 70 mmol) was added to a solution of pyridine (5.75 g, 73 mmol), acetic anhydride (7.85 g, 77 mmol) and 4-dimethylaminopyridine (0.9 g, 7 mmol) in THF (120 mL) and the mixture stirred at RT for 2.5 h. The mixture was then concentrated in vacuo and DCM added. This mixture was washed with 1M aqueous hydrochloric acid solution (×2) and then concentrated to afford the title compound as a solid (11 g). LCMS m/z: 186 [M+1]⁺, Rt 1.435 min.

### Description 31: 4-Bromo-1H-indazole

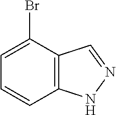

3-Bromo-2-methylaniline (50 g, 269 mmol) in chloroform (670 mL) was cooled in an ice-bath to 5° C. with overhead stirring. Acetic anhydride (57.6 mL, 610 mmol) was added rapidly drop-wise via dropping funnel over ca. 5 min giving a brown homogeneous solution. The ice-bath was removed and the reaction mixture allowed to warm to RT. Potassium acetate (7.65 g, 78 mmol) was then added (at 22° C.) followed by rapid, drop-wise addition of isoamyl nitrite (78 mL, 578 mmol). On complete addition the temperature remained at ca. 22° C., and a heavy, flocculent precipitate formed. The reaction mixture was stirred at RT for ca. 1 h then heated to 68° C., (external) which maintained an internal temperature of 62° C., without vigorous refluxing. At this temperature the solid re-dissolved and the temperature was maintained at 68° C. overnight. The reaction mixture was allowed to cool to RT and was then concentrated in vacuo giving a dark brown oil. Water (500 mL) was added to the mixture which was re-reduced to an orange/brown solid which was treated with concentrated hydrochloric acid (300 mL) and heated to 50° C. (internal temp.) for 2 h. After 2 h, the heat was removed and the reaction mixture cooled to RT then cooled further in an acetone/dry-ice bath to (initially) ca. 0° C. Aqueous sodium hydroxide (10M) was then added to basify the mixture to pH 10, maintaining the internal temperature between 20-30° C. The resultant buff coloured solid/liquid mixture was then filtered and the solid washed with water (3×250 mL), to afford the title compound which was allowed to air dry under suction, then transferred to a crystallizing dish and dried in vacuo at ca. 50° C. overnight (50.9 g). LCMS (B) m/z: 197/199 [M+1]⁺, Rt 0.92 min.

### Description 32: 5-Bromo-6-methyl-1H-indazole

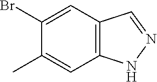

Acetic anhydride (1.415 mL, 14.99 mmol) was added to a solution of 4-bromo-2,5-dimethylaniline (1.5 g, 7.50 mmol) in chloroform (12 mL) under ice-bath cooling and the mixture was then stirred at RT for ca. 5 min. Potassium acetate (0.773 g, 7.87 mmol) was then added and the mixture stirred at RT for 5 min. A solution of 18-crown-6 (0.396 g, 1.499 mmol) in chloroform (7 mL) was added followed by t-butyl nitrite (2.177 mL, 16.49 mmol). The resulting mixture was refluxed at 75° C. for 18 h. The resulting dark brown mixture was cooled, washed with saturated aqueous sodium bicarbonate solution (50 mL) then reduced. The resulting residue was taken up in methanol (5 mL) and concentrated hydrochloric acid (10 mL, 122 mmol) then heated at 50° C. for 2 h. The mixture was then reduced to dryness and purified by chromatography on silica gel, eluting with a gradient of 0-70% ethyl acetate in hexane to afford the title compound as a yellow solid (1.1152 g). LCMS (A) m/z: 211/213 [M+1]⁺, Rt 1.03 min (acidic).

### Description 33: 1-Acetyl-5-bromo-6-methyl-1H-indazole

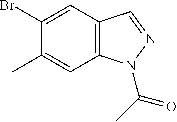

To a solution of 4-bromo-2,5-dimethylaniline (1.8037 g, 9.02 mmol) in chloroform (20 mL) was added sequentially acetic anhydride (1.701 mL, 18.03 mmol), potassium acetate (0.929 g, 9.47 mmol), a solution of 18-crown-6 (0.477 g, 1.803 mmol) in chloroform (5 mL) and t-butyl nitrite (2.62 mL, 19.83 mmol) and the mixture was refluxed at 75° C. for ca. 20 h. The dark brown reaction mixture was cooled then stirred with saturated aqueous sodium bicarbonate solution (25 mL) for 5 min and the aqueous then separated and extracted with DCM (2×20 mL). The organics were combined, evaporated and then purified by chromatography on silica gel, eluting with a gradient of 0-15% ethyl acetate in cyclohexane to afford the title compound as a light orange solid (1.85 g). LCMS (A) m/z: 253 [M+1]⁺, Rt 1.33 min (acidic).

### Description 34: 1,1-Dimethylethyl 5-bromo-6-methyl-1H-indazole-1-carboxylate

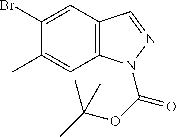

To a solution of 5-bromo-6-methyl-1H-indazole (386 mg, 1.829 mmol) (Description 32) in acetonitrile (18 mL), triethylamine (0.382 mL, 2.74 mmol) and 4-dimethylaminopyridine (11.17 mg, 0.091 mmol) was added at 0° C. After stirring for 10 min, di-tert-butyl dicarbonate (0.510 mL, 2.195 mmol) was added drop-wise. After stirring for 15 min, the reaction mixture was allowed to warm to RT and was stirred overnight. The solvent was removed under reduced pressure and the residue purified by chromatography on silica gel eluting with a gradient of 0-20% ethyl acetate in isohexane to afford the title compound as a pale yellow solid (415 mg). LCMS (A) m/z: 311/313 [M+1]⁺, Rt 1.42 min (acidic). In addition, 1,1-dimethylethyl 5-bromo-6-methyl-2H-indazole-2-carboxylate (117 mg) was also isolated as a pale yellow solid.

### Description 35: 6-Methyl-1H-indazole-5-carbonitrile

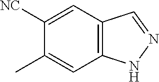

A mixture of 1,1-dimethylethyl 5-bromo-6-methyl-1H-indazole-1-carboxylate (399 mg, 1.282 mmol) (Description 34), zinc cyanide (151 mg, 1.282 mmol) and tetrakis(triphenylphosphine)palladium(0) (148 mg, 0.128 mmol) in DMF (12 mL) was degassed with nitrogen for 10 min and then heated at 120° C. for 20 h under nitrogen. After cooling to RT, the reaction mixture was poured into saturated aqueous sodium bicarbonate solution (70 mL) and extracted with ethyl acetate (2×10 mL). The organic layers were combined, washed with brine (50 mL), dried over sodium sulfate, filtered and reduced in vacuo. The residue was triturated with ethyl acetate/isohexane (1:5; 2×10 mL) and the resulting solid collected by filtration, washing with additional ethyl acetate/isohexane (1:5), to afford the title compound as a white solid (157 mg). LCMS (A) m/z: 158 [M+1]⁺, Rt 0.84 min (basic).

### Description 36: 5-Bromo-4-methyl-1H-indazole

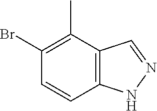

Acetic anhydride (1.148 mL, 12.17 mmol) was added to a suspension of 4-bromo-2,3-dimethylaniline (1.2177 g, 6.09 mmol) in chloroform (12 mL) under ice-bath cooling then stirred at RT for 5 min. Potassium acetate (0.627 g, 6.39 mmol) was added followed by a solution of 18-crown-6 (0.322 g, 1.217 mmol) in chloroform (4 mL). The mixture was then stirred at RT for 10 min then t-butyl nitrite (1.767 mL, 13.39 mmol) was added. The resulting mixture was refluxed at 75° C. for 16 h and then cooled. DCM (20 mL) was added and the mixture washed with saturated aqueous sodium bicarbonate solution (20 mL) then reduced. The resulting residue was taken up in methanol (5 mL) and concentrated hydrochloric acid (8.00 mL, 97 mmol) and then heated at 50° C. for 2 h. The mixture was then reduced to dryness and partitioned between ethyl acetate (60 mL) and water (20 mL). The aqueous layer was separated, extracted with DCM (5×10 mL) and the organics combined and reduced. The resulting residue was then purified by chromatography on silica gel, eluting with a gradient of 0-70% ethyl acetate in hexane to afford the title compound as a light brown solid (969 mg). LCMS (A) m/z: 211/213 [M+1]⁺, Rt 1.06 min (acidic).

### Description 37: 1-Acetyl-5-bromo-6-chloro-1H-indazole

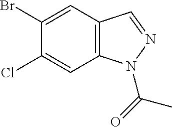

Under ice-bath cooling, acetic anhydride (1.553 mL, 16.46 mmol), was added to a solution of 4-bromo-5-chloro-2-methylaniline (1.815 g, 8.23 mmol) in chloroform (20 mL). Potassium acetate (0.889 g, 9.05 mmol), a solution of 18-crown-6 (0.435 g, 1.646 mmol) in chloroform (5 mL) and then t-butyl nitrite (2.282 mL, 17.29 mmol) were then added sequentially and the mixture was refluxed at 78° C. for 16 h. The reaction mixture was cooled and stirred (under ice bath cooling) with saturated aqueous sodium bicarbonate solution (20 mL). The aqueous layer was separated and extracted with DCM (3×20 mL) and the organics combined and reduced. The resulting residue was purified twice by chromatography on silica gel, eluting with a gradient of 0-20% ethyl acetate in isohexane to afford the title compound as an orange solid (1.52 g). ¹H NMR (CDCl₃, 400 MHz) 3 ppm: 2.79 (3H, s), 8.03 (1H, s), 8.06 (1H, d, J=1 Hz), 8.64 (1H, m).

### Description 38: 6-Chloro-1H-indazole-5-carbonitrile

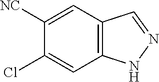

A mixture of 1-acetyl-5-bromo-6-chloro-1H-indazole (405 mg, 1.481 mmol) (Description 37) and zinc cyanide (87 mg, 0.740 mmol) in DMF (12 mL) was degassed with nitrogen for 15 min followed by addition of tetrakis(triphenylphosphine)palladium(0) (257 mg, 0.222 mmol), degassed for a further 15 min and then heated at 120° C. for ca. 16 h. The solution was cooled and partitioned between ethyl acetate (ca. 40 mL) and saturated aqueous sodium bicarbonate solution (ca. 20 mL). The aqueous layer was separated and further extracted with ethyl acetate (3×15 mL) and the organics combined, dried (phase separator) and reduced to afford the title compound as a brown solid (579 mg) which was used directly without purification by chromatography. LCMS (A) m/z: 178 [M+1]⁺, Rt 0.90 min (acidic).

### Description 39: Ethyl 4-(5-cyano-6-ethyl-1H-indazol-1-yl)butanoate

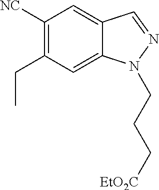

A mixture of 1-acetyl-6-ethyl-1H-indazole-5-carbonitrile (573 mg, 2.69 mmol) (Description 22) and concentrated hydrochloric acid (0.552 mL, 6.72 mmol) in methanol (15 mL) was heated at 80° C. for 2 h. The resulting solution was reduced to dryness, diluted with DMF (15 mL), and potassium carbonate (1857 mg, 13.44 mmol) added followed by ethyl 4-bromobutanoate (0.567 mL, 3.76 mmol). The resulting mixture was heated at 80° C. for 2 h then cooled and filtered. The filtrate was reduced and the residue purified by chromatography on silica gel, eluting with a gradient of 0-40% ethyl acetate in cyclohexane to afford the title compound as an orange oil (471 mg). LCMS (A) m/z: 286 [M+1]⁺, Rt 1.25 min (acidic). In addition, ethyl 4-(5-cyano-6-ethyl-2H-indazol-2-yl)butanoate (230 mg) was also isolated.

### Description 40: Ethyl 3-(4-bromo-1H-indazol-1-yl)propanoate

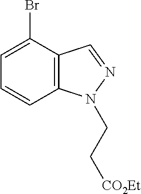

4-Bromo-1H-indazole (48.9 g, 248 mmol) (Description 31) in acetonitrile (1400 mL) was treated with ethyl acrylate (33.6 mL, 310 mmol) followed by DBU (18.70 mL, 124 mmol), and the reaction mixture heated to 70° C. for 3 h. Another portion of DBU (18.70 mL, 124 mmol) was added and the heating continued overnight. The reaction mixture was concentrated in vacuo, re-dissolved in ethyl acetate (1000 mL), washed with water (3×500 mL), brine, and then dried over magnesium sulfate to afford the title compound as a brown oil (71.8 g). LCMS (B) m/z: 297/299 [M+1]⁺, Rt 1.17 min.

### Description 41: Ethyl 4-(5-bromo-6-methyl-1H-indazol-1-yl)butanoate

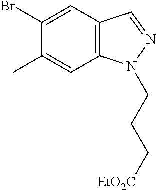

A mixture containing 5-bromo-6-methyl-1H-indazole (600 mg, 2.84 mmol) (Description 32), cesium carbonate (1389 mg, 4.26 mmol), and ethyl 4-bromobutanoate (0.657 mL, 4.55 mmol) in DMF (10 mL) was heated at 80° C. for 4 h. The reaction mixture was reduced to near dryness and partitioned between ethyl acetate (20 mL) and water (10 mL). The aqueous was separated and extracted further with ethyl acetate (3×10 mL) and the organics combined and concentrated. The resulting residue was then purified by chromatography on silica gel, eluting with a gradient of 0-30% ethyl acetate in isohexane to afford the title compound as an orange oil (617 mg). LCMS (A) m/z: 325/327 [M+1]⁺, Rt 1.32 min (acidic). Eluting further, with 100% ethyl acetate, afforded ethyl 4-(5-bromo-6-methyl-2H-indazol-2-yl)butanoate (299 mg).

### Alternative:

A suspension of 1-acetyl-5-bromo-6-methyl-1H-indazole (1.85 g, 7.31 mmol) (Description 33) and concentrated hydrochloric acid (1.501 mL, 18.27 mmol) in methanol (20 mL) was heated at 80° C. for 2 h. The solution was evaporated to dryness, diluted with DMF (15 mL), and treated with potassium carbonate (4 g, 28.9 mmol) then ethyl 4-bromobutanoate (1.5 mL, 9.96 mmol). The resulting mixture was heated at 80° C. for 2 h and then the mixture was cooled and the filtered, washing with ethyl acetate. The filtrate was reduced and the residue purified by chromatography on silica gel, eluting with a gradient of 0-30% ethyl acetate in cyclohexane to afford the title compound as an orange oil (1.4383 g). Eluting further, with 30-100% ethyl acetate in cyclohexane, afforded ethyl 4-(5-bromo-6-methyl-2H-indazol-2-yl)butanoate (804 mg).

### Description 42: Ethyl 4-(5-bromo-4-methyl-1H-indazol-1-yl)butanoate

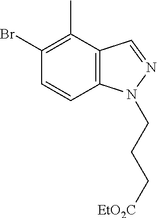

A mixture containing 5-bromo-4-methyl-1H-indazole (969 mg, 4.59 mmol) (Description 36), cesium carbonate (2244 mg, 6.89 mmol), and ethyl 4-bromobutanoate (0.995 mL, 6.89 mmol) in DMF (14 mL) was heated at 80° C. for 2.5 h. The reaction mixture was reduced to near dryness and partitioned between ethyl acetate (30 mL) and water (15 mL), the aqueous separated and extracted further with ethyl acetate (4×10 mL). The organics were combined and reduced and the residue purified by chromatography on silica gel, eluting with a gradient of 0-30% ethyl acetate in isohexane to afford the title compound as an yellow oil (852 mg). LCMS (A) m/z: 325/327 [M+1]⁺, Rt 1.36 min (acidic). Eluting further, with 100% ethyl acetate, afforded ethyl 4-(5-bromo-4-methyl-2H-indazol-2-yl)butanoate (401 mg).

### Description 43: Ethyl 4-(5-bromo-6-chloro-1H-indazol-1-yl)butanoate

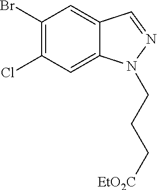

A suspension of 1-acetyl-5-bromo-6-chloro-1H-indazole (1.7336 g, 6.34 mmol) (Description 37) and 6M aqueous hydrochloric acid (0.5 mL, 16.46 mmol) in methanol (20 mL) was heated at 80° C. for 2 h. The dark brown solution was reduced to dryness, diluted with DMF (20 mL), and then treated with potassium carbonate (3.50 g, 25.4 mmol) then ethyl 4-bromobutanoate (1.050 mL, 6.97 mmol). The resulting mixture was heated at 80° C. for 1 h then reduced to dryness and partitioned between brine (20 mL) and ethyl acetate (30 mL). The aqueous was separated and extracted with ethyl acetate (2×15 mL). The organics were combined, reduced, and purified by chromatography on silica gel, eluting with a gradient of 0-30% ethyl acetate in cyclohexane to afford the title compound as an orange-red oil (1.1979 g). LCMS (A) m/z: 345/347 [M+1]⁺, Rt 1.39 min (acidic). In addition, ethyl 4-(5-bromo-6-chloro-2H-indazol-2-yl)butanoate (725 mg) was also isolated.

### Description 44: Ethyl 4-(5-cyano-1H-indazol-1-yl)butanoate

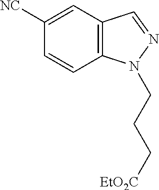

To a solution of 1H-indazole-5-carbonitrile (1.2 g, 8.38 mmol) (Description 29) in DMF (60 mL) was added cesium carbonate (4.10 g, 12.57 mmol) and ethyl 4-bromobutanoate (2.422 mL, 16.77 mmol). The reaction mixture was then heated at 80° C. for 1 h, then left stirring at rt overnight. The mixture was then diluted with ethyl acetate (ca. 100 mL), washed with water (ca. 80 mL) and the aqueous phase re-extracted with ethyl acetate (3×100 mL). The organic phases were combined, dried over magnesium sulfate, filtered and reduced in vacuo. Purification by chromatography on silica gel, eluting with 0-50% ethyl acetate in isohexane afforded the title compound as a colourless oil which (1.24 g). LCMS (A) m/z: 258 [M+1]⁺, Rt 1.06 min (acidic).

### Description 45: Ethyl 3-(4-cyano-1H-indazol-1-yl)propanoate

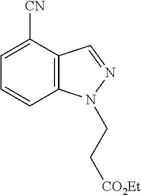

Ethyl 3-(4-bromo-1H-indazol-1-yl)propanoate (35 g, 118 mmol) (Description 40), in dry 1,4-dioxane (450 mL) was de-gassed and flushed with nitrogen. Tetrakis(triphenylphosphine)palladium(0) (6.81 g, 5.89 mmol) was added and the mixture de-gassed and flushed again. Zinc cyanide (13.97 g, 119 mmol) was added and the flushing repeated. The reaction mixture was heated to reflux overnight. The reaction mixture was allowed to cool to RT filtered through celite, washing with ethyl acetate (250 mL). The filtrate was concentrated and re-dissolved in ethyl acetate (500 mL), washed with water (3×250 mL) and brine. The aqueous washes were combined and back extracted with ethyl acetate and the organic layers combined, dried over anhydrous magnesium sulfate, filtered and concentrated. Purification by chromatography on silica gel, eluting with 0-30% ethyl acetate in isohexane afforded the title compound as pale yellow oil which readily crystallized on standing (22.57 g). LCMS (B) m/z: 244 [M+1]⁺, Rt 0.98

### Description 46: 1,1-Dimethylethyl 4-cyano-1H-indazole-1-carboxylate

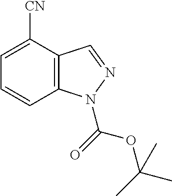

A mixture of 1H-indazole-4-carbonitrile (0.143 g, 0.999 mmol), triethylamine (0.153 mL, 1.099 mmol), 4-dimethylaminopyridine (1.220 mg, 9.99 μmol) and di-tert-butyl dicarbonate (0.464 mL, 1.998 mmol) in DCM (4 mL) was stirred at RT for 1 h. The mixture was then diluted with ethyl acetate (30 mL) and washed with 2M aqueous hydrochloric acid solution (10 mL). The organic layer was then dried over magnesium sulfate, reduced and purified by chromatography on silica gel, eluting with 20% ethyl acetate in hexane to afford the title compound as a white solid (86 mg). ¹H NMR (CDCl₃, 400 MHz) δ ppm: 1.75 (9H, s), 7.63 (1H, m), 7.70 (1H, m), 8.37 (1H, d, J 1.0 Hz), 8.50 (1H, d, J=8.5 Hz). In addition, 1,1-dimethylethyl 4-cyano-2H-indazole-2-carboxylate (91 mg) was also isolated. ¹H NMR (CDCl₃, 400 MHz) δ ppm: 1.75 (9H, s), 7.39 (1H, dd, J 9.0, 7.0 Hz), 7.59 (1H, dd, J 7.0, 1 Hz), 8.03 (1H, dt, J 9.0, 1 Hz), 8.82 (1H, d, J=1.0 Hz).

### Description 47: Ethyl 3-(5-cyano-1H-indazol-1-yl)propanoate

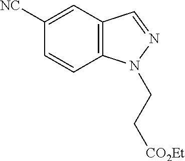

To a solution of 1H-indazole-5-carbonitrile (0.4 g, 2.79 mmol) (Description 29) in DMF (15 mL) was added ethyl 3-bromopropanoate (0.356 mL, 2.79 mmol), cesium carbonate (1.093 g, 3.35 mmol) and the reaction heated to 80° C. for 3 h under nitrogen. The reaction mixture was then reduced to dryness before the addition of ethyl acetate (30 mL). This was then washed using water (30 mL), the organic phase then dried over phase separating cartridge and volatiles removed in vacuo to afford crude material. This was purified by chromatography on silica gel, eluting with a gradient of 0-50% ethyl acetate in isohexane to afford the title compound as orange oil (563 mg). LCMS (A) m/z: 244 [M+1]⁺, Rt 1.10 min (acidic).

### Description 48: Ethyl 4-(5-cyano-6-methyl-1H-indazol-1-yl)butanoate

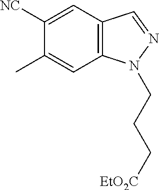

A mixture of ethyl 4-(5-bromo-6-methyl-1H-indazol-1-yl)butanoate (1.4383 g, 4.42 mmol) (Description 41), zinc cyanide (0.467 g, 3.98 mmol) and tetrakis(triphenylphosphine)palladium(0) (0.511 g, 0.442 mmol) in DMF (22 mL) was degassed with nitrogen for 10 min and then heated at 120° C. overnight (ca. 20 h). After cooling, ethyl acetate (ca. 60 mL) and saturated aqueous sodium bicarbonate solution (ca. 20 mL) were added, the layers separated and the aqueous re-extracted with ethyl acetate (2×20 mL). The organic layers were combined, reduced in vacuo and then purified by chromatography on silica gel, eluting with a gradient of 0-40% ethyl acetate in cyclohexane to afford the title compound as a yellow solid (886 mg). LCMS (A) m/z: 272 [M+1]⁺, Rt 1.21 min (acidic).

### Description 49: Ethyl 4-(5-cyano-4-methyl-1H-indazol-1-yl)butanoate

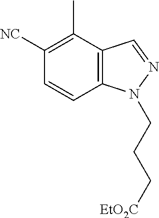

A mixture of ethyl 4-(5-bromo-4-methyl-1H-indazol-1-yl)butanoate (840 mg, 2.58 mmol) (Description 42), zinc cyanide (303 mg, 2.58 mmol) and tetrakis(triphenylphosphine)palladium(0) (298 mg, 0.258 mmol) in DMF (20 mL) was degassed with nitrogen for 10 min and the mixture then heated at 120° C. for 15 h. After cooling to RT, the reaction mixture was poured into saturated aqueous sodium bicarbonate solution (100 mL) and extracted with ethyl acetate (3×20 mL). The organic layers were combined, washed with brine (100 mL), dried over magnesium sulfate, filtered and reduced in vacuo. The residue was purified by chromatography on silica gel eluting with a gradient of 0-40% ethyl acetate in isohexane to give the title compound as a colourless oil (647 mg). LCMS (A) m/z: 272 [M+1]⁺, Rt 1.12 min (basic).

### Description 50: Ethyl 4-(6-chloro-5-cyano-1H-indazol-1-yl)butanoate

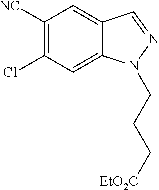

A mixture of ethyl 4-(5-bromo-6-chloro-1H-indazol-1-yl)butanoate (1.1979 g, 3.47 mmol) (Description 43), zinc cyanide (0.203 g, 1.733 mmol) and tetrakis(triphenylphosphine)palladium(0) (0.401 g, 0.347 mmol) in DMF (25 mL) was degassed with nitrogen for 10 min and the mixture then heated at at 120° C. overnight. The solution was cooled and ethyl acetate (ca. 60 mL) and saturated aqueous sodium bicarbonate solution (ca. 20 mL) added. The aqueous layer was separated and further extracted with ethyl acetate (2×20 mL) and the organics combined and reduced. The residue was purified by chromatography on silica gel eluting with a gradient of 0-30% ethyl acetate in isohexane to afford the title compound as an orange solid (835 mg). LCMS (A) m/z: 292 [M+1]⁺, Rt 1.20 min (acidic).

### Description 51: Ethyl 4-[5-cyano-6-(methyloxy)-1H-indazol-1-yl]butanoate

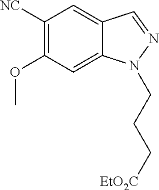

To 4-amino-5-methyl-2-(methyloxy)benzonitrile (931 mg, 5.74 mmol) (Description 24) in chloroform (20 mL) was added sequentially acetic anhydride (1.083 mL, 11.48 mmol), potassium acetate (676 mg, 6.89 mmol) a solution of 18-crown-6 (303 mg, 1.148 mmol) in chloroform (5 mL), and 1-butyl nitrite (1.432 mL, 12.05 mmol) and the mixture refluxed at 75° C. for 20 h. The dark brown reaction mixture was cooled then stirred with saturated aqueous sodium bicarbonate solution (25 mL) for 5 min. The aqueous was separated and extracted with DCM (2×20 mL) and the organics combined and reduced to afford a dark brown residue. This material was dissolved in methanol (25 mL) then treated with concentrated hydrochloric acid (1.178 mL, 14.35 mmol). The mixture was stirred at 80° C. for 3 h and then reduced to dryness. The residue was diluted with DMF (10 mL), and added potassium carbonate (3570 mg, 25.8 mmol) then ethyl 4-bromobutanoate (1.729 mL, 11.48 mmol). The resulting mixture was heated at 80° C. for 1 h then cooled and filtered. The filtrate was reduced and then purified by chromatography on silica gel, eluting with a gradient of 0-60% ethyl acetate in cyclohexane followed by MDAP (acidic conditions) to afford the title compound as a yellow oil (51 mg). LCMS (A) m/z: 288 [M+1]⁺, Rt 1.34 min (acidic). In addition, ethyl 4-[5-cyano-6-(methyloxy)-2H-indazol-2-yl]butanoate (42 mg) was isolated as a yellow solid.

### Description 52: Ethyl 3-oxetanylideneacetate

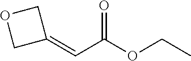

Carboethoxymethylene triphenylphosphorane (1.170 g, 3.36 mmol) was added to a solution of oxetan-3-one (0.22 g, 3.05 mmol) in DCM (6 mL) at 0° C. and the reaction allowed to warm to RT under nitrogen. After 20 min, reaction mixture was purified by chromatography on silica gel, eluting with 33% ethyl acetate in cyclohexane. The product containing fractions were reduced, re-dissolved in ethyl acetate and re-purified as above to afford the title compound as a colourless oil (434 mg) which was stored in the fridge. LCMS (B) m/z: 143 [M+1]⁺, Rt 0.67 min.

### Description 53: N-Hydroxy-1H-indazole-5-carboximidamide

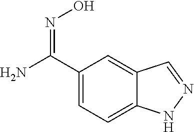

To a solution of 1H-indazole-5-carbonitrile (240 mg, 1.677 mmol) (Description 29) in ethanol (5 mL) was added hydroxylamine hydrochloride (233 mg, 3.35 mmol) and sodium bicarbonate (704 mg, 8.38 mmol) and the reaction mixture heated at 40° C. for 16 h. Further hydroxylamine hydrochloride (233 mg, 3.35 mmol) and sodium bicarbonate (704 mg, 8.38 mmol) were then added before heating at 90° C. for 16 h. The mixture was then cooled to RT and reduced in vacuo. The resulting solid was taken-up in ethyl acetate (30 mL), washed with water (2×20 mL), dried (phase separation cartridge) and reduced in vacuo to afford the title compound as an off-white solid (240 mg). LCMS (A) m/z: 177 [M+1]⁺, Rt 0.24 min (acidic).

### Description 54: N-Hydroxy-1H-indazole-4-carboximidamide

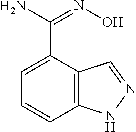

A solution of 1H-indazole-4-carbonitrile (837 mg, 5.85 mmol) in methanol (40 mL) was added to hydroxylamine hydrochloride (1.625 g, 23.38 mmol) and sodium bicarbonate (2.456 g, 29.2 mmol). The reaction mixture was then evacuated, purged with nitrogen and heated at 65° C. for the weekend. The reaction mixture was allowed to cool to RT, filtered and washed with methanol and the filtrate reduced to give an off white solid. Careful trituration with DCM was followed by filtration and evaporation of the colourless filtrate to give a cream solid. The solid and filtrate were therefore recombined and used directly in the next reaction. ¹H NMR (CD₃OD, 400 MHz) δ ppm: 7.37-7.43 (2H, m), 7.54 7.61 (1H, m), 8.34 (1H, d, J=1.0 Hz)

### Alternative:

1-Acetyl-1H-indazole-4-carbonitrile (11 g, 59 mmol) (Description 30) was added to a mixture of hydroxylamine hydrochloride (12.37 g, 178 mmol) and sodium carbonate (31.48 g, 297 mmol) in ethanol (120 mL) and the mixture refluxed for 6 h. The mixture was then concentrated in vacuo, dissolved in ethyl acetate and washed with water (×2). Both the water and ethyl acetate layers were concentrated in vacuo to afford the title compound as a yellow solid.

### Description 55: 1,1-Dimethylethyl 4-[(hydroxyamino)(imino)methyl]-1H-indazole-1-carboxylate

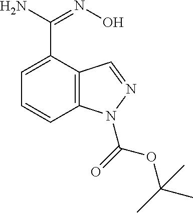

A mixture of 1,1-dimethylethyl 4-cyano-1H-indazole-1-carboxylate (0.190 g, 0.781 mmol) (Description 46), hydroxylamine hydrochloride (0.109 g, 1.562 mmol) and sodium bicarbonate (0.328 g, 3.91 mmol) in ethanol (5 mL) was stirred and heated at 50° C. for 3 h then left to cool overnight. The mixture was then diluted with ethyl acetate/water (40 mL each) and the organic layer separated, dried over magnesium sulfate and reduced to afford the title compound as a white solid (151 mg). LCMS (C) m/z: 277 [M+1]⁺, Rt 0.67 min.

### Description 56: Ethyl 3-{4-[(hydroxyamino)(imino)methyl]-1H-indazol-1-yl}propanoate

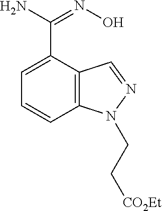

A mixture of ethyl 3-(4-cyano-1H-indazol-1-yl)propanoate (20.47 g, 84 mmol) (Description 45) and hydroxylamine hydrochloride (71.3 g, 673 mmol) in ethanol (700 mL), was treated portion-wise with sodium bicarbonate (70.7 g, 841 mmol). After stirring at RT for 10 min, the mixture was heated slowly to reflux for 2 h. The mixture was then allowed to cool slowly to RT and was then filtered and the filtrate concentrated. The resulting residue was triturated and sonicated with water (ca. 100 mL), filtered, washed with more water then dried at 50° C. in vacuo to afford the title compound (20.42 g). LCMS (B) m/z: 277 [M+1]⁺, Rt 0.55 min.

### Description 57: Ethyl 3-{5-[(hydroxyamino)(imino)methyl]-1H-indazol-1-yl}propanoate

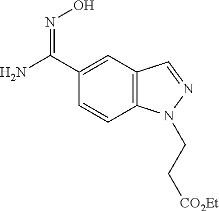

Ethyl 3-(5-cyano-1H-indazol-1-yl)propanoate (589 mg, 2.421 mmol) (Description 47) was dissolved in ethanol (10 mL) then, hydroxylamine hydrochloride (337 mg, 4.84 mmol) and sodium bicarbonate (1017 mg, 12.11 mmol) were added and the reaction mixture heated at 60° C. for 5 h and then overnight. The mixture was then cooled to RT and filtered and the filtrate reduced in vacuo. The residue was dissolved in ethyl acetate (ca. 30 mL) and then washed with water (ca. 20 mL). The aqueous phase was re-extracted with ethyl acetate (3×20 mL) and the organic layers combined, dried over magnesium sulfate, filtered and reduced in vacuo to give white solid (672 mg). LCMS (A) m/z: 277 [M+1]⁺, Rt 0.60 min (acidic).

### Description 58: Ethyl 4-{5-[(hydroxyamino)(imino)methyl]-6-methyl-1H-indazol-1-yl}butanoate

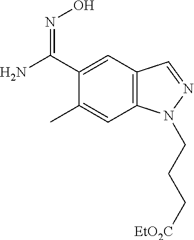

A mixture containing ethyl 4-(5-cyano-6-methyl-1H-indazol-1-yl)butanoate (342 mg, 1.261 mmol) (Description 48), sodium bicarbonate (529 mg, 6.30 mmol) and hydroxylamine hydrochloride (175 mg, 2.52 mmol) in ethanol (6 mL) was heated at 60° C. for 18 h. Additional hydroxylamine hydrochloride (350 mg) and sodium bicarbonate (318 mg) was added and heating continued at 80° C. for 3 h, then 100° C. for 20.5 h. The reaction mixture was cooled and filtered and the filtrate reduced. The residue was then taken up in ethyl acetate (30 mL) and water (10 mL) and the aqueous separated and re-extracted with ethyl acetate (3×10 mL). The organic layers were then combined, dried over magnesium sulfate and reduced to afford the title compound as a yellow gum (118 mg). LCMS (A) m/z: 305 [M+1]⁺, Rt 0.68 min (acidic).

### Description 59: Ethyl 4-{5[(hydroxyamino)(imino)methyl]-4-methyl-1H-indazol-1-yl}butanoate

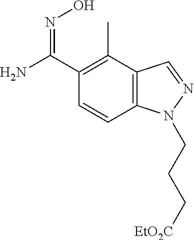

A mixture containing ethyl 4-(5-cyano-4-methyl-1H-indazol-1-yl)butanoate (640 mg, 2.359 mmol) (Description 49), sodium bicarbonate (1585 mg, 18.87 mmol) and hydroxylamine hydrochloride (656 mg, 9.44 mmol) in ethanol (15 mL) was heated at 85° C. for 18 h. The reaction mixture was then cooled and filtered and the filtrate reduced. The residue was then taken up in ethyl acetate (30 mL) and water (10 mL) and the aqueous separated and re-extracted with ethyl acetate (3×10 mL). The combined organic layers were then reduced to afford the title compound as a yellow gum (209 mg). LCMS (A) m/z: 305 [M+1]⁺, Rt 0.68 min (acidic).

### Description 60: Ethyl 4-{5-[(hydroxyamino)(imino)methyl]-1H-indazol-1-yl}butanoate

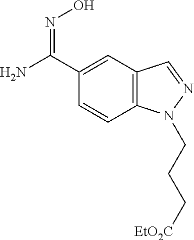

A solution of ethyl 4-(5-cyano-1H-indazol-1-yl)butanoate (250 mg, 0.972 mmol) (Description 44) in ethanol (4 mL) was treated with hydroxylamine hydrochloride (135 mg, 1.943 mmol) and sodium bicarbonate (408 mg, 4.86 mmol) and the reaction mixture stirred at 60° C. under nitrogen over the weekend. The mixture was then cooled to RT and filtered and the resulting filtrate concentrated in vacuo. The resulting oil was re-dissolved in ethyl acetate and washed with water, and then the aqueous phase extracted with ethyl acetate (×3). The organic layers were combined, dried (phase separator) and then reduced in vacuo to afford the title compound as an oil which was dried under high vacuum (276 mg). LCMS (A) m/z: 291 [M+1]⁺, Rt 0.77 min (basic).

### Description 61: N-Hydroxy-6-methyl-1H-indazole-5-carboximidamide

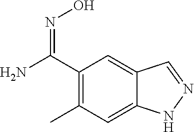

A mixture containing 6-methyl-1H-indazole-5-carbonitrile (151 mg, 0.961 mmol) (Description 35), sodium bicarbonate (484 mg, 5.76 mmol) and hydroxylamine hydrochloride (247 mg, 3.55 mmol) in ethanol (8 mL) was heated at 65° C. for 65 h. Additional hydroxylamine hydrochloride (247 mg) and sodium bicarbonate (500 mg) were then added and heating continued at 100° C. for 8 h. Further hydroxylamine hydrochloride (140 mg) and sodium bicarbonate (200 mg) were added and heating continued at 90° C. for 16 h. The reaction mixture was then cooled and filtered and the filtrate reduced. The residue was then taken up in ethyl acetate (30 mL) and water (10 mL) and the aqueous layer separated and re-extracted with ethyl acetate (3×10 mL). The organic layers were combined and then dried over magnesium sulfate and then reduced to afford the title compound as a pale white gum (182 mg). LCMS (A) m/z: 191 [M+1]⁺, Rt 0.52 min (basic).

### Description 62: 6-Fluoro-N-hydroxy-1H-indazole-5-carboximidamide

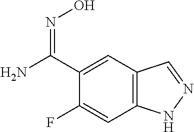

A mixture containing 6-fluoro-1H-indazole-5-carbonitrile (464 mg, 2.88 mmol) (Description 27), sodium bicarbonate (2903 mg, 34.6 mmol) and hydroxylamine hydrochloride (1001 mg, 14.40 mmol) in ethanol (20 mL) was heated at 90° C. for 4 h and then stirred at RT for 16 h. The reaction mixture was then filtered and the filtrate reduced and dried to afford the title compound as a yellow solid (642 mg). LCMS (A) m/z: 195 [M+1]⁺, Rt 0.50 min (basic).

### Description 63: 6-Chloro-N-hydroxy-1H-indazole-5-carboximidamide

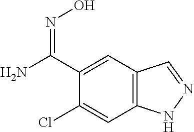

A mixture containing 6-chloro-1H-indazole-5-carbonitrile (579 mg, 3.26 mmol) (Description 38), sodium bicarbonate (2739 mg, 32.6 mmol) and hydroxylamine hydrochloride (906 mg, 13.04 mmol) in ethanol (20 mL) was heated at 85° C. for ca. 65 h. The mixture was then filtered and the filtrate reduced and dried to afford the title compound as a light brown sticky solid (666 mg). LCMS (A) m/z: 211 [M+1]⁺, Rt 0.53 min (basic).

### Description 64: 6-Ethyl-N-hydroxy-1H-indazole-5-carboximidamide

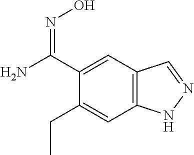

A mixture containing 6-ethyl-1H-indazole-5-carbonitrile (240 mg, 1.402 mmol) (Description 21), sodium bicarbonate (1649 mg, 19.63 mmol) and hydroxylamine hydrochloride (585 mg, 8.41 mmol) in ethanol (15 mL) was heated at 85° C. overnight and then over the weekend. Additional hydroxylamine hydrochloride (585 mg, 8.41 mmol) and sodium bicarbonate (1649 mg, 19.63 mmol) were then added and heating continued at 85° C. for a further night. The reaction mixture was then concentrated to near dryness, and the residue diluted with ethyl acetate (30 mL), washed with brine (15 mL), reduced and dried to afford the title compound as a light brown sticky solid (277 mg). LCMS (A) m/z: 205 [M+1]⁺, Rt 0.65 min (basic).

### Description 65: N-Hydroxy-6-(methyloxy)-1H-indazole-5-carboximidamide

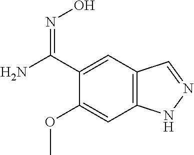

A mixture containing 6-(methyloxy)-1H-indazole-5-carbonitrile (116 mg, 0.670 mmol) (Description 25), sodium bicarbonate (1407 mg, 16.75 mmol), and hydroxylamine hydrochloride (465 mg, 6.70 mmol) in ethanol (15 mL) was heated at 90° C. over ca. 55 h. The reaction mixture was filtered and the filtrate reduced to dryness. The residue diluted with DCM (30 mL) and washed with water (10 mL). The aqueous was separated and extracted with 10% methanol in DCM (3×20 mL). The organics and aqueous were then combined and reduced to afford the title compound as a reddish brown solid (170 mg) which was used directly without further purification. LCMS (A) m/z: 207 [M+1]⁺, Rt 0.53 min (basic)

### Description 66: Ethyl 4-{6-chloro-5-[(hydroxyamino)(imino)methyl]-1H-indazol-1-yl}butanoate

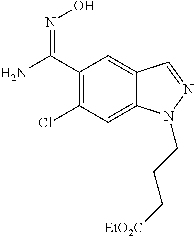

A suspension of ethyl 4-(6-chloro-5-cyano-1H-indazol-1-yl)butanoate (835 mg, 2.86 mmol) (Description 50), hydroxylamine hydrochloride (994 mg, 14.31 mmol) and sodium bicarbonate (2645 mg, 31.5 mmol) in ethanol (25 mL) was heated at 80° C. overnight. The suspension was cooled and filtered and the filtrate reduced to afford the title compound as a yellow gum (624 mg) which was used directly without further purification. LCMS (A) m/z: 325 [M+1]⁺, Rt 0.74 min (acidic).

### Description 67: Ethyl 4-{6-ethyl-5-[(hydroxyamino)(imino)methyl]-1H-indazol-1-yl}butanoate

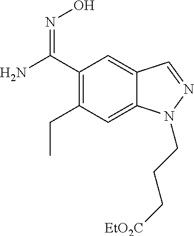

A suspension of ethyl 4-(5-cyano-6-ethyl-1H-indazol-1-yl)butanoate (471 mg, 1.651 mmol) (Description 39), hydroxylamine hydrochloride (574 mg, 8.25 mmol) and sodium bicarbonate (1525 mg, 18.16 mmol) in ethanol (30 mL) was heated at 80° C. over the weekend. The mixture was cooled, the solid filtered and the filtrate reduced to afford the title compound as a yellow gum (526 mg) which was used directly without further purification. LCMS (A) m/z: 319 [M+1]⁺, Rt 0.76 min (acidic).

### Description 68: Ethyl 4-[5-[(hydroxyamino)(imino)methyl]-6-(methyloxy)-1H-indazol-1-yl]butanoate

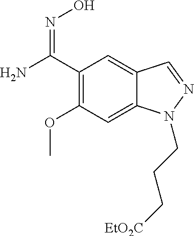

A mixture of ethyl 4-[5-cyano-6-(methyloxy)-1H-indazol-1-yl]butanoate (51 mg, 0.178 mmol) (Description 51), hydroxylamine hydrochloride (126 mg, 1.813 mmol) and sodium bicarbonate (660 mg, 7.86 mmol) in ethanol (8 mL) was stirred at 80° C. overnight. The mixture was cooled and additional hydroxylamine hydrochloride (247 mg, 3.55 mmol) and sodium bicarbonate (660 mg, 7.86 mmol) were added and the mixture heated at 85° C. for 7 h. After cooling the mixture was filtered and the filtrate reduced to afford the title compound as a colourless gum (68 mg) which was used directly without further purification. LCMS (A) m/z: 321 [M+1]⁺, Rt 0.80 min (acidic).

### Description 69: Methyl 3-[4-(5-{3-chloro-4-[(1-methylethyl)oxy]phenyl}-1,2,4-oxadiazol-3-yl)-1H-indazol-1-yl]-2-methylpropanoate

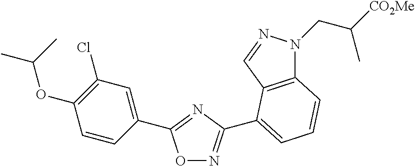

A mixture of 4-(5-{3-Chloro-4-[(1-methylethyl)oxy]phenyl}-1,2,4-oxadiazol-3-yl)-1H-indazole (Example 4) (54 mg, 0.152 mmol), methyl 3-bromo-2-methylpropanoate (30.3 mg, 0.167 mmol), and cesium carbonate (94 mg, 0.289 mmol) in DMF (2 mL) was purged with nitrogen and heated at 80° C. overnight under nitrogen. The mixture was allowed to cool to RT and stand for 4 h. The reaction was then diluted with DCM, reduced and the residue partitioned between DCM and brine. The organic phase was passed through a phase separation cartridge and reduced. The residue was purified by chromatography on silica gel eluting with 7-60% ethyl acetate in cyclohexane and the desired fractions combined and reduced to give the title compound as a white solid (70 mg). LCMS (B) m/z: 455 [M+1]⁺, Rt 1.53 min.

### Description 70: Methyl 5-[4-(5-{3-chloro-4-[(1-methylethyl)oxy]phenyl}-1,2,4-oxadiazol-3-yl)-1H-indazol-1-yl]pentanoate

### Description 71: Methyl 5-[4-(5-{3-chloro-4-[(1-methylethyl)oxy]phenyl}-1,2,4-oxadiazol-3-yl)-2H-indazol-2-yl]pentanoate

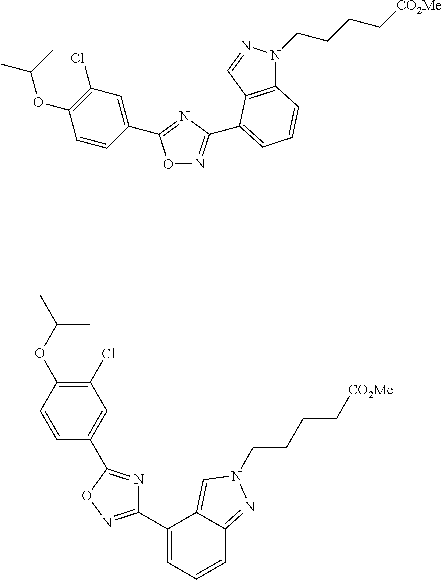

4-(5-{3-Chloro-4-[(1-methylethyl)oxy]phenyl}-1,2,4-oxadiazol-3-yl)-1H-indazole (115 mg, 0.324 mmol) (Example 4), potassium carbonate (134 mg, 0.972 mmol) and methyl 5-bromovalerate (126 mg, 0.648 mmol) were heated at 70° C. under nitrogen, overnight in DMF (3 mL). The mixture was allowed to cool to RT, was diluted with ethyl acetate (5 mL) and washed with saturated aqueous sodium bicarbonate solution. The aqueous phase was extracted into more ethyl acetate and the combined organics washed with brine, dried over sodium sulfate and reduced to give a pale yellow oil, which solidified to a yellow solid on standing. Purification by chromatography on silica gel, eluting with 7-60% ethyl acetate in cyclohexane afforded methyl 5-[4-(5-{3-chloro-4-[(1-methylethyl)oxy]phenyl}-1,2,4-oxadiazol-3-yl)-1H-indazol-1-yl]pentanoate as a white solid (54 mg). LCMS (B) m/z: 469 [M+1]⁺, Rt 1.55 min and methyl 5-[4-(5-{3-chloro-4-[(1-methylethyl)oxy]phenyl}-1,2,4-oxadiazol-3-yl)-2H-indazol-2-yl]pentanoate as a colourless oil (54 mg). LCMS (B) m/z: 469 [M+1]⁺, Rt 1.51 min.

### Description 72: Ethyl {3-[4-(5-{3-chloro-4[(1-methylethyl)oxy]phenyl}-1,2,4-oxadiazol-3-yl)-1H-indazol-1-yl]-3-oxetanyl}acetate

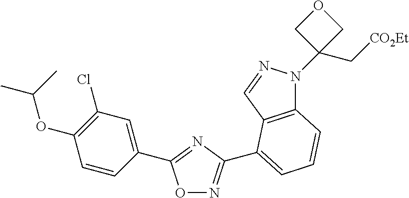

DBU (0.019 mL, 0.127 mmol) was added to a suspension of 4-(5-{(3-chloro-4-[(1-methylethyl)oxy]phenyl}-1,2,4-oxadiazol-3-yl)-1H-indazole (45 mg, 0.127 mmol) (Example 4) and ethyl 3-oxetanylideneacetate (50 mg, 0.352 mmol) (Description 52) in acetonitrile (2 mL) and the pale yellow reaction mixture heated at 70° C. under argon overnight. The mixture was then cooled to RT, and then re-heated at 80° C. for 7 h. More acetonitrile (1 was added and reaction mixture heated for another night. The mixture was then allowed to cool to RT, ethyl acetate (5 mL) was added and the mixture washed with saturated aqueous sodium bicarbonate solution. The aqueous was extracted into more ethyl acetate and the combined organics washed with brine, dried over sodium sulfate and reduced. Purification by chromatography on silica gel, eluting with 7-60% ethyl acetate in cyclohexane afforded the title compound as a colourless residue (30 mg). ¹H NMR (CDCl₃, 400 MHz) δ ppm: 1.06 (3H, t, J=7.0 Hz), 1.46 (6H, d, J=6.0 Hz), 3.40 (2H, s), 3.98 (2H, q, J=7.0 Hz), 4.73 (1H, spt, J=6.0 Hz), 5.13 (2H, d, J=7.0 Hz), 5.46 (2H, d, J=6.5 Hz), 7.08 (1H, d, J=8.5 Hz), 7.53 (1H, m), 7.68 (1H, d, J=8.0 Hz), 8.07-8.13 (2H, m), 8.28 (1H, d, J=2.0 Hz), 8.72 (1H, s).

### Description 73: Ethyl 3-[4-(5-{3-chloro-4-[(1-methylethyl)oxy]phenyl}-1,2,4-oxadiazol-3-yl)-1H-indazol-1-yl]propanoate

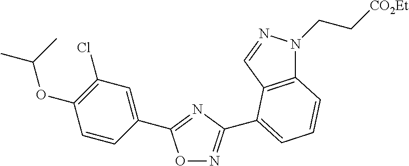

Ethyl 3-{4-[(hydroxyamino)(imino)methyl]-1H-indazol-1-yl}propanoate (3 g, 10.86 mmol) (Description 56) in dry DMF (20 mL), cooled in ice bath, was treated with triethylamine (1.816 mL, 13.03 mmol), followed by the drop-wise addition of a solution of 3-chloro-4-[(1-methylethyl)oxy]benzoyl chloride (2.66 g, 11.40 mmol) (Description 11) in dry DMF (10 mL). The reaction mixture was stirred at RT for 1 h, then 120° C. for 3 h, followed by RT for ca. 36 h. The mixture was filtered, washed with ethyl acetate and the brown filtrate concentrated in vacuo. The residue was re-dissolved in ethyl acetate (50 mL) and treated with 20% aqueous sodium carbonate (3×50 mL) and the solution vigorously stirred for 15 min each time. The organic phase was dried and concentrated to a pale brown crystalline solid which was stirred at RT with a solution of 10% aqueous sodium carbonate for 4 h. The solid was filtered, washed with water and dried, in vacuo at 50° C., to give a pale straw coloured solid which was purified by chromatography on silica gel eluting with a gradient of ethyl acetate in cyclohexane to afford the title compound as a colourless crystalline solid (3.644 g). LCMS (B) m/z: 455 [M+1]⁺, Rt 1.55 min.

### Description 74: Ethyl 3-[4-(5-{3-chloro-4-[(1-methylethyl)oxy]phenyl}-1,2,4-oxadiazol-3-yl)-1H-indazol-1-yl]-2-hydroxypropanoate

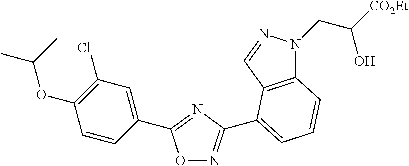

TMEDA (0.214 mL, 1.418 mmol) and LDA (1.8M; 0.315 mL, 0.567 mmol) were added to a solution of ethyl 3-[4-(5-{3-chloro-4-[(1-methylethyl)oxy]phenyl}-1,2,4-oxadiazol-3-yl)-1H-indazol-1-yl]propanoate (129 mg, 0.284 mmol) (Description 73) in THF (10 mL) at −78° C. under nitrogen. After 30 min at −78° C., (1S)-(+)-(10)-(camphorylsulfonyl)oxaziridine (98 mg, 0.425 mmol) in THF (1 mL) was added and the reaction mixture was warmed to −40° C. and stirred for 1.5 h. The reaction was then quenched with saturated ammonium chloride and allowed to stand overnight with warming to RT. The mixture was then extracted into ethyl acetate (2×20 mL), the combined organics were washed with brine, dried over sodium sulfate and reduced to give a pale yellow solid. This crude material was purified by MDAP (acidic conditions) to afford the title compound as a white solid (50 mg). LCMS (B) m/z: 471 [M+1]⁺, Rt 1.43 min. The stereochemistry of this compound was not determined.

### Description 75: Ethyl 3-[5-(5-{5-chloro-6-[(1-methylethyl)oxy]-3-pyridinyl}-1,2,4-oxadiazol-3-yl)-1H-indazol-1-yl]propanoate

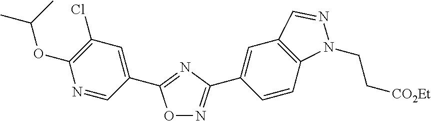

To a solution of 5-chloro-6-[(1-methylethyl)oxy]-3-pyridinecarboxylic acid (164 mg, 0.760

mmol) (Description 13) in DMF (4 mL) was added HATU (330 mg, 0.869 mmol) and DIPEA (0.152 mL, 0.869 mmol) and the mixture was stirred at RT for 10 min. Ethyl 3-{5-[(hydroxyamino)(imino)methyl]-1H-indazol-1-yl}propanoate (200 mg, 0.724 mmol) (Description 57) was then added and the mixture was stirred at RT for 1 h, followed by 80° C. overnight. The mixture was then cooled to RT and reduced in vacuo, and the residue re-dissolved in ethyl acetate (ca. 30 mL) and washed with water (ca. 10 mL). The organic phase was dried over magnesium sulfate, filtered and concentrated. The crude residue was purified by chromatography on silica gel eluting with a gradient of 0-50% ethyl acetate in isohexane to afford the title compound as a white solid (208 mg). LCMS (A) m/z: 456 [M+1]⁺, Rt 1.61 min (acidic).

### Description 76: Ethyl 4-[5-(5-{5-chloro-6-[(1-methylethyl)oxy]-3-pyridinyl}-1,2,4-oxadiazol-3-yl)-6-methyl-1H-indazol-1-yl]butanoate

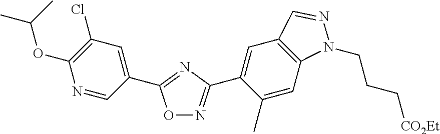

A solution of 5-chloro-6-[(1-methylethyl)oxy]-3-pyridinecarboxylic acid (107 mg, 0.496 mmol), (Description 13) HATU (177 mg, 0.465 mmol) and DIPEA (0.081 mL, 0.465 mmol) in DMF (1 mL) was stirred at RT for 15 min. A solution of ethyl 4-{5-[(hydroxyamino)(imino)methyl]-6-methyl-1H-indazol-1-yl}butanoate (118 mg, 0.388 mmol) (Description 58) in DMF (2 mL) was then added drop-wise and the mixture heated at 80° C. for 16 h. The mixture was then reduced in vacuo and the residue purified by chromatography on silica gel eluting with a gradient of 0-30% ethyl acetate in isohexane to afford the title compound as a white solid (66 mg). LCMS (A) m/z: 484 [M+1]⁺, Rt 1.66 min (acidic).

### Description 77: Ethyl 4-[5-(5-{5-chloro-6-[(1-methylethyl)oxy]-3-pyridinyl}-1,2,4-oxadiazol-3-yl)-4-methyl-1H-indazol-1-yl]butanoate

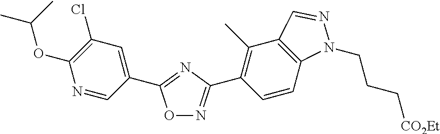

A solution of 5-chloro-6-[(1-methylethyl)oxy]-3-pyridinecarboxylic acid (178 mg, 0.824 mmol) (Description 13), PyBOP (429 mg, 0.824 mmol), and DIPEA (0.144 mL, 0.824 mmol) in DMF (2 mL) was stirred at RT for 15 min. A solution of ethyl 4-{5-[(hydroxyamino)(imino)methyl]-4-methyl-1H-indazol-1-yl}butanoate (209 mg, 0.687 mmol) (Description 59) in DMF (3 mL) was added drop-wise and the mixture heated at 80° C. for 16 h. The mixture was then reduced in vacuo and the residue purified by chromatography on silica gel, eluting with a gradient of 0-30% ethyl acetate in isohexane to afford the title compound as an off-white solid (91 mg). LCMS (A) m/z: 484 [M+1]⁺, Rt 1.65 min (acidic).

### Description 78: Ethyl 4-[5-(5-{5-cyano-6-[(1-methylethyl)oxy]-3-pyridinyl}-1,2,4-oxadiazol-3-yl)-1H-indazol-1-yl]butanoate

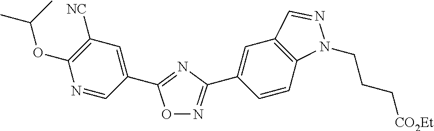

To a solution of 5-cyano-6-[(1-methylethyl)oxy]-3-pyridinecarboxylic acid (107 mg, 0.519 mmol) (Description 17) in DMF (1.5 mL) was added HATU (179 mg, 0.471 mmol) and DIPEA (0.109 mL, 0.623 mmol) and the mixture stirred under nitrogen at RT for 20 min. A solution of ethyl 4-{(5-[(hydroxyamino)(imino)methyl]-1H-indazol-1-yl}butanoate (128 mg, 0.441 mmol) (Description 60) in DMF (1.5 mL) was then added and the mixture left to stir for 1 h under nitrogen at RT, before being heated to 80° C. and left to reflux overnight. The reaction mixture was cooled to RT, reduced in vacuo and the resulting brown oil re-dissolved in ethyl acetate, and washed with water. The aqueous phase was then extracted with ethyl acetate (×3) and the organic layers combined, dried using a phase separator and the solvent removed in vacuo. Purification by chromatography on silica gel eluting with a gradient of 0-35% ethyl acetate in isohexane afforded a cream solid which was dried under high vacuum to afford the title compound (78 mg). LCMS (A) m/z: 461 [M+1], Rt 1.45 min (acidic).

### Description 79: Ethyl 445-(5-{5-chloro-6-[(1-methylethyl)oxy]-3-pyridinyl}-1,2,4-oxadiazol-3-yl)-6-fluoro-1H-indazol-1-yl]butanoate

### Description 80: Ethyl 4-[5-(5-{5-chloro-6-[(1-methylethyl)oxy]-3-pyridinyl}-1,2,4-oxadiazol-3-yl)-6-fluoro-2H-indazol-2-yl]butanoate

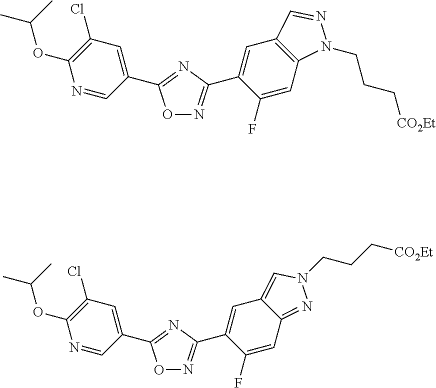

A mixture of 5-(5-{5-chloro-6-[(1-methylethyl)oxy]-3-pyridinyl}-1,2,4-oxadiazol-3-yl)-6-fluoro-1H-indazole (165 mg, 0.441 mmol) (Example 16), cesium carbonate (173 mg, 0.530 mmol) and ethyl 4-bromobutanoate (0.081 mL, 0.530 mmol) in DMF (5 mL) was heated at 80° C. for 1 h. The mixture was reduced to near dryness and the residue purified by chromatography on silica gel eluting with a gradient of 0-100% ethyl acetate in isohexane to afford ethyl 4-[5-(5-{5-chloro-6-[(1-methylethyl)oxy]-3-pyridinyl}-1,2,4-oxadiazol-3-yl)-6-fluoro-1H-indazol-1-yl]butanoate as a white solid (117 mg). LCMS (A) m/z: 488 [M+1]⁺, Rt 1.62 min (acidic) and ethyl 4-[5-(5-{5-chloro-6-[(1-methylethyl)oxy]-3-pyridinyl}-1,2,4-oxadiazol-3-yl)-6-fluoro-2H-indazol-2-yl]butanoate as a white solid (72 mg). LCMS (A) m/z: 488 [M+1]⁺, Rt 1.55 min (acidic).

### Description 81: Ethyl 4-[6-chloro-5-(5-{5-chloro-6-[(1-methylethyl)oxy]-3-pyridinyl}-1,2,4-oxadiazol-3-yl)-1H-indazol-1-yl]butanoate

### Description 82: Ethyl 4-[6-chloro-5-(5-{5-chloro-6-[(1-methylethyl)oxy]-3-pyridinyl}-1,2,4-oxadiazol-3-yl)-2H-indazol-2-yl]butanoate

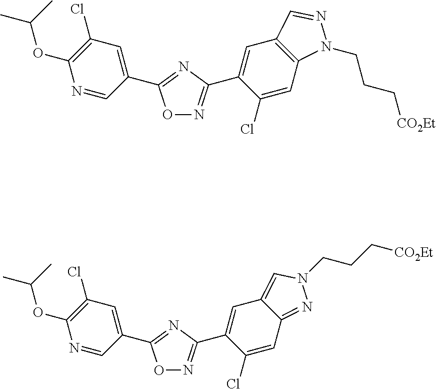

A mixture of 6-chloro-5-(5-{5-chloro-6-[(1-methylethyl)oxy]-3-pyridinyl}-1,2,4-oxadiazol-3-yl)-1H-indazole (120 mg, 0.308 mmol) (Example 19), cesium carbonate (125 mg, 0.384 mmol) and ethyl 4-bromobutanoate (0.058 mL, 0.384 mmol) in DMF (3.5 mL) was heated at 80° C. for 1 h. The mixture was reduced to near dryness and the residue purified by chromatography on silica gel eluting with a gradient of 0-100% ethyl acetate in isohexane to afford ethyl 4-[6-chloro-5-(5-{5-chloro-6-[(1-methylethyl)oxy]-3-pyridinyl}-1,2,4-oxadiazol-3-yl)-1H-indazol-1-yl]butanoate as a white solid (90 mg). LCMS (A) m/z: 504 [M+1]⁺, Rt 1.65 min (acidic) and ethyl 4-[6-chloro-5-(5-{5-chloro-6-[(1-methylethyl)oxy]-3-pyridinyl}-1,2,4-oxadiazol-3-yl)-2H-indazol-2-yl]butanoate as a white solid (53 mg). LCMS (A) m/z: 504 [M+1]⁺, Rt 1.60 min (acidic).

### Description 83: Ethyl 4-[5-(5-{5-chloro-6-[(1-methylethyl)oxy]-3-pyridinyl}-1,2,4-oxadiazol-3-yl)-6-ethyl-1H-indazol-1-yl]butanoate

### Description 84: Ethyl 4-[5-(5-{5-chloro-6-[(1-methylethyl)oxy]-3-pyridinyl}-1,2,4-oxadiazol-3-yl)-6-ethyl-2H-indazol-2-yl]butanoate

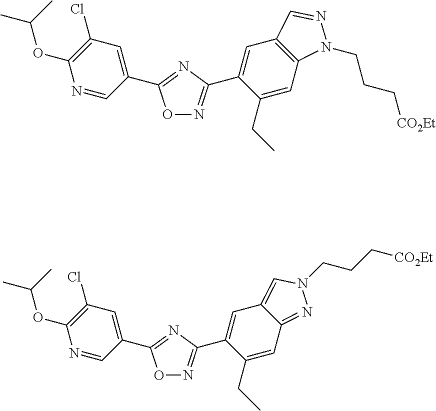

A mixture of 5-(5-{5-chloro-6-[(1-methylethyl)oxy]-3-pyridinyl}-1,2,4-oxadiazol-3-yl)-6-ethyl-1H-indazole (130 mg, 0.339 mmol) (Example 22), cesium carbonate (138 mg, 0.423 mmol) and ethyl 4-bromobutanoate (0.064 mL, 0.423 mmol) in DMF (4 mL) was heated at 80° C. for 3 h. The mixture was reduced to near dryness and the residue purified by chromatography on silica gel eluting with a gradient of 0-30% ethyl acetate in isohexane to afford ethyl 4-[5-(5-{5-chloro-6-[(1-methylethyl)oxy]-3-pyridinyl}-1,2,4-oxadiazol-3-yl)-6-ethyl-1H-indazol-1-yl]butanoate as a yellow oil (136 mg). LCMS (A) m/z: 498 [M+1]⁺., Rt 1.69 min (acidic) and ethyl 4-[5-(5-{5-chloro-6-[(1-methylethyl)oxy]-3-pyridinyl}-1,2,4-oxadiazol-3-yl)-6-ethyl-2H-indazol-2-yl]butanoate as a white solid (58 mg). LCMS (A) m/z: 498 [M+1]⁺, Rt 1.63 min (acidic).

### Description 85: Ethyl 3-[5-(5-{5-cyano-6-[(1-methylethyl)oxy]-3-pyridinyl}-1,2,4-oxadiazol-3-yl)-1H-indazol-1-yl]propanoate

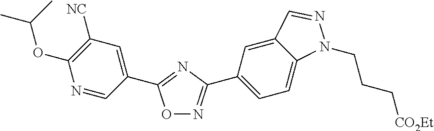

To a solution of ethyl 3-{5-[(hydroxyamino)(imino)methyl]-1H-indazol-1-yl}propanoate (201 mg, 0.727 mmol) (Description 57) in DMF (4 mL) was added 5-cyano-6-[(1-methylethyl)oxy]-3-pyridinecarboxylic acid (150 mg, 0.727 mmol) (Description 17), EDC (153 mg, 0.800 mmol) and the reaction mixture heated to 80° C. for 16 h. The mixture was then diluted with ethyl acetate (30 mL) and washed with water (30 mL). The aqueous phase was then extracted with ethyl acetate (3×30 mL) and the organics combined, dried over a phase separation cartridge and reduced in vacuo. Purification by chromatography on silica gel eluting with a gradient of 0-50% ethyl acetate in isohexane afforded the title compound as a white solid (190 mg). ¹H NMR (CDCl₃, 400 MHz) δ ppm: 1.19 (3H, m), 1.48 (6H, d, J=6.0 Hz), 3.03 (2H, t, J=7.0 Hz, 3 H), 4.11 (2H, q, J=7.0 Hz), 4.73 (2H, t, 7.0 Hz), 5.58 (1H, spt, J=6.0 Hz), 7.63 (1H, dt, J 9.0, 1.0 Hz), 8.14 (1H, d, J=1.0 Hz), 8.18 (1H, dd, J 9.0, 1.5 Hz), 8.59 (1H, dd, J 1.5, 1.0 Hz), 8.67 (1H, d, J=2.5 Hz), 9.17 (1H, d, J=2.5 Hz).

### Description 86: Ethyl 4-[6-chloro-5-(5-{5-cyano-6-[(1-methylethyl)oxy]-3-pyridinyl}-1,2,4-oxadiazol-3-yl)-1H-indazol-1-yl]butanoate

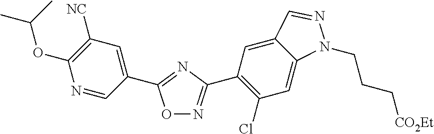

A mixture of 5-cyano-6-[(1-methylethyl)oxy]-3-pyridinecarboxylic acid (79 mg, 0.382 mmol) (Description 17), HATU (145 mg, 0.382 mmol) and DIPEA (0.067 mL, 0.382 mmol) in DMF (2.5 mL) was stirred at RT for 15 min, followed by drop-wise addition of ethyl 4-{6-chloro-5-[(hydroxyamino)(imino)methyl]-1H-indazol-1-yl}butanoate (164 mg, 0.318 mmol) (Description 66) in DMF (2.5 mL). The mixture was heated at 80° C. for ca. 18 h and was then reduced to dryness and the residue purified by chromatography on silica gel eluting with a gradient of 0-40% ethyl acetate in isohexane to afford the title compound as a white solid (129 mg). LCMS (A) m/z: 495 [M+1]⁺, Rt 1.51 min (acidic).

### Description 87: Ethyl 4-[6-chloro-5-(5-{3-cyano-4-[(1-methylethyl)oxy]phenyl}-1,2,4-oxadiazol-3-yl)-1H-indazol-1-yl]butanoate

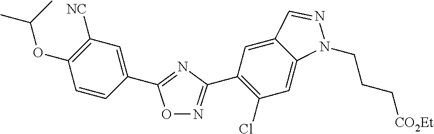

A mixture of 3-cyano-4-[(1-methylethyl)oxy]benzoic acid (192 mg, 0.933 mmol) (Description 7), HATU (355 mg, 0.933 mmol) and DIPEA (0.163 mL, 0.933 mmol) in DMF (4 mL) was stirred at RT for 10 min, followed by drop-wise addition of ethyl 4-{(6-chloro-5-[(hydroxyamino)(imino)methyl]-1H-indazol-1-yl}butanoate (401 mg, 0.778 mmol) (Description 66) in DMF (4 mL). The mixture was heated at 80° C. for ca. 18 h and was then reduced to dryness and the residue purified twice by chromatography on silica gel eluting with a gradient of 0-40% ethyl acetate in isohexane gradient 0-40% to afford the title compound as an off-white solid (243 mg). LCMS (A) m/z: 494 [M+1]⁺, Rt 1.55 min (acidic).

### Description 88: Ethyl 4-[5-(5-{3-cyano-4-[(1-methylethyl)oxy]phenyl}-1,2,4-oxadiazol-3-yl)-6-methyl-1H-indazol-1-yl]butanoate

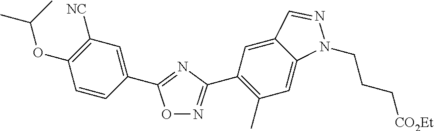

A mixture of 3-cyano-4-[(1-methylethyl)oxy]benzoic acid (196 mg, 0.957 mmol) (Description 7), HATU (364 mg, 0.957 mmol) and DIPEA (0.167 mL, 0.957 mmol) in DMF (4 mL) was stirred at RT for 10 min, followed by drop-wise addition of ethyl 4-{5-[(hydroxyamino)(imino)methyl]-6-methyl-1H-indazol-1-yl}butanoate (466.7 mg, 0.797 mmol) (Description 58) in DMF (4 mL). The mixture was heated at 80° C. for ca. 18 h and was then reduced to dryness and the residue purified by chromatography on silica gel eluting with a gradient of 0-50% ethyl acetate in isohexane to afford the title compound as a white solid (136 mg). LCMS (A) m/z: 474 [M+1]⁺, Rt 1.60 min (acidic).

### Description 89: Ethyl 4-[5-(5-{3-chloro-4-[(1-methylethyl)oxy]phenyl}-1,2,4-oxadiazol-3-yl)-6-methyl-1H-indazol-1-yl]butanoate

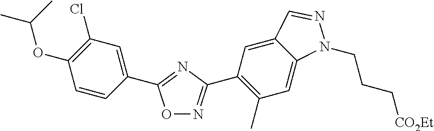

A mixture of 3-chloro-4-[(1-methylethyl)oxy]benzoic acid (187 mg, 0.870 mmol) (Description 10), HATU (388 mg, 1.020 mmol) and DIPEA (0.182 mL, 1.044 mmol) in DMF (4 mL) was stirred at RT for 10 min, followed by drop-wise addition of ethyl 4-{5-[(hydroxyamino)(imino)methyl]-6-methyl-1H-indazol-1-yl}butanoate (509 mg, 0.870 mmol) (Description 58) in DMF (4 mL). The mixture was heated at 80° C. for ca. 18 h and was then reduced to dryness and the residue purified by chromatography on silica gel eluting with a gradient of 0-40% ethyl acetate in isohexane to afford the title compound as a white solid (143 mg). LCMS (A) m/z: 483 [M+1]⁺, Rt 1.74 min (acidic).

### Description 90: Ethyl 4-[5-(5-{3-cyano-4-[(1-methylethyl)oxy]phenyl}-1,2,4-oxadiazol-3-yl)-6-ethyl-1H-indazol-1-yl]butanoate

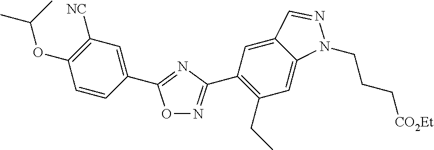

A mixture of 3-cyano-4-[(1-methylethyl)oxy]benzoic acid (82 mg, 0.400 mmol) (Description 7), HATU (225 mg, 0.593 mmol) and DIPEA (0.104 mL, 0.593 mmol) in DMF (4 mL) was stirred at RT for 10 min, followed by drop-wise addition of ethyl 4-{6-ethyl-5-[(hydroxyamino)(imino)methyl]-1H-indazol-1-yl}butanoate (526 mg, 0.363 mmol) (Description 67) in DMF (4 mL). The mixture was heated at 80° C. for ca. 18 h and was then reduced to dryness and the residue purified by chromatography on silica gel eluting with a gradient of 0-60% ethyl acetate in cyclohexane to afford the title compound as yellow oil (65 mg). LCMS Rt 1.54 min (acidic).

### Description 91: Ethyl 4-[5-(5-{3-cyano-4-[(1-methylethyl)oxy]phenyl}-1,2,4-oxadiazol-3-yl)-6-(methyloxy)-1H-indazol-1-yl]butanoate

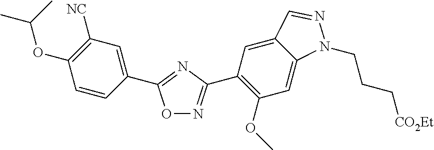

A mixture of 3-cyano-4-[(1-methylethyl)oxy]benzoic acid (40.5 mg, 0.197 mmol) (Description 7), HATU (75 mg, 0.197 mmol), DIPEA (0.034 mL, 0.197 mmol) in DMF (1 mL) was stirred at RT for 10 min, followed by drop-wise addition of ethyl 4-[5-[(hydroxyamino)(imino)methyl]-6-(methyloxy)-1H-indazol-1-yl]butanoate (68 mg, 0.132 mmol) (Description 68) in DMF (3 mL). The mixture was heated at 80° C. for ca. 18 h and was then reduced to dryness and the residue purified by chromatography on silica gel eluting with a gradient of 0-60% ethyl acetate in cyclohexane to afford the title compound as a white solid (47 mg). LCMS (A) m/z: 490 [M+1]⁺, Rt 1.55 min (acidic).

### EXAMPLE 1

### 5-(5-{3-Chloro-4-[(1-methylethyl)oxy]phenyl}-1,2,4-oxadiazol-3-yl)-1H-indazole

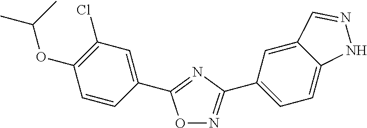

3-Chloro-4-[(1-methylethyl)oxy]benzoic acid (712 mg, 3.32 mmol) (Description 10), EDC (699 mg, 3.65 mmol) and HOST (493 mg, 3.65 mmol) were dissolved in DMF (16.5 mL) and the mixture stirred for 10 min. N-hydroxy-1H-indazole-5-carboximidamide (584 mg, 3.32 mmol) (Description 53) was then added and reaction mixture heated at 80° C. for 12 h and then left to stand at RT over the weekend. The mixture was then reduced in vacuo and the residue dissolved in water and extracted into ethyl acetate (×3). The organic layers were combined, washed with brine and reduced in vacuo to afford a solid. DCM was added and the mixture sonicated for 4 min then filtered and dried. This procedure was repeated twice more and the resulting solid triturated twice with ethanol to afford the title compound as a white solid (36 mg). LCMS (D) m/z: 355 [M+1]⁺, Rt 3.64 min.

### EXAMPLE 2

### 5-[3-(1H-Indazol-5-yl)-1,2,4-oxadiazol-5-yl]-2-[(1-methylethyl)oxy]benzonitrile

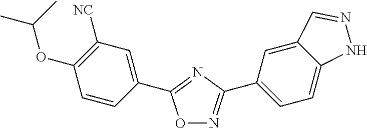

To a solution of 3-cyano-4-[(1-methylethyl)oxy]benzoic acid (291 mg, 1.419 mmol) (Description 7) in DMF (4 mL) was added EDC (299 mg, 1.561 mmol) and the reaction stirred at RT for 40 min under nitrogen. N-Hydroxy-1H-indazole-5-carboximidamide (250 mg, 1.419 mmol) (Description 53) was added and the reaction mixture stirred at RT for 16 h under nitrogen, followed by 80° C. for 16 h. The mixture was diluted with ethyl acetate (30 mL) and washed with saturated aqueous sodium bicarbonate solution (30 mL). The aqueous phase was further extracted with ethyl acetate (2×30 mL) and the organics combined, dried over a phase separation cartridge and reduced in vacuo. Purification by chromatography on silica gel, eluting with a gradient of 0-50% ethyl acetate in isohexane afforded an off-white solid (150 mg). 30 mg of this material was purified by MDAP (acidic conditions) to afford the title compound as a white solid (15 mg). LCMS (A) m/z: 346 [M+1]⁺, Rt 1.30 min (acidic), Rt 1.30 min (basic).

### EXAMPLE 3

### 5-[3-(1H-Indazol-4-yl)-1,2,4-oxadiazol-5-yl]-2-[(1-methylethyl)oxy]benzonitrile

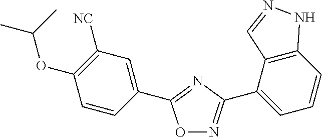

To a solution of 3-cyano-4-[(1-methylethyl)oxy]benzoic acid (864 mg, 4.2 mop (Description 7) in DMF (20 mL) was added EDC (1.2 g, 6.3 mmol) and HOBT (850 mg, 6.3 mmol) and the reaction stirred at RT for 30 min. N-Hydroxy-1H-indazole-4-carboximidamide (742 mg, 4.2 mmol) (Description 54) in DMF (20 mL) was then added and the reaction mixture stirred at 80° C. for 2 h, cooled over weekend, then reheated and more EDC (0.6 g, 3.1 mmol) added and heating continued. The mixture was diluted with ethyl acetate and saturated aqueous sodium bicarbonate solution and the layers separated. The aqueous layer was re-extracted and the organics combined, washed with brine, dried and reduced to afford a gum. Purification by chromatography on silica gel, eluting with ethyl acetate in hexane afforded a solid which was triturated with ether to afford the title compound as a cream solid (34 mg). LCMS (D) m/z: 346 [M+1]⁺, Rt 3.30 min.

### EXAMPLE 4

### 4-(5-{3-Chloro-4-[(1-methylethyl)oxy]phenyl}-1,2,4-oxadiazol-3-yl)-1H-indazole

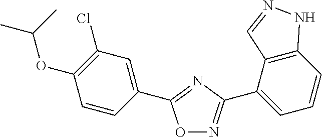

A mixture of 3-chloro-4-[(1-methylethyl)oxy]benzoic acid (0.15 g, 0.699 mmol) (Description 10), 1,1-dimethylethyl 4-[(hydroxyamino)(imino)methyl]-1H-indazole-1-carboxylate (0.193 g, 0.699 mmol) (Description 55), HOBT (0.118 g, 0.769 mmol) and EDC (0.147 g, 0.769 mmol) in DMF (4 mL) was stirred and heated at 80° C. for 6 h, then 120° C. for 2 h. The mixture was then cooled, diluted with ethyl acetate (30 mL) and water (30 mL) and the organic layer washed with water (3×15 mL), dried over magnesium sulfate and reduced. Purification by chromatography on silica gel eluting with 40% ethyl acetate in hexane afforded a white solid which was triturated with diethyl ether to afford the title compound as a white solid (22 mg). LCMS (D) m/z: 355 [M+1]⁺, Rt 3.77 min.

### Alternative:

To a solution of 3-chloro-4-[(1-methylethyl)oxy]benzoic acid (13.94 g, 65 mmol) (Description 10) in THF (100 mL) was added EDC (14.85 g, 65 mmol), HOBT (8.78 g, 65 mmol) and triethylamine (13.2 mL, 100 mL) and the mixture stirred at RT for 30 min. N-Hydroxy-1H-indazole-4-carboximidamide (8.8 g, 50 mmol) (Description 54) was added and the mixture stirred at RT overnight. TBAF (52.2 g, 200 mmol) was added and the mixture stirred at 80° C. for 3 days. The mixture was the reduced in vacuo and the residue was purified by chromatography on silica gel to afford the title compound (5.6 g).

### Alternative:

A mixture of 3-chloro-4-[(1-methylethyl)oxy]benzoyl chloride (1364 mg, 5.85 mmol) (Description 11) in acetonitrile (5 mL) was added to a cold suspension (0° C.) of N-hydroxy-1H-indazole-4-carboximidamide (1031 mg, 5.85 mmol) (Description 54) and triethylamine (0.897 mL, 6.44 mmol) in acetonitrile (25 mL) under argon. The reaction mixture was warmed to RT over 1 h and then heated at reflux overnight. The mixture was allowed to cool to RT for 1 night, then refluxed overnight, and then left to cool overnight and then to stand over the weekend. The resulting white precipitate was treated with ethyl acetate (30 mL) and water (10 mL) and the mixture was filtered, washing with ethyl acetate. The organic phase was separated and washed with brine, dried over sodium sulfate and reduced to give a pale brown solid which was dried in an oven overnight. Trituration with methanol with sonication was followed by filtration to give a white solid which was dried in the oven to afford the title compound (583 mg).

### EXAMPLE 5

### 3-[4-(5-{3-Chloro-4-[(1-methylethyl)oxy]phenyl}-1,2,4-oxadiazol-3-yl)-1H-indazol-1-yl]-2-methylpropanoic acid

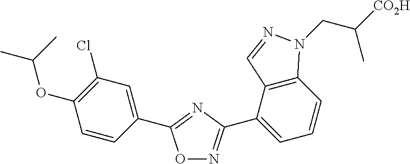

Aqueous sodium hydroxide (2M) (0.5 mL, 1.0 mmol) was added to a solution of methyl 3-[4-(5-{3-chloro-4-[(1-methylethyl)oxy]phenyl}-1,2,4-oxadiazol-3-yl)-1H-indazol-1-yl]-2-methylpropanoate (70 mg, 0.154 mmol) (Description 69) in ethanol (2 mL) and the mixture heated to 85° C. under argon overnight. The mixture was then allowed to cool to RT and the solvent reduced to give a white solid. Ethanol (2 mL) was added followed by hydrochloric acid (5M) (ca. 1 mL) until a white cloudy precipitate formed. This mixture was sonicated for 1 h then filtered and dried in the vacuum oven over the weekend to afford the title compound as a white solid (70 mg). LCMS (B) m/z: 441 [M+1]⁺, Rt 1.40 min.

### EXAMPLE 6

### 5-[4-(5-{3-Chloro-4-[(1-methylethyl)oxy]phenyl}-1,2,4-oxadiazol-3-yl)-1H-indazol-1-yl]pentanoic acid

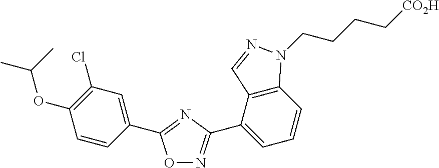

Sodium hydroxide (2M) (0.5 mL, 1.000 mmol) was added to a solution of methyl 5-[4-(5-{3-chloro-4-[(1-methylethyl)oxy]phenyl}-1,2,4-oxadiazol-3-yl)-1H-indazol-1-yl]pentanoate (54 mg, 0.115 mmol) (Description 70) in ethanol (2 mL) and the mixture heated to 65° C. under nitrogen for 6 h. The mixture was allowed to cool to RT and stand overnight under nitrogen. The solvent was then reduced to give a pale yellow solution which was acidified to form a white precipitate which was collected by filtration washing with cyclohexane. The filtrate was reduced and methanol (0.5 mL) and cyclohexane (2 mL) were added, the mixture filtered and the solid collected. Some solid precipitated on the funnel stem and receiver flask and this was recovered and dissolved in methanol and loaded onto an Isolute amine resin column, washed with ethanol and flushed with acetic acid. The ethanol phase was concentrated and filtered again through an Isolute amine column with ethanol and acetic acid. The two acid eluted phases were combined and reduced to give a colourless oil which solidified to a white solid which was dried in the vacuum oven overnight. This material was combined with the solid material obtained from the two filtrations to give the title compound as a white solid (50 mg). LCMS (B) m/z: 455 [M+1]⁺, Rt 1.41 min.

### EXAMPLE 7

### 5-[4-(5-{3-Chloro-4-[(1-methylethyl)oxy]phenyl}-1,2,4-oxadiazol-3-yl)-2H-indazol-2-yl]pentanoic acid

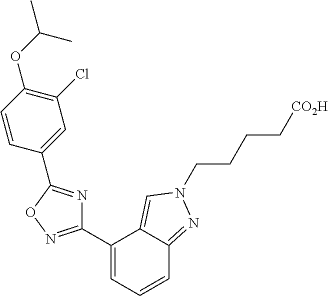

Sodium hydroxide (2M) (0.5 mL, 1.000 mmol) was added to a solution of methyl 5-[4-(5-{3-chloro-4-[(1-methylethyl)oxy]phenyl}-1,2,4-oxadiazol-3-yl)-2H-indazol-2-yl]pentanoate (54 mg, 0.115 mmol) (Description 71) in ethanol (2 mL) and the mixture heated to 65° C. under nitrogen for 6 h. The mixture was allowed to cool to RT and stand overnight. The solvent was reduced and ethanol (2 mL) and hydrochloric acid (5N) (0.5 mL) were added to give a white precipitate. The solvent was reduced to give a white solid, which was suspended in DCM/ethanol and loaded onto an Isolute amine column and flushed with ethanol and then acetic acid. The ethanol wash was reduced and flushed through an Isolute amine column in the same way. The combined acetic acid solutions were reduced to give a white solid which was re-suspended and reduced from DCM and dried in the vacuum oven overnight to afford the title compound (49 mg). LCMS (B) m/z: 455 [M+1]⁺, Rt 1.36 min.

Example 8: {3-[4-(5-{3-Chloro-4-[(1-methylethyl)oxy]phenyl}-1,2,4-oxadiazol-3-yl)-1H-indazol-1-yl]-3-oxetanyl}acetic acid

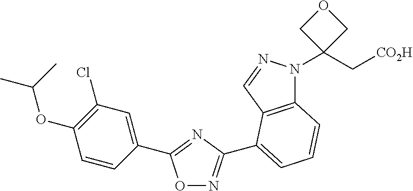

Sodium hydroxide (2M) (0.5 mL, 1.000 mmol) was added to a solution of ethyl {3-[4-(5-{3-chloro-4-[(1-methylethyl)oxy]phenyl}-1,2,4-oxadiazol-3-yl)-1H-indazol-1-yl]-3-oxetanyl}acetate (Description 72) (30 mg, 0.060 mmol) in ethanol (2 mL) and the mixture heated to 65° C. under nitrogen for 4 h. The mixture was allowed to cool to RT and was then reduced to give an off-white solid which was suspended in ethanol (2 mL) and hydrochloric acid (5M) (ca. 0.5 mL) added to give a white precipitate. This was filtered through filter paper and the filtrate reduced in volume and loaded onto an Isolute amine column which was washed with ethanol and then flushed with acetic acid. The acid phase was reduced to give a colourless oil which was dried under vacuum overnight to afford the title compound as a white crystalline solid (26 mg). LCMS (B) m/z: 469 [M+1]⁺, Rt 1.34 min.

### EXAMPLE 9

### Lithium 3-[4-(5-{3-chloro-4-[(1-methylethyl)oxy]phenyl}-1,2,4-oxadiazol-3-yl)-1H-indazol-1-yl]-2-hydroxypropanoate

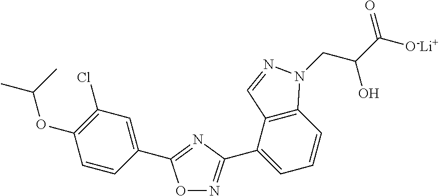

Lithium hydroxide (2.54 mg, 0.106 mmol) was added to a solution of ethyl 3-[4-(5-{3-chloro-4-[(1-methylethyl)oxy]phenyl}-1,2,4-oxadiazol-3-yl)-1H-indazol-1-yl]-2-hydroxypropanoate (50 mg, 0.106 mmol) (Description 74) in THF (1 mL), methanol (1 mL) and water (0.5 mL). The reaction was heated at 75° C. under nitrogen for 3 h. The mixture was allowed to cool and the solvent was then reduced to afford the title compound as a white solid (47 mg). LCMS (B) m/z: 443 [M+1]⁺, Rt 1.28 min. The stereochemistry of this compound was not determined.

### EXAMPLE 10

### 3-[5-(5-{5-Chloro-6-[(1-methylethyl)oxy]-3-pyridinyl}-1,2,4-oxadiazol-3-yl)-1H-indazol-1-yl]propanoic acid

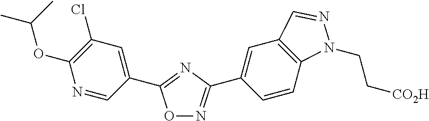

Ethyl 3-[5-(5-{5-chloro-6-[(1-methylethyl)oxy]-3-pyridinyl}-1,2,4-oxadiazol-3-yl)-1H-indazol-1-yl]propanoate (200 mg, 0.439 mmol) (Description 75) was dissolved in ethanol (4 mL) and THF (1 mL). The mixture was heated at 50° C. for 10 min until the suspension was fully soluble. The mixture was then cooled to RT, sodium hydroxide (2M) (2.193 mL, 4.39 mmol) added and the mixture stirred at RT for 30 min. The mixture was then reduced in vacuo and the residue re-dissolved in ethyl acetate (ca. 30 mL), washed with water (ca. 20 mL) and the aqueous phase acidified with hydrochloric acid (1M) until ca. pH 2 was obtained. The mixture was then extracted with ethyl acetate (3×20 mL) and the organic phases combined, dried over magnesium sulfate, filtered and reduced in vacuo to give a light yellow solid. Trituration with diethyl ether (ca. 10 mL) followed by filtration afforded a white solid which was dried in a vacuum oven at 40° C. overnight to afford the title compound (124 mg). LCMS (A) m/z: 428 [M+1]⁺, Rt 1.42 min (acidic); m/z: 426 [M+1]⁺, Rt 0.97 min (basic).

### EXAMPLE 11

### 5-(5-{5-Chloro-6-[(1-methylethyl)oxy]-3-pyridinyl}-1,2,4-oxadiazol-3-yl)-1H-indazole

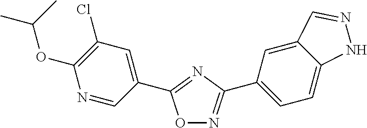

To a solution of 5-chloro-6-[(1-methylethyl)oxy]-3-pyridinecarboxylic acid (9.55 g, 44.27 mmol) (Description 13) in THF (100 mL) was added EDC (10.12 g, 44.27 mmol), HOBT (5.98 g, 44.27 mmol) and triethylamine (9 mL, 68.12 mmol) and the mixture stirred overnight at RT for 30 min. N-Hydroxy-1H-indazole-5-carboximidamide (6.0 g, 34.06 mmol) (Description 53) was added and the mixture stirred at RT overnight. TBAF (35.6 g, 136.24 mmol) was then added and the mixture stirred at 80° C. for 24 h. The mixture was the reduced in vacuo and partitioned between ethyl acetate and water and the organic layer dried over sodium sulfate, reduced in vacuo and purified by chromatography on silica gel to afford the title compound (6.101 g). LCMS 11k: 356 [M+1]⁺, Rt 1.589 min.

### EXAMPLE 12

### 4-[5-(5-{5-Chloro-6-[(1-methylethyl)oxy]-3-pyridinyl}-1,2,4-oxadiazol-3-yl)-6-methyl-1H-indazol-1-yl]butanoic acid

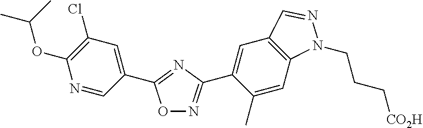

A mixture of ethyl 4-[5-(5-{5-chloro-6-[(1-methylethyl)oxy]-3-pyridinyl}-1,2,4-oxadiazol-3-yl)-6-methyl-1H-indazol-1-yl]butanoate (66 mg, 0.136 mmol) (Description 76) and sodium hydroxide (2M) (0.3 mL, 0.600 mmol) in THF (2 mL) was stirred at RT for 2.5 h. Methanol (4 mL) and additional sodium hydroxide (2M) (0.3 mL, 0.600 mmol) were then added and the mixture stirred at RT for 3.5 h. The resulting mixture was reduced to near dryness, diluted with DCM (10 mL) and water (5 mL) and then adjusted to ca. pH 2 with hydrochloric acid (1M). The aqueous layer was separated and extracted with ethyl acetate (4×10 mL) and the combined organics passed through a phase separator, reduced, then purified by MDAP (acidic conditions) to afford the title compound as a white solid (40 mg). LCMS (A) m/z: 456 [M+1]⁺, Rt 1.47 min (acidic), Rt 1.06 min (basic).

### EXAMPLE 13

### 4-[5-(5-{5-Chloro-6-[(1-methylethyl)oxy]-3-pyridinyl}-1,2,4-oxadiazol-3-yl)-4-methyl-1H-indazol-1-yl]butanoic acid

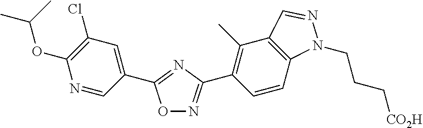

Sodium hydroxide (2M) (0.188 mL, 0.376 mmol) was added to a solution of ethyl 4-[5-(5-{5-chloro-6-[(1-methylethyl)oxy]-3-pyridinyl}-1,2,4-oxadiazol-3-yl)-4-methyl-1H-indazol-1-yl]butanoate (91 mg, 0.188 mmol) (Description 77) in THF (2 mL) and the mixture stirred for ca. 40 h at RT. The mixture was adjusted to ca. pH 2 with hydrochloric acid (1M), then concentrated to dryness and purified by MDAP (acidic conditions) to afford the title compound as a white solid (40 mg). LCMS (A) m/z: 456 [M+1]⁺, Rt 1.47 min (acidic), Rt 1.07 min (basic).

### EXAMPLE 14

### 4-[5-(5-{5-Cyano-6-[(1-methylethyl)oxy]-3-pyridinyl}-1,2,4-oxadiazol-3-yl)-1H-indazol-1-yl]butanoic acid

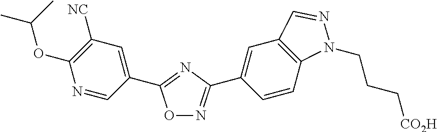

Ethyl 4-[5-(5-{(5-cyano-6-[(1-methylethyl)oxy]-3-pyridinyl}-1,2,4-oxadiazol-3-yl)-1H-indazol-1-yl]butanoate (46 mg, 0.100 mmol) (Description 78) in isopropanol (3 mL) was heated to 40° C. and THF (2 mL) added. The solution was allowed to cool to RT and sodium hydroxide (2M) (0.060 mL, 0.120 mmol) was added. The reaction mixture was stirred at RT overnight and then additional sodium hydroxide (2M) (0.025 mL, 0.050 mmol) was added and the mixture left to stir for 3 h 15 min. The reaction mixture was then reduced in vacuo, and the resulting solid re-dissolved in water, acidified to pH 4 using acetic acid and extracted with ethyl acetate (×3). The organic layers were combined, dried over a phase separator and reduced in vacuo to give a pale orange solid which was dried under high vacuum (50 mg). This material was combined with additional material (30 mg) obtained from a similar reaction and then purified by MDAP (acidic conditions) to afford a white solid which was dried under high vacuum to give the title compound (24 mg). LCMS (A) m/z: 433 [M+1]⁺, Rt 1.30 min (acidic), Rt 0.91 min (basic).

### EXAMPLE 15

### 5-(5-{5-Chloro-6-[(1-methylethyl)oxy]-3-pyridinyl}-1,2,4-oxadiazol-3-yl)-6-methyl-1H-indazole

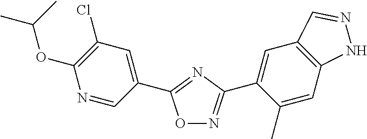

A solution of 5-chloro-6-[(1-methylethyl)oxy]-3-pyridinecarboxylic acid (206 mg, 0.957 mmol) (Description 13), HATU (437 mg, 1.148 mmol) and DIPEA (0.201 mL, 1.148 mmol) in DMF (2 mL) was stirred at RT for 15 min. A solution of N-hydroxy-6-methyl-1H-indazole-5-carboximidamide (182 mg, 0.957 mmol) (Description 61) in DMF (6 mL) was then added drop-wise and the mixture heated at 80° C. for 16 h then stirred at RT for 24 h. The mixture was then reduced in vacuo and the residue purified by chromatography on silica gel eluting with a gradient of 0-30% ethyl acetate in isohexane. The resulting product was triturated with a mixture of methanol (ca. 4 mL) and ether (ca. 1 mL), filtered and the solid dried to afford the title compound as a white solid (70 mg). LCMS (A) m/z: 370 [M+1]⁺, Rt 1.50 min (acidic), Rt 1.51 min (basic).

### EXAMPLE 16

### 5-(5-{5-Chloro-6-[(1-methylethyl)oxy]-3-pyridinyl}-1,2,4-oxadiazol-3-yl)-6-fluoro-1H-indazole

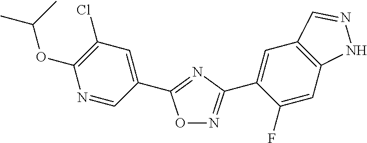

A mixture of 5-chloro-6-[(1-methylethyl)oxy]-3-pyridinecarboxylic acid (325 mg, 1.507 mmol) (Description 13), HATU (688 mg, 1.809 mmol), and DIPEA (0.316 mL, 1.809 mmol) in DMF (12 mL) was stirred at RT for 15 min. 6-Fluoro-N-hydroxy-1H-indazole-5-carboximidamide (351 mg, 1.809 mmol) (Description 62) was then added portion-wise and the mixture heated at 80° C. overnight. The mixture was then reduced in vacuo and the residue partitioned between water (15 mL) and DCM (40 mL). The aqueous layer was separated and further extracted with DCM (4×15 mL) the organics combined, reduced and purified by chromatography on silica gel eluting with a gradient of 0-40% ethyl acetate in isohexane. The resulting product was triturated with a mixture of methanol and ether, filtered and the solid dried at 60° C. for 2 days to afford the title compound as an off-white solid (185 mg). LCMS (A) m/z: 374 [M+1]⁺, Rt 1.44 min (acidic), Rt 1.45 min (basic).

### EXAMPLE 17

### 4-[5-(5-{5-Chloro-6-[(1-methylethyl)oxy]-3-pyridinyl}-1,2,4-oxadiazol-3-yl)-6-fluoro-1H-indazol-1-yl]butanoic acid

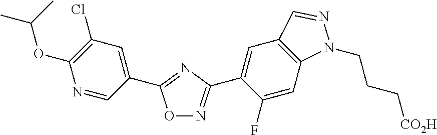

A mixture of ethyl 4-[5-(5-{5-chloro-6-[(1-methylethyl)oxy]-3-pyridinyl}-1,2,4-oxadiazol-3-yl)-6-fluoro-1H-indazol-1-yl]butanoate (117 mg, 0.240 mmol) (Description 79) and aqueous sodium hydroxide (2M) (0.360 mL, 0.719 mmol) in THF (2.5 mL) was stirred at RT overnight. Methanol (3 mL) was then added and the mixture stirred for 3 h at RT. Additional aqueous sodium hydroxide (2M) (0.240 mL, 0.480 mmol) was added and stirring continued at RT for an additional 6 h. The mixture was then reduced to near dryness, partitioned between ethyl acetate (20 mL) and water (10 mL), adjusted to ca. pH 2 with aqueous hydrochloric acid (2M) and the aqueous separated and further extracted with ethyl acetate (3×10 mL). The organics were combined, reduced and purified by chromatography on silica gel eluting with a gradient of 0-10% methanol in DCM to afford the title compound as a white solid (69 mg). LCMS (A) m/z: 460 [M+1]⁺, Rt 1.44 min (acidic), Rt 1.13 min (basic).

### EXAMPLE 18

### 4-[5-(5-{5-Chloro-6-[(1-methylethyl)oxy]-3-pyridinyl}-1,2,4-oxadiazol-3-yl)-6-fluoro-2H-indazol-2-yl]butanoic acid

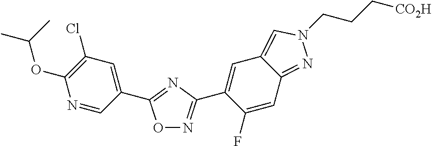

A mixture containing ethyl 4-[5-(5-{5-chloro-6-[(1-methylethyl)oxy]-3-pyridinyl}-1,2,4-oxadiazol-3-yl)-6-fluoro-2H-indazol-2-yl]butanoate (72 mg, 0.148 mmol) (Description 80) and aqueous sodium hydroxide (2M) (0.295 mL, 0.590 mmol) in methanol (6 mL) was stirred at RT overnight. Additional aqueous sodium hydroxide (2M) (0.74 mL, 1.48 mmol) was added and the mixture stirred overnight at RT. The mixture was then reduced to near dryness and partitioned between ethyl acetate (20 mL) and water (10 mL), and adjusted to ca. pH 2 with aqueous hydrochloric acid (2M). The aqueous layer was separated and further extracted ethyl acetate (3×10 mL) and the combined organics reduced, and the residue triturated with a mixture of diethyl ether (5 mL) and ethyl acetate (1 mL) then filtered. The resulting solid was dried at 40° C. in a vacuum oven over the weekend to afford the title compound as a white solid (30 mg). LCMS (A) m/z: 460 [M+1]⁺, Rt 1.39 min (acidic), Rt 1.07 min (basic).

### EXAMPLE 19

### 6-Chloro-5-(5-{5-chloro-6-[(1-methylethyl)oxy]-3-pyridinyl}-1,2,4-oxadiazol-3-yl)-1H-indazole

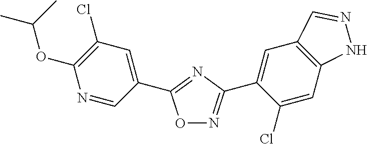

A mixture of 5-chloro-6[(1-methylethyl)oxy]-3-pyridinecarboxylic acid (400 mg, 1.855 mmol) (Description 13), HATU (757 mg, 1.992 mmol) and DIPEA (0.348 mL, 1.992 mmol) in DMF (5 mL) was stirred at RT for 15 min. A solution of 6-chloro-N-hydroxy-1H-indazole-5-carboximidamide (666 mg, 1.897 mmol) (Description 63) in DMF (5 mL) was then added drop-wise and the mixture heated at 80° C. for ca. 18 h. The mixture was then reduced to near dryness and the residue partitioned between ethyl acetate (ca. 30 mL) and water (ca. 10 mL). The aqueous layer was separated and further extracted with ethyl acetate (2×10 mL) and the organics combined, reduced and the residue purified by chromatography on silica gel eluting with a gradient of 0-40% ethyl acetate in isohexane. The resulting product was triturated with a minimal volume of methanol, filtered and the resulting solid dried at 50° C. in a vacuum oven overnight to afford the title compound as a light orange solid (130 mg). LCMS (A) m/z: 390 [M+1]⁺, Rt 1.48 min (acidic), Rt 1.49 min (basic).

### EXAMPLE 20

### 4-[6-Chloro-5-(5-{5-chloro-6-[(1-methylethyl)oxy]-3-pyridinyl}-1,2,4-oxadiazol-3-yl)-1H-indazol-1-yl]butanoic acid

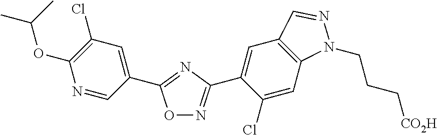

A mixture of ethyl 4-[6-chloro-5-(5-{5-chloro-6-[(1-methylethyl)oxy]-3-pyridinyl}-1,2,4-oxadiazol-3-yl)-1H-indazol-1-yl]butanoate (90 mg, 0.178 mmol) (Description 81) and aqueous sodium hydroxide (2M) (0.178 mL, 0.357 mmol) in methanol (5 mL) was stirred at RT over the weekend. Additional aqueous sodium hydroxide (2M) (0.900 mL, 1.800 mmol) was added and the mixture stirred at RT for another 2 days. The mixture was then reduced to near dryness, partitioned between ethyl acetate (20 mL) and water (10 mL), and adjusted to ca. pH 2 with aqueous hydrochloric acid (2M). The aqueous was separated and extracted with ethyl acetate (3×10 mL) and the combined organics reduced. The residue was then triturated with ether containing a minimal amount of methanol, filtered and dried to give the title compound as a white solid (66 mg). LCMS (A) m/z: 476 [M+1]⁺, Rt 1.47 min (acidic), Rt 1.08 min (basic).

### EXAMPLE 21

### 4-[6-Chloro-5-(5-{5-chloro-6-[(1-methylethyl)oxy]-3-pyridinyl}-1,2,4-oxadiazol-3-yl)-2H-indazol-2-yl]butanoic acid

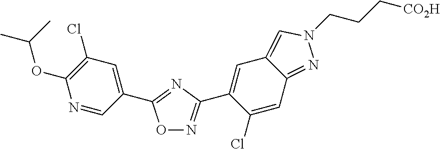

A mixture of ethyl 4-[6-chloro-5-(5-{5-chloro-6-[(1-methylethyl)oxy]-3-pyridinyl}-1,2,4-oxadiazol-3-yl)-2H-1-indazol-2-yl]butanoate (53 mg, 0.105 mmol) (Description 82) and aqueous sodium hydroxide (2M) (0.158 mL, 0.315 mmol) in methanol (5 mL) was stirred at RT over the weekend. Additional aqueous sodium hydroxide (2M) (1.000 mL, 2.000 mmol) was added and the mixture stirred at RT for another 2 days. The mixture was then reduced to near dryness, partitioned between ethyl acetate (20 mL) and water (10 mL), and adjusted to ca. pH 2 with aqueous hydrochloric acid (2M). The aqueous was separated and extracted with ethyl acetate (3×10 mL) and the combined organics reduced. The residue was then triturated with ether, filtered and dried to give the title compound as a white solid (5.5 mg). LCMS (A) m/z: 476 [M+1]⁺, Rt 1.42 min (acidic), Rt 1.06 min (basic).

### EXAMPLE 22

### 5-(5-{5-Chloro-6-[(1-methylethyl)oxy]-3-pyridinyl}-1,2,4-oxadiazol-3-yl)-6-ethyl-1H-indazole

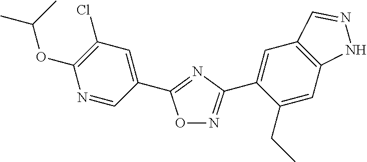

A mixture of 5-chloro-6-[(1-methylethyl)oxy]-3-pyridinecarboxylic acid (298 mg, 1.383 mmol) (Description 13), HATU (526 mg, 1.383 mmol), and DIPEA (0.242 mL, 1.383 mmol) in DMF (4 mL) was stirred at RT for 15 min. A solution of 6-ethyl-N-hydroxy-1H-indazole-5-carboximidamide (277 mg, 1.153 mmol) (Description 64) in DMF (2 mL) was then added drop-wise and the mixture heated at 80° C. overnight. The mixture was then reduced to near dryness and the residue purified twice by chromatography on silica gel eluting with a gradient of 0-30% ethyl acetate in isohexane. The resulting product was then triturated with a minimal volume of methanol, filtered and dried to afford the title compound as an off-white solid (140 mg). LCMS (A) m/z: 384 [M+1]⁺, Rt 1.58 min (acidic), Rt 1.59 min (basic).

### EXAMPLE 23

### 4-[5-(5-{5-Chloro-6-[(1-methylethyl)oxy]-3-pyridinyl}-1,2,4-oxadiazol-3-yl)-6-ethyl-2H-indazol-2-yl]butanoic acid

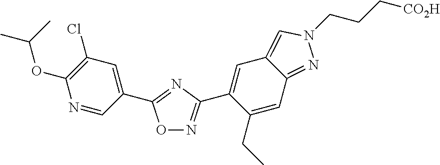

A mixture of ethyl 4-[5-(5-{5-chloro-6-[(1-methylethyl)oxy]-3-pyridinyl}-1,2,4-oxadiazol-3-yl)-6-ethyl-2H-indazol-2-yl]butanoate (58 mg, 0.116 mmol) (Description 84), aqueous sodium hydroxide (2M) (3 mL, 6.00 mmol) in methanol (5 mL) was stirred at RT for ca. 20 h. The mixture was then reduced to near dryness, partitioned between ethyl acetate (20 mL) and water (10 mL), then adjusted to ca. pH 2 with aqueous hydrochloric (2M). The aqueous was separated and extracted with ethyl acetate (3×10 mL) and the combined organics reduced. The residue was then triturated with ether, filtered and dried to give the title compound as a white solid (31 mg). LCMS (A) m/z: 470 [M+1]⁺, Rt 1.48 min (acidic), Rt 1.13 min (basic).

### EXAMPLE 24

### 4-[5-(5-{5-Chloro-6-[(1-methylethyl)oxy]-3-pyridinyl}-1,2,4-oxadiazol-3-yl)-6-ethyl-1H-indazol-1-yl]butanoic acid

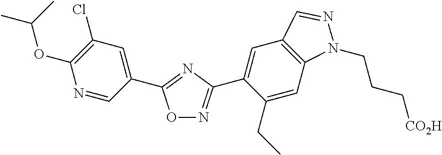

A mixture of ethyl 4-[5-(5-{5-chloro-6-[(1-methylethyl)oxy]-3-pyridinyl}-1,2,4-oxadiazol-3-yl)-6-ethyl-1H-indazol-1-yl]butanoate (136 mg, 0.273 mmol) (Description 83) and aqueous sodium hydroxide (2M) (1.4 mL, 2.80 mmol) in methanol (5 mL) was stirred at RT for ca. 22 h. The mixture was then reduced to near dryness, partitioned between ethyl acetate (20 mL) and water (10 mL), then adjusted to ca. pH 2 with aqueous hydrochloric (2M). The aqueous was separated and extracted ethyl acetate (3×10 mL) and the combined organics reduced. The residue was purified by MDAP (acidic conditions) to afford the title compound as a white solid (72 mg). LCMS (A) m/z: 470 [M+1]⁺, Rt 1.52 min (acidic), Rt 1.13 min (basic).

### EXAMPLE 25

### 5-(5-{5-Chloro-6-[(1-methylethyl)oxy]-3-pyridinyl}-1,2,4-oxadiazol-3-yl)-6-(methyloxy)-1H-indazole

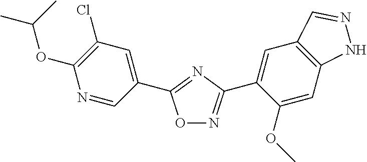

A mixture of 5-chloro-6-[(1-methylethyl)oxy]-3-pyridinecarboxylic acid (213 mg, 0.989 mmol) (Description 13), HATU (376 mg, 0.989 mmol), and DIPEA (0.173 ml, 0.989 mmol) in DMF (4 mL) was stirred at RT for 15 min. A solution of N-hydroxy-6-(methyloxy)-1H-indazole-5-carboximidamide (170 mg, 0.824 mmol) (Description 65) in DMF (2 mL) was then added drop-wise and the mixture heated at 80° C. overnight. After cooling, the mixture was reduced to dryness and the residue purified by MDAP (acidic conditions) to afford a yellow solid which was further purified by HPLC (CHIRALPAK IA (250×20 mm-5 micron); isohexane/ethanol gradient 65:35, 18 ml/min) to afford the title compound as a light brown solid (57 mg). LCMS (A) m/z: 386 [M+1]⁺, Rt 1.37 min (acidic), Rt 1.62 min (basic).

### EXAMPLE 26

### 3-[5-(5-{5-Cyano-6-[(1-methylethyl)oxy]-3-pyridinyl}-1,2,4-oxadiazol-3-yl)-1H-indazol-1-yl]propanoic acid

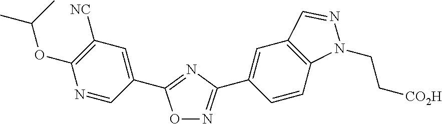

To a solution of ethyl 3-[5-(5-{5-cyano-6-[(1-methylethyl)oxy]-3-pyridinyl}-1,2,4-oxadiazol-3-yl)-1H-indazol-1-yl]propanoate (190 mg, 0.426 mmol) (Description 85) in THF (2 mL) and isopropanol (2 mL) was added aqueous sodium hydroxide (2M) (0.532 mL, 1.064 mmol) and the reaction mixture stirred at RT under nitrogen for 16 h. The mixture was then reduced to dryness and the residue taken up in water (30 mL) and acidified to pH 4 using acetic acid before extracting with DCM (3×20 mL). The organics were combined, dried over a phase separation cartridge and reduced in vacuo to afford an off-white solid which was triturated with diethyl ether (2×5 mL). Further purification by MDAP (acidic conditions) afforded the title compound as a white solid (64 mg). LCMS (A) m/z: 419 [M+1]⁺, Rt 1.28 min (acidic), m/z: 417 [M−1]⁺, Rt 0.98 min (basic).

### EXAMPLE 27

### 4-[6-Chloro-5-(5-{5-cyano-6-[(1-methylethyl)oxy]-3-pyridinyl}-1,2,4-oxadiazol-3-yl)-1H-indazol-1-yl]butanoic acid

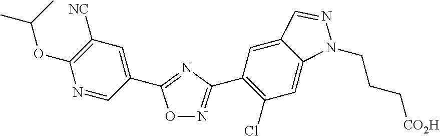

A mixture of ethyl 4-[6-chloro-5-(5-{5-cyano-6-[(1-methylethyl)oxy]-3-pyridinyl}-1,2,4-oxadiazol-3-yl)-1H-indazol-1-yl]butanoate (128.7 mg, 0.260 mmol) (Description 86) and aqueous sodium hydroxide (1M) (0.260 mL, 0.260 mmol) in isopropanol (6 mL) was stirred overnight. Methanol (3 mL) and additional aqueous sodium hydroxide (1M) (0.26 mL) were then added and the mixture was stirred at RT for 1 h. Additional methanol (3 mL) and aqueous sodium hydroxide (1M) (0.52 mL) were then added, the mixture stirred at RT for another 1 h. The mixture was then reduced to dryness, diluted with water (15 mL), adjusted to pH 1 with aqueous hydrochloric acid (6M), and extracted with ethyl acetate (3×15 mL). The combined organics were reduced and purified by MDAP (acidic conditions) to afford the title compound as a white solid (37 mg). LCMS (A) m/z: 467 [M+1]⁺, Rt 1.37 min (acidic), Rt 1.09 min (basic).

### EXAMPLE 28

### 4-[6-Chloro-5-(5-{3-cyano-4-[(1-methylethyl)oxy]phenyl}-1,2,4-oxadiazol-3-yl)-1H-indazol-1-yl]butanoic acid

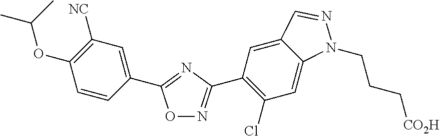

To a solution of ethyl 4-[6-chloro-5-(5-{3-cyano-4-[(1-methylethyl)oxy]phenyl}-1,2,4-oxadiazol-3-yl)-1H-indazol-1-yl]butanoate (72 mg, 0.146 mmol) (Description 87) in DCM (1 mL), THF (2 mL) and isopropanol (2 mL) was added aqueous sodium hydroxide (2M) (0.182 mL, 0.364 mmol) and the mixture stirred at RT overnight. Additional aqueous sodium hydroxide (2M) (0.15 mL, 0.30 mmol) was added and the mixture stirred at RT for another 4 h. The mixture was then reduced to dryness, diluted with water (15 mL), acidified with aqueous hydrochloric acid (6M), and extracted with ethyl acetate (3×15 mL). The combined organics were reduced and the residue triturated with a mixture of diethyl ether (ca. 10 mL), DCM (ca. 1 mL) and methanol (ca. 1 mL), filtered and the resulting solid dried in a vacuum oven to afford the title compound as a white solid (55 mg). LCMS (A) m/z: 466 [M+1]⁺, Rt 1.34 min (acidic), Rt 1.06 min (basic).

### EXAMPLE 29

### 4-[5-(5-{3-Cyano-4-[(1-methylethyl)oxy]phenyl}-1,2,4-oxadiazol-3-yl)-6-methyl-1H-indazol-1-yl]butanoic acid

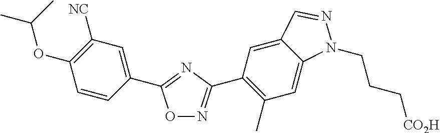

A mixture of ethyl 4-[5-(5-{3-cyano-4-[(1-methylethyl)oxy]phenyl}-1,2,4-oxadiazol-3-yl)-6-methyl-1H-indazol-1-yl]butanoate (136 mg, 0.287 mmol) (Description 88) and 2M aqueous sodium hydroxide (2M) (0.503 mL, 1.005 mmol) in isopropanol (2 mL) and THF (2 mL) was stirred at RT overnight. Methanol (5 mL) was then added and the mixture stirred at RT for another 3 h. The mixture was then reduced to dryness, diluted with water (10 mL), acidified with aqueous hydrochloric acid (6M), and extracted with ethyl acetate (3×15 mL). The combined organics were dried over magnesium sulfate, reduced and the residue triturated with diethyl ether (ca. 5 mL), filtered and the resulting solid dried in a vacuum oven to afford the title compound as a white solid (60 mg). LCMS (A) m/z: 446 [M+1]⁺, Rt 1.41 min (acidic), Rt 1.11 min (basic).

### EXAMPLE 30

### 4-[5-(5-{3-Chloro-4-[(1-methylethyl)oxy]phenyl}-1,2,4-oxadiazol-3-yl)-6-methyl-1H-indazol-1-yl]butanoic acid

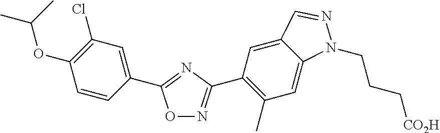

A mixture of ethyl 4-[5-(5-{3-chloro-4-[(1-methylethyl)oxy]phenyl}-1,2,4-oxadiazol-3-yl)-6-methyl-1H-indazol-1-yl]butanoate (143 mg, 0.296 mmol) (Description 89) and aqueous sodium hydroxide (2M) (0.6 mL, 1.200 mmol) in DCM (1 mL) and isopropanol (3 mL) was stirred at RT overnight. The mixture was then reduced to dryness, diluted with water (10 mL), acidified with aqueous hydrochloric acid (6M), and extracted with ethyl acetate (3×15 mL). The combined organics were dried over magnesium sulfate, reduced and the residue triturated with diethyl ether (ca. 5 mL), filtered and the resulting solid dried in a vacuum oven to afford the title compound as a white solid (93 mg). LCMS (A) m/z: 455 [M+1]⁺, Rt 1.03 min (basic).

### EXAMPLE 31

### 4-[5-(5-{3-Cyano-4-[(1-methylethyl)oxy]phenyl}-1,2,4-oxadiazol-3-yl)-6-ethyl-1H-indazol-1-yl]butanoic acid

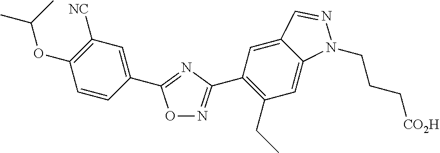

A mixture of ethyl 4-[5-(5-{(3-cyano-4-[(1-methylethyl)oxy]phenyl}-1,2,4-oxadiazol-3-yl)-6-ethyl-1H-indazol-1-yl]butanoate (65 mg, 0.133 mmol) (Description 90) and aqueous sodium hydroxide (1M) (0.33 ml, 0.330 mmol) in isopropanol (1 mL) and THF (2 mL) was stirred at RT overnight. Additional methanol (2 mL) and aqueous sodium hydroxide (1M) (0.6 mL, 0.60 mmol) was added and the mixture stirred at RT for another 3 h. The mixture was then reduced to dryness, diluted with water (15 mL), acidified with aqueous hydrochloric acid (6M), and extracted with ethyl acetate (3×15 mL). The combined organics were then reduced and the residue triturated with ether (ca. 5 mL) and methanol (ca. 0.5 mL), filtered and the resulting solid dried in a vacuum oven to afford the title compound as a white solid (45 mg). LCMS (A) m/z: 460 [M+1]⁺, Rt 1.57 min (acidic).

### EXAMPLE 32

### 4-[5-(5-{3-Cyano-4-[(1-methylethyl)oxy]phenyl}-1,2,4-oxadiazol-3-yl)-6-(methyloxy)-1H-indazol-1-yl]butanoic acid

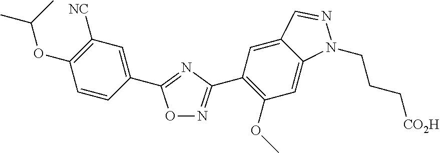

A mixture of ethyl 4-[5-(5-{3-cyano-4-[(1-methylethyl)oxy]phenyl}-1,2,4-oxadiazol-3-yl)-6-(methyloxy)-1H-indazol-1-yl]butanoate (46.5 mg, 0.095 mmol) (Description 91) and aqueous sodium hydroxide (2M) (0.190 ml, 0.380 mmol) in isopropanol (3 mL) and THF (2 mL) was stirred at RT overnight. The mixture was then reduced to dryness, diluted with water (10 mL), acidified with aqueous hydrochloric acid (1M), and extracted with ethyl acetate (3×15 mL). The organics were combined and reduced and the residue triturated with ether (ca. 1 mL) and methanol (ca. 3 mL), filtered and the resulting solid dried in a vacuum oven to afford the title compound as a white solid (29 mg). LCMS (A) m/z: 462 [M+1]⁺, Rt 1.01 min (basic).

### S1P1 Tango Assay

Recombinant EDG1-bla/U2OS cells (contain the human Endothelial Differentiation Gene 1 (EDG1) linked to a TEV protease site and a Gal4-VP16 transcription factor stably integrated into the Tango GPCR-bla U2OS parental cell line) were harvested from growth medium and passaged into assay medium (Invitrogen Freestyle Expression Medium). The cells were starved for 24 hours at 37° C., 5% CO₂, harvested and resuspended in assay medium at a density of ˜200,000 cells/ml. All test compounds were dissolved in DMSO at a concentration of 10 mM and were prepared in 100% DMSO to provide 10 point dose response curves. Test compounds prepared by Bravo (Velocity11) were added to wells in columns 2-11 and 13-22; DMSO was added to wells in columns 12 and 23 as unstimulated controls and assay medium was added to wells in columns 1 and 24 as cell-free controls. An S1P1 agonist was added to wells in row 2, columns 2-11 as stimulated controls and test compounds were added to wells in row 2, columns 13-22 and rows 3-15, columns 2-11/13-22 (row 1 and 16 were empty and not used). Compounds in solution were added to the assay plate (Greiner 781090) using an Echo (Labcyte) dose-response program (50 nl/well). The unstimulated and cell-free controls were loaded with 50 nl/well pure DMSO to ensure that the DMSO concentration was constant across the plate for all assays.

50 μl of the cell suspension was added to each well in columns 2-23 of the plate (˜10,000 cells per well). 50 μl of assay medium was added to each well in the cell-free controls (columns 1 and 24). The cells were incubated overnight at 37° C./5% CO₂. 10 μl of 6× substrate mixture (LiveBLAzer™-FRET BIG substrate (CCF4-AM) Cat # K1096 from Invitrogen, Inc.) was added to each well using Bravo and the plates incubated at room temperature for 2 h in the dark. The plate was finally read on EnVision for two emission channels (460 nm and 530 nm).

The blue/green emission ratio (460 nm/530 nm) was calculated for each well, by dividing the background-subtracted Blue emission values by the background-subtracted Green emission values. The dose response curve is based on sigmoidal dose-response model. All ratio data was normalized based upon the maximum emission ratio of positive control and minimum emission ratio of negative control (DMSO) on each plate. The intrinsic activity (IA) of each compound would be the normalized percentage of its maximum response after curve fitting.

Exemplified compounds of the invention had a pEC50>6. Examples 4, 5, 6, 8, 10, 11, and 17 had a pEC50 between 7 and 8. Examples 2, 9, 13 and 19 had a pEC50 between 8 and 9. Examples 1, 3 and 14 had a pEC50≧9.

### S1P3 GeneBlazer Assay

GeneBLAzer EDG3-Ga15-NFAT-bla HEK 293T cells (contain the human Endothelial Differentiation G-protein Coupled Receptor 3 (EDG3) and a beta-lactamase reporter gene under control of a NFAT response element and a promiscuous G Protein, Gal 5, stably integrated into the GeneBLAzer Ga15-NFAT-bla HEK 293T cell line) were suspended in assay medium (99% DMEM, 1% Dialyzed FBS, 0.1 mM NEAA, 25 mM HEPES (pH 7.3), 100 U/ml penicillin, 100 μg/ml streptomycin) at a density of 312, 500 cells/ml. Add 100 μl/well of the assay medium to the cell-free control wells (column 12) and 100 μl/well of the cell suspension to the test compound wells (row 2-8, column 1-10), the unstimulated control wells (DMSO) (column 11), and stimulated control wells (S1P) (row 1, column 1-10) in a Corning black-well, clear bottom 96-well plate. Cells were incubated at 37° C., 5% CO2 for 24 h.

Add 25 μl of 5× stock solution of test compounds in assay medium with 0.5% DMSO to the test compound wells, 25 μl of 5× stock solution of agonist (S1P) in assay medium with 0.5% DMSO to the stimulated compound wells, and 25 μl of 5× stock solution of 0.5% DMSO in assay medium to the unstimulated control and cell-free Control wells.

After incubation at 37° C., 5% CO2 for 5 h, 25 μl of 6× substrate mixture (6 μl Solution A (LiveBLAzer™-FRET BIG substrate (CCF4-AM) Cat # K1096 from Invitrogen, Inc.) was added to each well and incubate at room temperature for 2 h in dark. The plate was finally read on EnVision for two emission channels (460 nm and 530 nm).

All test compounds were dissolved in DMSO at a concentration of 10 mM and were prepared in 100% DMSO using a 1 in 5 dilution step to provide 10 point dose response curves. The dilutions were transferred to the assay plates ensuring that the DMSO concentration was constant across the plate for all assays.

Calculate the blue/green emission ratio (460 nm/530 nm) for each well, by dividing the background-subtracted Blue emission values by the background-subtracted green emission values. The dose response curve is based on sigmoidal dose-response model. All ratio data was normalized based upon the maximum emission ratio of positive control (S1P) and minimum emission ratio of negative control (DMSO) on each plate. The intrinsic activity (IA) of each compound would be the normalized percentage of its maximum response after curve fitting.

Exemplified compounds of the invention had a pEC50<5.

1. A compound of formula (I) or a salt thereof: 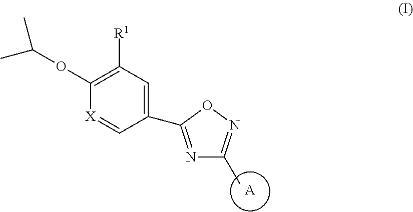
wherein
X is CH or N;
R¹ is chloro or cyano;
A is a bicyclic ring selected from: 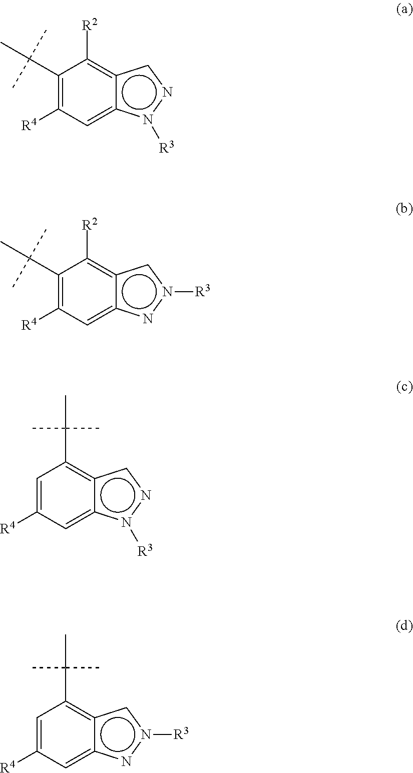
R² is hydrogen or methyl;
R³ is hydrogen, (CH₂)₂₋₄COOH, CH₂CH(CH₃)COOH, CH₂CH(OH)COOH or C(CH₂OCH₂)CH₂COOH; and
R⁴ is hydrogen, methyl, ethyl, fluoro, chloro or methoxy.

2. A compound selected from:
5-(5-{3-chloro-4-[(1-methylethyl)oxy]phenyl}-1,2,4-oxadiazol-3-yl)-1H-indazole
5-[3-(1H-indazol-5-yl)-1,2,4-oxadiazol-5-yl]-2-[(1-methylethyl)oxy]benzonitrile
5-[3-(1H-indazol-4-yl)-1,2,4-oxadiazol-5-yl]-2-[(1-methylethyl)oxy]benzonitrile
4-(5-{3-chloro-4-[(1-methylethyl)oxy]phenyl}-1,2,4-oxadiazol-3-yl)-1H-indazole
3-[4-(5-{3-chloro-4-[(1-methylethyl)oxy]phenyl}-1,2,4-oxadiazol-3-yl)-1H-indazol-1-yl]-2-methylpropanoic acid
5-[4-(5-{3-chloro-4-[(1-methylethyl)oxy]phenyl}-1,2,4-oxadiazol-3-yl)-1H-indazol-1-yl]pentanoic acid
5-[4-(5-{3-chloro-4-[(1-methylethyl)oxy]phenyl}-1,2,4-oxadiazol-3-yl)-2H-indazol-2-yl]pentanoic acid
{3-[4-(5-{3-chloro-4-[(1-methylethyl)oxy]phenyl}-1,2,4-oxadiazol-3-yl)-1H-indazol-1-yl]-3-oxetanyl}acetic acid
3-[4-(5-{3-chloro-4-[(1-methylethyl)oxy]phenyl}-1,2,4-oxadiazol-3-yl)-1H-indazol-1-yl]-2-hydroxypropanoic acid
3-[5-(5-{5-chloro-6-[(1-methylethyl)oxy]-3-pyridinyl}-1,2,4-oxadiazol-3-yl)-1H-indazol-1-yl]propanoic acid
5-(5-{5-chloro-6-[(1-methylethyl)oxy]-3-pyridinyl}-1,2,4-oxadiazol-3-yl)-1H-indazole
4-[5-(5-{5-chloro-6-[(1-methylethyl)oxy]-3-pyridinyl}-1,2,4-oxadiazol-3-yl)-6-methyl-1H-indazol-1-yl]butanoic acid
4-[5-(5-{5-chloro-6-[(1-methylethyl)oxy]-3-pyridinyl}-1,2,4-oxadiazol-3-yl)-4-methyl-1H-indazol-1-yl]butanoic acid
4-[5-(5-{5-cyano-6-[(1-methylethyl)oxy]-3-pyridinyl}-1,2,4-oxadiazol-3-yl)-1H-indazol-1-yl]butanoic acid
5-(5-{5-chloro-6-[(1-methylethyl)oxy]-3-pyridinyl}-1,2,4-oxadiazol-3-yl)-6-methyl-1H-indazole
5-(5-{5-chloro-6-[(1-methylethyl)oxy]-3-pyridinyl}-1,2,4-oxadiazol-3-yl)-6-fluoro-1H-indazole
4-[5-(5-{5-chloro-6-[(1-methylethyl)oxy]-3-pyridinyl}-1,2,4-oxadiazol-3-yl)-6-fluoro-1H-indazol-1-yl]butanoic acid
4-[5-(5-{5-chloro-6-[(1-methylethyl)oxy]-3-pyridinyl}-1,2,4-oxadiazol-3-yl)-6-fluoro-2H-indazol-2-yl]butanoic acid
6-chloro-5-(5-{5-chloro-6-[(1-methylethyl)oxy]-3-pyridinyl}-1,2,4-oxadiazol-3-yl)-1H-indazole
4-[6-chloro-5-(5-{5-chloro-6-[(1-methylethyl)oxy]-3-pyridinyl}-1,2,4-oxadiazol-3-yl)-1H-indazol-1-yl]butanoic acid
4-[6-chloro-5-(5-{5-chloro-6-[(1-methylethyl)oxy]-3-pyridinyl}-1,2,4-oxadiazol-3-yl)-2H-indazol-2-yl]butanoic acid
5-(5-{5-chloro-6-[(1-methylethyl)oxy]-3-pyridinyl}-1,2,4-oxadiazol-3-yl)-6-ethyl-1H-indazole
4-[5-(5-{5-chloro-6-[(1-methylethyl)oxy]-3-pyridinyl}-1,2,4-oxadiazol-3-yl)-6-ethyl-2H-indazol-2-yl]butanoic acid
4-[5-(5-{5-chloro-6-[(1-methylethyl)oxy]-3-pyridinyl}-1,2,4-oxadiazol-3-yl)-6-ethyl-1H-indazol-1-yl]butanoic acid
5-(5-{5-chloro-6-[(1-methylethyl)oxy]-3-pyridinyl}-1,2,4-oxadiazol-3-yl)-6-(methyloxy)-1H-indazole
3-[5-(5-{5-cyano-6-[(1-methylethyl)oxy]-3-pyridinyl}-1,2,4-oxadiazol-3-yl)-1H-indazol-1-yl]propanoic acid
4-[6-chloro-5-(5-{5-cyano-6-[(1-methylethyl)oxy]-3-pyridinyl}-1,2,4-oxadiazol-3-yl)-1H-indazol-1-yl]butanoic acid
4-[6-chloro-5-(5-{3-cyano-4-[(1-methylethyl)oxy]phenyl}-1,2,4-oxadiazol-3-yl)-1H-indazol-1-yl]butanoic acid
4-[5-(5-{3-cyano-4-[(1-methylethyl)oxy]phenyl}-1,2,4-oxadiazol-3-yl)-6-methyl-1H-indazol-1-yl]butanoic acid
4-[5-(5-{3-chloro-4-[(1-methylethyl)oxy]phenyl}-1,2,4-oxadiazol-3-yl)-6-methyl-1H-indazol-1-yl]butanoic acid
4-[5-(5-{3-cyano-4-[(1-methylethyl)oxy]phenyl}-1,2,4-oxadiazol-3-yl)-6-ethyl-1H-indazol-1-yl]butanoic acid
4-[5-(5-{3-cyano-4-[(1-methylethyl)oxy]phenyl}-1,2,4-oxadiazol-3-yl)-6-(methyloxy)-1H-indazol-1-yl]butanoic acid
and salts thereof.

3. A method of treating a mammal having a condition or disorder mediated by a S1P1 receptor comprising administering a therapeutically effective amount of a compound according to claim 1 to said mammal for the treatment of said condition or disorder.

4. A method according to claim 3, wherein the condition or disorder is multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

5. A method according to claim 4, wherein the condition is multiple sclerosis.

6-8. (canceled)

9. A pharmaceutical composition comprising a compound of formula (I) or a pharmaceutically acceptable salt thereof according to claim 1.

10-11. (canceled)

12. A method of treating a mammal having a condition or disorder mediated by a S1P1 receptor comprising administering a therapeutically effective amount of a compound according to claim 2 to said mammal for the treatment of said condition or disorder.

13. A method according to claim 12, wherein the condition or disorder is multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

14. A method according to claim 13, wherein the condition is multiple sclerosis.

15. A pharmaceutical composition comprising a compound of formula (I) or a pharmaceutically acceptable salt thereof according to claim 2.
